(19) United States
(12) Patent Application Publication
(10) Pub. No.: US 2024/0318237 A1
(43) Pub. Date: Sep. 26, 2024

(54) RNASE H2 MUTANTS THAT REDUCE PRIMER DIMERS AND OFF-TARGET AMPLIFICATION IN RHPCR-BASED AMPLICON SEQUENCING WITH HIGH-FIDELITY DNA POLYMERASES

(71) Applicant: Integrated DNA Technologies, Inc., Coralville, IA (US)

(72) Inventors: Joseph Dobosy, Coralville, IA (US); John Froehlig, JR., North Liberty, IA (US); Katherine Perschbacher, Coralville, IA (US); Kristin Beltz, Cedar Rapids, IA (US); Scott Rose, Coralville, IA (US); Mark Aaron Behlke, Coralville, IA (US)

(73) Assignee: Integrated DNA Technologies, Inc., Coralville, IA (US)

(21) Appl. No.: 18/667,876

(22) Filed: May 17, 2024

Related U.S. Application Data
(63) Continuation of application No. 17/558,994, filed on Dec. 22, 2021, now Pat. No. 12,024,742.

(51) Int. Cl. C12Q 1/6848; C12N 9/22; C12Q 1/6853; C12Q 1/686

(52) U.S. Cl. CPC C12Q 1/6848; C12N 9/22; C12Y 301/26004; C12Q 1/6853; C12Q 1/686

(57) ABSTRACT

The present invention pertains to a kit for producing an extended primer, comprising at least one container providing a hybrid RNase H2 protein, including a hybrid RNase H2 protein comprising fragments of amino acid sequences from Pyrococcus abyssi (P. a.), Thermococcus kodakarensis (T. kod), and Pyrococcus furiosus organisms; a hybrid RNase H2 protein comprises amino acid residues 26-40 and residues 100-120 of T. kod RNase H2; a hybrid RNase H2 protein is selected from SEQ ID NO: 2 and 3; and a hybrid RNase H2 protein is selected from SEQ ID NO: 14-20.

### CROSS-REFERENCE TO RELATED APPLICATIONS

This application is a continuation of U.S. patent application Ser. No. 17/558,994, filed Dec. 22, 2021, issued as U.S. Pat. No. 12,024,742 on Jul. 2, 2024, and entitled “RNASE H2 MUTANTS THAT ENHANCE MISMATCH DISCRIMINATION AND ACTIVITY IN HIGH-FIDELITY POLYMERASE BUFFER,” which claims benefit of priority under 35 U.S.C. 119 to U.S. Provisional Patent Application Ser. No. 63/130,548, filed Dec. 24, 2020 and entitled “RNASE H2 MUTANTS THAT ENHANCE MISMATCH DISCRIMINATION AND ACTIVITY IN HIGH-FIDELITY POLYMERASE BUFFER,” and U.S. Provisional Patent Application Ser. No. 63/277,273, filed Nov. 9, 2021 and entitled “RNASE H2 MUTANTS THAT REDUCE PRIMER DIMERS AND OFF-TARGET AMPLIFICATION IN RHPCR-BASED AMPLICON SEQUENCING WITH HIGH-FIDELITY DNA POLYMERASES,” the contents of which are herein incorporated by reference in their entirety.

### FIELD OF THE INVENTION

This invention pertains to Type II RNase H (hereinafter RNase H2) hybrid enzyme variants and methods of cleaving a nucleic acid strand to initiate, assist, monitor or perform biological assays.

### SEQUENCE LISTING

The instant application contains a Sequence Listing that has been submitted in XML format via Patent Center and is hereby incorporated by reference in its entirety. The XML copy, created on Jun. 13, 2024, is named IDT01-018-US-CON.XML, and is 175,513 bytes in size.

### BACKGROUND OF THE INVENTION

The family of RNase H2 enzymes has been extensively characterized. These enzymes have substrate specificity for cleaving a single ribonucleotide embedded within a DNA sequence (in duplex form) (Eder, et al., (1993) Biochimie, 75, 123-126). Interestingly, cleavage occurs on the 5′ side of the RNA residue (See Scheme I). A summary of these enzymes, their properties and applications in biological assays is summarized in U.S. Pat. No. 8,911,948 B2 to Walder et al.

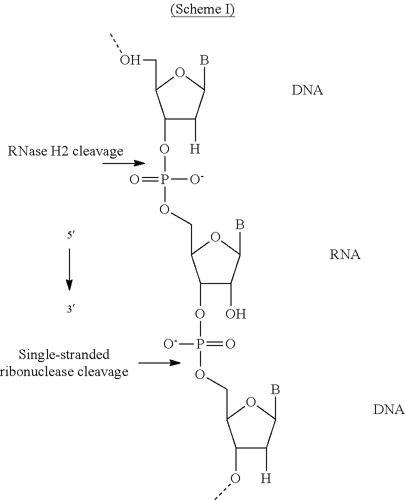

The RNase H2 enzyme isolated from the hyperthermophile Pyrococcus abyssi (P. a.) cleaves on the 5′ side of a ribonucleotide embedded in a nucleic acid strand that is otherwise entirely made of DNA. RNase H2-dependent PCR (rhPCR) improves the specificity of PCR using a thermostable RNase H2 enzyme to cleave a primer containing a single ribonucleotide near the 3′ end, removing a blocking group on the 3′ terminus (Dobosy et al., 2011; U.S. Pat. No. 8,911,948 B2). This primer is initially incapable of extension by DNA polymerase, but upon removal of the blocking group from the 3′ end by RNase H2, the primer is capable of being extended. P. a. RNase H2 is sensitive to single-base mismatches in the DNA-RNA heteroduplex near the ribonucleotide and cleaves a mismatch-containing template at a much-reduced rate, which allows the perfectly matched duplex to be preferentially cleaved and extended. This results in increased specificity in the rhPCR reaction, and lowers primer-dimer formation and other off-target amplifications.

Despite the enhancement rhPCR brings to the specificity of PCR, it currently has limitations. The apparent mismatch discrimination of rhPCR is lower than what should be theoretically achievable. WT P. a. RNase H2 recognizes single-base mismatches directly opposite of the RNA base to a high degree, though with varying efficiency depending on the nature of the mismatch. In addition, the native enzyme has comparatively limited mismatch discrimination at positions directly 5′ or 3′ of the RNA base. Despite this limitation, placing mutations at these locations can be advantageous. As an example, a mismatch directly 5′ of the RNA can be used as a secondary selection step with the use of a discriminatory DNA polymerase (such as H784Q Thermus aquaticus DNA polymerase) in PCR amplification after primer cleavage (See U.S. patent application Ser. No. 15/361,280). Placing a mismatch 3′ of the RNA reduces the likelihood of template conversion, as the mismatch recognition will happen with every cycle rather than once during template conversion.

Despite the utility of rhPCR, mismatch discrimination of wild type P. a. RNase H2 can be “leaky”, resulting in some amplification of primer dimers. Primer dimers generated during amplification of sequencing libraries are problematic because they can bind to the Illumina flow cell and undergo sequencing, but do not provide any meaningful data. High levels of primer dimer decrease the fraction of reads mapping to the target of interest, ultimately reducing assay sensitivity or requiring a significant increase in sequencing cost to generate the number of on-target reads necessary for detection of low frequency variants. Low frequency variant detection can also be affected by the introduction of amplification errors during PCR. The error rate can be lowered with the use of a high fidelity DNA polymerase and optimized buffer conditions. However, these buffer conditions reduce the enzymatic activity and mismatch sensitivity of wild type P. a. RNase H2 in rhPCR.

It has previously been shown that the use of RNase H2 mutants generated by partial recombination of amino acid sequences of wild type P. a. RNase H2 with sequences from other related species results in improved RNase H2 enzymatic activity in high fidelity DNA polymerase buffer. Two of these mutants, Q48R SEL29 RNase H2 and Al07V SEL29 RNase H2, were shown to have improved enzymatic activity using a Thermococcus kodakarensis (KOD) DNA polymerase reaction buffer. In addition, both mutants showed enhanced mismatch discrimination opposite the RNA base, as well as mismatches 3′ and 5′ of the RNA base, compared to wild type P. a. RNase H2. See U.S. Provisional Patent Application Ser. No. 63/130,548, filed Dec. 24, 2020, and entitled “RNASE H2 MUTANTS THAT ENHANCE MISMATCH DISCRIMINATION AND ACTIVITY IN HIGH-FIDELITY POLYMERASE BUFFER” (Attorney Docket No. IDT01-018-PRO), the contents of which is incorporated by reference in its entirety.

The present disclosure pertains to one of these novel hybrid RNase H2 enzyme variants, Q48R SEL29 RNase H2, in a multiplex rhAmpSeq workflow containing a high-fidelity DNA polymerase and buffer. Compared to wild type P. a. RNase H2, Q48R SEL29 RNase H2 reduces the primer dimer produced during PCR amplification, thereby improving the mapping rate and on-target rate. While improving these metrics, Q48R SEL29 RNase H2 had no effect on other critical sequencing metrics including amplicon uniformity, amplicon dropout rates, and amplicon uniformity distributions.

### BRIEF SUMMARY OF THE INVENTION

In a first aspect, The present invention pertains to a kit for producing an extended primer, comprising at least one container providing a hybrid RNase H2 protein, including a hybrid RNase H2 protein comprising fragments of amino acid sequences from Pyrococcus abyssi (P. a.), Thermococcus kodakarensis (T. kod), and Pyrococcus furiosus organisms; a hybrid RNase H2 protein comprises amino acid residues 26-40 and residues 100-120 of T. kod RNase H2; a hybrid RNase H2 protein is selected from SEQ ID NO: 2 and 3; and a hybrid RNase H2 protein is selected from SEQ ID NO: 14-20.

### BRIEF DESCRIPTION OF THE DRAWINGS

FIG. 1 depicts an exemplary plot showing that the dimer rate is lowered at all enzyme concentrations using the mutant Q48R SEL29 RNase H2 enzyme compared to the wild type P. a. RNase H2 enzyme (panel A); exemplary data that the mapping rate is higher at all RNase H2 enzyme concentrations with the mutant Q48R SEL29 RNase H2 enzyme compared to the wild type P. a. RNase H2 enzyme (panel B); exemplary data showing that the on target rate is higher at all enzyme concentrations with the mutant Q48R SEL29 RNase H2 enzyme compared to wild type P. a. RNase H2 enzyme (panel C).

FIG. 2 depicts examples of mean normalized dimer count for per each primer pair identified, wherein the mutant Q48R SEL29 RNase H2 enzyme reduces most primer dimers identified by half compared to wild type P. a. RNase H2 enzyme.

FIG. 3 depicts exemplary data Amplicon Uniformity≥0.2× and Amplicon Uniformity≤0.05× (Dropout Rate), wherein the yield of libraries is comparable at all concentrations between the mutant Q48R SEL29 RNase H2 enzyme and the wild type P. a. RNase H2 enzyme (panel A); the overall amplicon uniformity≥0.2× of mutant Q48R SEL29 RNase H2 enzyme being comparable to wild type P. a. RNase H2 enzyme at the various concentrations tested (panel B); and amplicon drop out rates being similar in all titration concentrations tested between the mutant Q48R SEL29 RNase H2 enzyme and wild type P. a. RNase H2 enzyme (panel C).

FIG. 4 depicts exemplary data showing uniformity distribution (percent of amplicons that have coverage in ranges 0-0.1×, 0.1-0.2×, 0.2×-0.5×, 0.5×-1.5×, 1.5×-2.5×, and 2.5-5× compared to the amplicon mean coverage) being comparable between the mutant Q48R SEL29 RNase H2 enzyme and the wild type P. a. RNase H2 enzyme.

### DETAILED DESCRIPTION OF THE INVENTION

The current invention provides novel hybrid RNase H2 enzyme variants that enhance enzymatic activity during rhPCR using certain DNA polymerase buffers while retaining their ability to retain or enhance the mismatch discrimination in a duplex template. The RNase H2 enzyme hybrids combine fragments of amino acid sequences from Pyrococcus abyssi (P. a.), Thermococcus kodakarensis (T. kod), and Pyrococcus furiosus organisms. The resultant hybrid RNase H2 enzymes, as well as select mutants based upon these enzymes, enhance mismatch discrimination significantly. In particular, the Q48R SEL29 RNase H2 (SEQ ID NO.: 18) is shown to produce product mixtures having a reduced population of primer dimer species relative to a product mixture produced with a wild-type P. a. RNase H2 (SEQ ID NO.: 1) in methods of conducting primer extension, performing rhPCR, amplifying a target DNA sequence, performing massively parallel sequencing, detecting a SNP-containing nucleic acid template from an amplicon library of nucleic acid templates, and performing a loop-mediated amplification reaction, among others.

### Definitions

To aid in understanding the invention, several terms are defined below.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double-and single-stranded DNA, as well as double-and single-stranded RNA. For use in the present invention, an oligonucleotide also can comprise nucleotide analogs in which the base, sugar or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1 (3): 165-187, incorporated herein by reference.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (e.g., a DNA polymerase or reverse transcriptase) in an appropriate buffer and at a suitable temperature. Primer extension can also be carried out in the absence of one or more of the nucleotide triphosphates in which case an extension product of limited length is produced. As used herein, the term “primer” is intended to encompass the oligonucleotides used in ligation-mediated reactions, in which one oligonucleotide is “extended” by ligation to a second oligonucleotide that hybridizes at an adjacent position. Thus, the term “primer extension”, as used herein, refers to both the polymerization of individual nucleoside triphosphates using the primer as a point of initiation of DNA synthesis and to the ligation of two oligonucleotides to form an extended product.

A primer is preferably a single-stranded DNA. The appropriate length of a primer depends on the intended use of the primer but typically ranges from 6 to 50 nucleotides, preferably from 15-35 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product. The region of the primer that is sufficiently complementary to the template to hybridize is referred to herein as the hybridizing region.

The terms “target, “target sequence”, “target region”, and “target nucleic acid,” as used herein, are synonymous and refer to a region or sequence of a nucleic acid which is to be amplified, sequenced or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26 (3/4):227-259; and Owczarzy et al., 2008, Biochemistry, 47:5336-5353, which are incorporated herein by reference).

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two step cycles have a high temperature denaturation step followed by a hybridization/elongation (or ligation) step. Three step cycles comprise a denaturation step followed by a hybridization step followed by a separate elongation or ligation step.

As used herein, a “polymerase” refers to an enzyme that catalyzes the polymerization of nucleotides. Generally, the enzyme will initiate synthesis at the 3′-end of the primer annealed to a nucleic acid template sequence. “DNA polymerase” catalyzes the polymerization of deoxyribonucleotides. Known DNA polymerases include, for example, Pyrococcus furiosus (Pfu) DNA polymerase (Lundberg et al., 1991, Gene, 108:1), E. coli DNA polymerase I (Lecomte and Doubleday, 1983, Nucleic Acids Res. 11:7505), T7 DNA polymerase (Nordstrom et al., 1981, J. Biol. Chem. 256:3112), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent DNA polymerase, Cariello et al., 1991, Nucleic Acids Res, 19:4193), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127:1550), Thermoccus Pyrococcus kodakaraensis KOD DNA polymerase (Takagi et al., 1997, Appl. Environ. Microbiol. 63:4504), JDF-3 DNA polymerase (Patent application WO 0132887), and Pyrococcus GB-D (PGB-D) DNA polymerase (Juncosa-Ginesta et al., 1994, Biotechniques, 16:820). The polymerase activity of any of the above enzymes can be determined by means well known in the art.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

The term “non-specific amplification,” as used herein, refers to the amplification of nucleic acid sequences other than the target sequence that results from primers hybridizing to sequences other than the target sequence and then serving as a substrate for primer extension. The hybridization of a primer to a non-target sequence is referred to as “non-specific hybridization” and is apt to occur especially during the lower temperature, reduced stringency, pre-amplification conditions, or in situations where there is a variant allele in the sample having a very closely related sequence to the true target as in the case of a single nucleotide polymorphism (SNP).

The term “3′-mismatch discrimination” refers to a property of a DNA polymerase to distinguish a fully complementary sequence from a mismatch-containing (nearly complementary) sequence where the nucleic acid to be extended (for example, a primer or other oligonucleotide) has a mismatch at the 3′ end of the nucleic acid compared to the template to which the nucleic acid hybridizes. In some embodiments, the nucleic acid to be extended comprises a mismatch at the 3′ end relative to the fully complementary sequence.

The term “3′-mismatch discrimination assay” refers to an assay to discern the present of improved specificity in amplification of a target DNA sequence when the target DNA sequence is interrogated with two primers having substantially identical sequence except for the occurrence of one of more nucleotide residue having different base composition at or near their respective 3′-ends. For example, a first primer having 3′-end sequences with perfect complementarity to the target DNA sequence is considered a 3′-matched primer and a second primer having a 3′-end sequences having at least one nucleotide base non-complementarity to the target DNA sequence is considered a 3′-mismatched primer. An example of a 3′-mismatch discrimination assay is provided in many of the examples, such as EXAMPLE 4 Tables 10 and 11, among others.

The term “primer dimer,” as used herein, refers to a template-independent non-specific amplification product, which is believed to result from primer extensions wherein another primer serves as a template. Although primer dimers frequently appear to be a concatamer of two primers, i.e., a dimer, concatamers of more than two primers also occur. The term “primer dimer” is used herein generically to encompass a template-independent non-specific amplification product.

The term “reaction mixture,” as used herein, refers to a solution containing reagents necessary to carry out a given reaction. An “amplification reaction mixture”, which refers to a solution containing reagents necessary to carry out an amplification reaction, typically contains oligonucleotide primers and a DNA polymerase or ligase in a suitable buffer. A “PCR reaction mixture” typically contains oligonucleotide primers, a DNA polymerase (most typically a thermostable DNA polymerase), dNTP's, and a divalent metal cation in a suitable buffer. A reaction mixture is referred to as complete if it contains all reagents necessary to enable the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction components that includes the blocked primers of the invention.

For the purposes of this invention, the terms “non-activated” or “inactivated,” as used herein, refer to a primer or other oligonucleotide that is incapable of participating in a primer extension reaction or a ligation reaction because either DNA polymerase or DNA ligase cannot interact with the oligonucleotide for their intended purposes. In some embodiments when the oligonucleotide is a primer, the non-activated state occurs because the primer is blocked at or near the 3′-end so as to prevent primer extension. When specific groups are bound at or near the 3′-end of the primer, DNA polymerase cannot bind to the primer and extension cannot occur. A non-activated primer is, however, capable of hybridizing to a substantially complementary nucleotide sequence.

For the purposes of this invention, the term “activated,” as used herein, refers to a primer or other oligonucleotide that is capable of participating in a reaction with DNA polymerase or DNA ligase. A primer or other oligonucleotide becomes activated after it hybridizes to a substantially complementary nucleic acid sequence and is cleaved to generate a functional 3′- or 5′-end so that it can interact with a DNA polymerase or a DNA ligase. For example, when the oligonucleotide is a primer, and the primer is hybridized to a template, a 3′-blocking group can be removed from the primer by, for example, a cleaving enzyme such that DNA polymerase can bind to the 3′ end of the primer and promote primer extension.

The term “cleavage domain” or “cleaving domain,” as used herein, are synonymous and refer to a region located between the 5′ and 3′ end of a primer or other oligonucleotide that is recognized by a cleavage compound, for example a cleavage enzyme, that will cleave the primer or other oligonucleotide. For the purposes of this invention, the cleavage domain is designed such that the primer or other oligonucleotide is cleaved only when it is hybridized to a complementary nucleic acid sequence, but will not be cleaved when it is single-stranded. The cleavage domain or sequences flanking it may include a moiety that a) prevents or inhibits the extension or ligation of a primer or other oligonucleotide by a polymerase or a ligase, b) enhances discrimination to detect variant alleles, or c) suppresses undesired cleavage reactions. One or more such moieties may be included in the cleavage domain or the sequences flanking it.

The term “RNase H cleavage domain,” as used herein, is a type of cleavage domain that contains one or more ribonucleic acid residue or an alternative analog that provides a substrate for an RNase H. An RNase H cleavage domain can be located anywhere within a primer or oligonucleotide, and is preferably located at or near the 3′-end or the 5′-end of the molecule.

An “RNase H2 cleavage domain” may contain one RNA residue, a sequence of contiguously linked RNA residues or RNA residues separated by DNA residues or other chemical groups. In one embodiment, the RNase H2 cleavage domain is a 2′-fluoronucleoside residue. In a more preferred embodiment, the RNase H2 cleavable domain includes two adjacent 2′-fluoro residues.

The term “blocked primer,” as used herein, refers to a primer which possesses, at minimum, a cleaving domain suitable for hybridizing sufficiently to a target sequence, a cleavable domain, and a blocking group preventing extension from the 3′ end of the primer until cleavage occurs. In the preferred embodiment, the cleavable domain is an RNase H cleavage domain, and the blocking group is a propanediol (C3) spacer.

The terms “cleavage compound,” or “cleaving agent” as used herein, refers to any compound that can recognize a cleavage domain within a primer or other oligonucleotide, and selectively cleave the oligonucleotide based on the presence of the cleavage domain. The cleavage compounds utilized in the invention selectively cleave the primer or other oligonucleotide comprising the cleavage domain only when it is hybridized to a substantially complementary nucleic acid sequence, but will not cleave the primer or other oligonucleotide when it is single stranded. The cleavage compound cleaves the primer or other oligonucleotide within or adjacent to the cleavage domain. The term “adjacent,” as used herein, means that the cleavage compound cleaves the primer or other oligonucleotide at either the 5′-end or the 3′ end of the cleavage domain. Cleavage reactions preferred in the invention yield a 5′-phosphate group and a 3′-OH group.

In a preferred embodiment, the cleavage compound is a “cleaving enzyme.” A cleaving enzyme is a protein or a ribozyme that is capable of recognizing the cleaving domain when a primer or other nucleotide is hybridized to a substantially complementary nucleic acid sequence, but that will not cleave the complementary nucleic acid sequence (i.e., it provides a single strand break in the duplex). The cleaving enzyme will also not cleave the primer or other oligonucleotide comprising the cleavage domain when it is single stranded. Examples of cleaving enzymes are RNase H enzymes and other nicking enzymes.

The term “nicking,” as used herein, refers to the cleavage of only one strand of the double-stranded portion of a fully or partially double-stranded nucleic acid. The position where the nucleic acid is nicked is referred to as the “nicking site” (NS). A “nicking agent” (NA) is an agent that nicks a partially or fully double-stranded nucleic acid. It may be an enzyme or any other chemical compound or composition. In certain embodiments, a nicking agent may recognize a particular nucleotide sequence of a fully or partially double-stranded nucleic acid and cleave only one strand of the fully or partially double-stranded nucleic acid at a specific position (i.e., the NS) relative to the location of the recognition sequence. Such nicking agents (referred to as “sequence specific nicking agents”) include, but are not limited to, nicking endonucleases (e.g., N.BstNB).

A “nicking endonuclease” (NE), as used herein, thus refers to an endonuclease that recognizes a nucleotide sequence of a completely or partially double-stranded nucleic acid molecule and cleaves only one strand of the nucleic acid molecule at a specific location relative to the recognition sequence. In such a case the entire sequence from the recognition site to the point of cleavage constitutes the “cleavage domain”.

The term “blocking group,” as used herein, refers to a chemical moiety that is bound to the primer or other oligonucleotide such that an amplification reaction does not occur. For example, primer extension and/or DNA ligation does not occur. Once the blocking group is removed from the primer or other oligonucleotide, the oligonucleotide is capable of participating in the assay for which it was designed (PCR, ligation, sequencing, etc.). Thus, the “blocking group” can be any chemical moiety that inhibits recognition by a polymerase or DNA ligase. The blocking group may be incorporated into the cleavage domain but is generally located on either the 5′- or 3′-side of the cleavage domain. The blocking group can be comprised of more than one chemical moiety. In the present invention the “blocking group” is typically removed after hybridization of the oligonucleotide to its target sequence.

The term “fluorescent generation probe” refers either to a) an oligonucleotide having an attached fluorophore and quencher, and optionally a minor groove binder or to b) a DNA binding reagent such as SYB® Green dye.

The terms “fluorescent label” or “fluorophore” refers to compounds with a fluorescent emission maximum between about 350 and 900 nm. A wide variety of fluorophores can be used, including but not limited to: 5-FAM (also called 5-carboxyfluorescein; also called Spiro (isobenzofuran-1 (3H), 9′-(9H) xanthene)-5-carboxylic acid,3′,6′-dihydroxy-3-oxo-6-carboxyfluorescein); 5-Hexachloro-Fluorescein; ([4,7,2′,4′,5′,7′-hexachloro-(3′,6′-dipivaloyl-fluoresceinyl)-6-carboxylic acid]); 6-Hexachloro-Fluorescein; ([4,7,2′,4′,5′,7′-hexachloro-(3′,6′-dipivaloylfluoresceinyl)-5-carboxylic acid]); 5-Tetrachloro-Fluorescein; ([4,7,2′,7′-tetra-chloro-(3′,6′-dipivaloylfluoresceinyl)-5-carboxylic acid]); 6-Tetrachloro-Fluorescein; ([4,7,2′,7′-tetrachloro-(3′,6′-dipivaloylfluoresceinyl)-6-carboxylic acid]); 5-TAMRA (5-carboxytetramethylrhodamine); Xanthylium, 9-(2,4-dicarboxyphenyl)-3,6-bis (dimethyl-amino); 6-TAMRA (6-carboxytetramethylrhodamine); 9-(2,5-dicarboxyphenyl)-3,6-bis (dimethylamino); EDANS (5-((2-aminoethyl) amino) naphthalene-1-sulfonic acid); 1,5-IAEDANS (5-((((2-iodoacetyl) amino) ethyl) amino) naphthalene-1-sulfonic acid); Cy5 (Indodicarbocyanine-5); Cy3 (Indo-dicarbocyanine-3); and BODIPY FL (2,6-dibromo-4,4-difluoro-5,7-dimethyl-4-bora-3a,4a-diaza-s-indacene-3-proprionic acid); Quasar®-670 dye (Biosearch Technologies); Cal Fluor® Orange dye (Biosearch Technologies); Rox dyes; Max dyes (Integrated DNA Technologies), as well as suitable derivatives thereof.

As used herein, the term “quencher” refers to a molecule or part of a compound, which is capable of reducing the emission from a fluorescent donor when attached to or in proximity to the donor. Quenching may occur by any of several mechanisms including fluorescence resonance energy transfer, photo-induced electron transfer, paramagnetic enhancement of intersystem crossing, Dexter exchange coupling, and exciton coupling such as the formation of dark complexes. Fluorescence is “quenched” when the fluorescence emitted by the fluorophore is reduced as compared with the fluorescence in the absence of the quencher by at least 10%, for example, 15%, 20%, 30%, 40%, 50%, 60%, 70%, 80%, 90%, 95%, 98%, 99%, 99.9% or more. A number of commercially available quenchers are known in the art, and include but are not limited to DABCYL, Black Hole™ Quenchers (BHQ-1, BHQ-2, and BHQ-3), Iowa Black® FQ and Iowa Black® RQ. These are so-called dark quenchers. They have no native fluorescence, virtually eliminating background problems seen with other quenchers such as TAMRA that is intrinsically fluorescent.

The term “ligation” as used herein refers to the covalent joining of two polynucleotide ends. In various embodiments, ligation involves the covalent joining of a 3′ end of a first polynucleotide (the acceptor) to a 5′ end of a second polynucleotide (the donor). Ligation results in a phosphodiester bond being formed between the polynucleotide ends. In various embodiments, ligation may be mediated by any enzyme, chemical, or process that results in a covalent joining of the polynucleotide ends. In certain embodiments, ligation is mediated by a ligase enzyme.

As used herein, “ligase” refers to an enzyme that is capable of covalently linking the 3′ hydroxyl group of one polynucleotide to the 5′ phosphate group of a second polynucleotide. Examples of ligases include E. coli DNA ligase, T4 DNA ligase, etc.

The ligation reaction can be employed in DNA amplification methods such as the “ligase chain reaction” (LCR), also referred to as the “ligase amplification reaction” (LAR), see Barany, Proc. Natl. Acad. Sci., 88:189 (1991); and Wu and Wallace, Genomics 4:560 (1989) incorporated herein by reference. In LCR, four oligonucleotides, two adjacent oligonucleotides which uniquely hybridize to one strand of the target DNA, and a complementary set of adjacent oligonucleotides, that hybridize to the opposite strand are mixed and DNA ligase is added to the mixture. In the presence of the target sequence, DNA ligase will covalently link each set of hybridized molecules. Importantly, in LCR, two oligonucleotides are ligated together only when they base-pair with sequences without gaps. Repeated cycles of denaturation, hybridization and ligation amplify a short segment of DNA. A mismatch at the junction between adjacent oligonucleotides inhibits ligation. As in other oligonucleotide ligation assays this property allows LCR to be used to distinguish between variant alleles such as SNPs. LCR has also been used in combination with PCR to achieve enhanced detection of single-base changes, see Segev, PCT Public. No. WO9001069 (1990).

The term “codon-optimized” as that term modifies a particular nucleic acid encoding a polypeptide refers to inclusion of preferred codons for efficient expression in a given host cell, such as a given microorganism (e.g., E. coli, S. cerevisae, among others) or a mammalian cells (e.g., human cells, such as HeLa, COS cells, among others). Such preferred codons are well known in the art based upon codon bias tables developed for a variety of organisms. The polynucleotides that encode polypeptides of the present invention include those having codon-optimized open reading frames for any known organism in which codon bias tables have been developed or for which such tables can be readily discerned from empirical determination.

The phrase “BaseX PCR amplification method” refers to a highly efficient nucleic acid amplification that allows for a greater than 2-fold increase of amplification product for each amplification cycle and therefore increased sensitivity and speed over conventional PCR. This method is disclosed in U.S. Pat. No. 10,273,534 (B2), entitled “Exponential base-greater-than-2 nucleic acid amplification” to R. Higuchi (Applicant: Cepheid), which issued Apr. 30, 2019, the contents of which is herein incorporated by reference in its entirety.

The phrases “fusion protein” or “fusion polypeptide” refers to the inclusion of extra amino acid information that is not native to the protein to which the extra amino acid information is covalently attached. Such extra amino acid information may include tags that enable purification or identification of the fusion protein. Such extra amino acid information may include peptides that enable the fusion proteins to be transported into cells and/or transported to specific locations within cells. Examples of tags for these purposes include the following: AviTag, which is a peptide allowing biotinylation by the enzyme BirA so the protein can be isolated by streptavidin; Calmodulin-tag, which is a peptide bound by the protein calmodulin; polyglutamate tag, which is a peptide binding efficiently to anion-exchange resin such as Mono-Q; E-tag, which is a peptide recognized by an antibody; FLAG-tag, which is a peptide recognized by an antibody; HA-tag, which is a peptide from hemagglutinin recognized by an antibody; His-tag, which is typically 5-10 histidines bound by a nickel or cobalt chelate; Myc-tag, which is a peptide derived from c-myc recognized by an antibody; NE-tag, which is a novel 18-amino-acid synthetic peptide recognized by a monoclonal IgG1 antibody, which is useful in a wide spectrum of applications including Western blotting, ELISA, flow cytometry, immunocytochemistry, immunoprecipitation, and affinity purification of recombinant proteins; S-tag, which is a peptide derived from Ribonuclease A; SBP-tag, which is a peptide which binds to streptavidin; Softag 1, which is intended for mammalian expression; Softag 3, which is intended for prokaryotic expression; Strep-tag, which is a peptide which binds to streptavidin or the modified streptavidin called streptactin (Strep-tag II); TC tag, which is a tetracysteine tag that is recognized by FlAsH and ReAsH biarsenical compounds; V5 tag, which is a peptide recognized by an antibody; VSV-tag, a peptide recognized by an antibody; Xpress tag; Isopeptag, which is a peptide which binds covalently to pilin-C protein; SpyTag, which is a peptide which binds covalently to SpyCatcher protein; SnoopTag, a peptide which binds covalently to SnoopCatcher protein; BCCP (Biotin Carboxyl Carrier Protein), which is a protein domain biotinylated by BirA to enable recognition by streptavidin; Glutathione-S-transferase-tag, which is a protein that binds to immobilized glutathione; Green fluorescent protein-tag, which is a protein which is spontaneously fluorescent and can be bound by antibodies; HaloTag, which is a mutated bacterial haloalkane dehalogenase that covalently attaches to a reactive haloalkane substrate to allow attachment to a wide variety of substrates; Maltose binding protein-tag, a protein which binds to amylose agarose; Nus-tag; Thioredoxin-tag; and Fc-tag, derived from immunoglobulin Fc domain, which allows dimerization and solubilization and can be used for purification on Protein-A Sepharose. Nuclear localization signals (NLS), such as those obtained from SV40, allow for proteins to be transported to the nucleus immediately upon entering the cell. Given that the native Cas9 protein is bacterial in origin and therefore does not naturally comprise a NLS motif, addition of one or more NLS motifs to the recombinant Cas9 protein is expected to show improved genome editing activity when used in eukaryotic cells where the target genomic DNA substrate resides in the nucleus. One skilled in the art would appreciate these various fusion tag technologies, the particular amino acid sequences involved, as well as how to make and use fusion proteins that include them. In one embodiment, highly preferred fusion proteins or fusion polypeptides include a His-tag motif, though one skilled in the art will appreciate that other tags can be included, as noted above. The present invention includes fusion proteins or fusion polypeptides as well as versions of the original mutated versions of the corresponding protein or polypeptide lacking additional amino acid sequence information.

### Novel Hybrid RNase H2 Enzyme Variants Generated by Recombination Reshuffling of Amino Acid Sequences From Known RNase H2 Enzymes

Two hybrid RNase H2 enzymes with novel and useful properties were generated via partial recombination (“shuffling”) of amino acid sequences from three RNase H2 enzymes, including Pyrococcus abyssi, Thermococcus kodakarensis, and Pyrococcus furiosus. and can result in hybrid RNase H2 enzymes. In particular, two mutant RNase H2 enzymes, SEL28 RNase H2 (SEQ ID NO.: 89) and SEL29 RNase H2 (SEQ ID NO.: 90) that combine fragments of amino acid sequences from Pyrococcus abyssi, Thermococcus kodakarensis, and Pyrococcus furiosus organisms have been discovered to enhance mismatch discrimination significantly. The mutants were selected from a library created by random shuffling of RNase H2 sequences. Both mutant enzymes contain fragments of amino acid residues 26-40 and residues 100-120 of T. kod RNase H2, although other changes are also present. Based on structural homologies to known crystal structures (Muroya et al., 2001; Rychlik et al., 2010), these residues likely make contacts with the bound DNA duplex. Without being bound to any particular theory, it can be hypothesized that residues 26-40 and residues 100-120 of T. kod RNase H2 will change the enzyme binding pocket for substrate duplex resulting in changes of the binding affinity and catalysis of nucleic acid cleavage. The amino acid sequences of the wild-type P. ab. RNase H2 protein (SEQ ID NO.: 88), hybrid SEL28 RNase H2 protein (SEQ ID NO.: 89) and hybrid SEL29 RNase H2 protein (SEQ ID NO.: 90) are depicted in Table 1. The corresponding (His) 6-tagged amino acid sequences of the wild-type P. ab. RNase H2 protein (SEQ ID NO.: 1), hybrid SEL28 RNase H2 protein (SEQ ID NO.: 2) and hybrid SEL29 RNase H2 protein (SEQ ID NO.: 3) were prepared as well and served as the basis for generating additional mutant RNase H2 proteins (see Table 3).

TABLE 1
Amino acid sequences for the
hybrid RNase H2 proteins.

| SEQ ID | Protein | |
|---|---|---|
| NO.: | Description | Sequence¹ |
| 88 | WT P. ab. | MKVAGADEAGRGPVI |
| | RNase H2 | GPLVIVAAVVEEDKI |
| | | RSLTKLGVKDSKQLT |
| | | PAQREKLFDEIVKVL |
| | | DDYSVVIVSPQDIDG |
| | | RKGSMNELEVENFVK |
| | | ALNSLKVKPEVIYID |
| | | SADVKAERFAENIRS |
| | | RLAYEAKVVAEHKAD |
| | | AKYEIVSAASILAKV |
| | | IRDREIEKLKAEYGD |
| | | FGSGYPSDPRTKKWL |
| | | EEWYSKHGNFPPIVR |
| | | RTWDTAKKIEEKFKR |
| | | AQLTLDNFLKRFRN |
| 89 | SEL28 | MKVAGADEAGRGPVI |
| | RNase H2 | GPLVIVAAVVDENSL |
| | | PKLEELKVRDSKKLT |
| | | PKRREKLFDEIVKVL |
| | | DDYSVVIVSPQDIDG |
| | | RKGSMNELEVENFVK |
| | | ALNSLKVKPDVIYAD |
| | | AADVDEERFARELGE |
| | | RLNFEAEVVAKHKAD |
| | | DIFPVVSAASILAKV |
| | | IRDREIEKLKAEYGD |
| | | FGSGYPSDPRTKKWL |
| | | EEWYSKHGNFPPIVR |
| | | RTWDTAKKIEEKFKR |
| | | AQLTLDKFFKKP |
| 90 | SEL29 | MKVAGIDEAGRGPAI |
| | RNase H2 | GPLVIVAAVVDENSL |
| | | PKLEELKVRDSKKLT |
| | | PAQREKLFDEIVKVL |
| | | DDYSVVIVSPQDIDG |
| | | RKGSMNELEVENFVK |
| | | ALNSLKVKPDVIYAD |
| | | AADVDEERFARELGE |
| | | RLNFEAKVVAEHKAD |
| | | AKYEIVSAASILAKV |
| | | IRDREIEKLKAEYGD |
| | | FGSGYPSDPRTRAFL |
| | | ENYYREHGEFPPIVR |
| | | KGWKTLKKIAEKVES |
| | | EKKAEERQATLDRYF |
| | | RKV |

¹Amino acids identical to wild-type Pyrococcus abyssi RNase H2 are shown as non-underlined, non-bold sequence. Amino acids that appear to originate from Thermococcus kodakarensis and Pyrococcus furiosus RNase H2 sequences are shown in bold and underlined respectively.

The resultant hybrid SEL28 and SEL29 RNase H2 enzymes encoded by SEQ ID NOs.: 89 and 90 improve mismatch discrimination when the mismatch is located at RNA nucleotide, but to various degrees and with different specificities. (Data not shown). Likewise, the SEL28 and SEL29 mutant RNase H2 enzymes encoded by SEQ ID NOs.: 89 and 90 improve mismatch discrimination 5′ of the RNA nucleotide. (Data not shown.)

rhPCR could also be performed using a high-fidelity DNA polymerase—such as the DNA polymerase from Pyrococcus furiosus and Thermococcus kodakarensis (KOD)—instead of the DNA polymerase from Thermus aquaticus (Taq). However, WT P. a. RNase H2, SEL28 RNase H2, and SEL29 RNase H2 have limited activity in rhPCR using high-fidelity polymerase and the associated reaction buffer. The optimal reaction buffer for Taq DNA polymerase differs substantially from the optimal buffer for KOD DNA polymerase. Mutations in RNase H2 could be found that have greater tolerance for components in the reaction buffer for KOD DNA polymerase. We show that Q48R, A107V, and P13S/A107V when added to hybrid mutant SEL28 or SEL29 RNase H2 enhances enzymatic activity using KOD DNA polymerase reaction buffer.

The present invention relates to mutant RNase H2 enzymes that enhance enzymatic activity during rhPCR using KOD DNA polymerase and its optimal reaction buffer. Mutations in the 48^{th} and 107^{th} amino acid of SEL29 RNase H2 have been shown to have this improved activity. A screen of seven point mutants with a background of either SEL28 RNase H2 or SEL29 RNase H2 were performed, and Q48R SEL29 RNase H2 and A107V SEL29 RNase H2 were shown to enhance enzymatic activity using KOD DNA polymerase reaction buffer. These also were shown to retain or enhance the mismatch discrimination capabilities of the SEL29 RNase H2.

### RNase H2-Mediated POR

The hybrid RNase H2 mutant proteins disclosed herein can be used in a variety of PCR applications. RNase H2-dependent PCR is a method for increasing PCR specificity and eliminating primer-dimers by using RNase H2 from Pyrococcus abyssi or related organisms and DNA primers that contain a single ribonucleotide residue and a 3′ blocking moiety (“blocked-cleavable primers”). The blocked-cleavable primers are activated when cleaved by the RNase H2 enzyme. Cleavage occurs on the 5′ side of the RNA base after primer hybridization to the target DNA. Because the primers can only be cleaved after they hybridize to the perfectly matched target sequence, primer-dimers are reduced. The requirement for high target complementarity reduces amplification of closely related sequences.

In this regard, the hybrid RNase H2 mutant proteins are particularly amendable to enhancing performance in generating high quality genomic amplicon libraries for high throughput multiplex sequencing applications, such as next-generation sequencing applications (NGS). In particular, Q48R SEL29 is a useful RNase H2 enzyme for RNase H2-mediated PCR applications and systems, such as Applicant's rhAmpSeq™ system.

### RNase H2-Mediated SNP Detection and Rare Allele Detection

Owing to their enhanced 3′-mismatch discrimination attributes, the hybrid RNase H2 mutant proteins disclosed herein can be used to detect single nucleotide polymorphism detection and rare allele detection. The use of block-cleavable primers that only form perfect duplexes with the desired SNP-containing nucleic acid templates will be recognized and cleaved by the hybrid RNase H2 mutant proteins, thereby activating the primer:desired nucleic acid template duplexes for primer extension by a DNA polymerase under suitable conditions.

### RNase H2 in Loop-Meditated Isothermal Amplification (LAMP)

Use of RNase H2 in Loop-meditated isothermal amplification (LAMP) is also contemplated herein. The LAMP method of amplification is performed under isothermal conditions, that is, without changes in reaction temperature during cycling. LAMP requires a minimum of four different primers designed to recognize six different regions of the desired amplicon (Notomi, et al. Nucleic Acids Research, 28 (12) (2000)). The amplification reaction depends on the strand displacement activity of the DNA polymerase, usually from Bacillus stearothermophilus (Bst). The products have a structure that consists of a long chain of inverted repeats of the target sequence.

LAMP reactions are prone to formation of primer-dimer products, owing to the large number of primers and the use of the mesophilic DNA polymerase in the method. LAMP also lacks a 5′->3′ exonuclease activity in the amplifying BST polymerase, due to the fact that this activity would destroy the amplification by competing with the essential strand-displacement activity. The use of blocked-cleavable primers and RNase H2 can be employed to reduce or eliminate the detection of primer-dimer signal in LAMP reactions.

In this regard, the hybrid RNase H2 mutant proteins are particularly amendable to enhancing performance of desired products formed in LAMP reactions without attendant production of primer-dimers.

### Applications

In a first aspect, The present invention pertains to a kit for producing an extended primer, comprising at least one container providing a hybrid RNase H2 protein, including a hybrid RNase H2 protein comprising fragments of amino acid sequences from Pyrococcus abyssi (P. a.), Thermococcus kodakarensis (T. kod), and Pyrococcus furiosus organisms; a hybrid RNase H2 protein comprises amino acid residues 26-40 and residues 100-120 of T. kod RNase H2; a hybrid RNase H2 protein is selected from SEQ ID NO: 2 and 3; and a hybrid RNase H2 protein is selected from SEQ ID NO: 14-20.

Finally, the RNase H2 polypeptides of the present invention are amenable for use in the BaseX PCR amplification method, a highly efficient amplification method disclosed in U.S. Pat. No. 10,273,534 (B2), the contents of which are herein incorporated by reference in its entirety.

### EXAMPLES

The present invention is further illustrated by reference to the following Examples. However, it should be noted that these Examples, like the embodiments described above, are illustrative and are not to be construed as restricting the enabled scope of the invention in any way.

### Example 1. Generation of SEL28 and SEL29 Hybrid RNase H2 Proteins via Recombination Reshuffling

Mutant RNase H2 proteins were synthesized using in vitro DNA recombination and directed molecular evolution techniques. Altravax™ Inc. (Sunnyvale, CA) generated a library of 5,500 mutants under the contract agreement with Integrated DNA Technologies. SEL28 and SEL29 mutants were selected in initial screening done in IDT where the mutants exhibited increased mismatch discrimination in an RNase H2 cleavage reaction. The mutants were created in pET-27b(+) plasmid vector inside of Escherichia coli (E. coli) BL21 (DE3). Expressed proteins based on T7 system contain a pelB signal sequence at the N-terminus, mutated RNase H2 genes, a human herpes simplex virus 2 epitope tag, and a six-histidine tag at the C-terminus. E. coli cells were grown in 12 mL of LB Broth with 50 μg/mL kanamycin (Teknova™, Hollister, CA) using 50 mL TPP TubeSpin® Bioreactors (Techno Plastic Products AG, Trasadingen, Switzerland). Expression of RNase H2 was induced using the Overnight Express™ Autoinduction System 1 (MilliporeSigma™, Burlington, MA) at 37° C. for 20 hours. Bacterial expression strains were grown while shaken at 250 rpm using a MaxQ™ 4000 orbital shaker (ThermoFisher Scientific™, Grand Island, NY). Cells were centrifuged at 7,500×g for 10 minutes in ThermoScientific Sorvall™ Legend XTR centrifuge and the supernatant was discarded. The cell paste was stored at −80° C. and resuspended in 0.6 mL of lysis solution consisting of the cell resuspension buffer containing 50 mM NaCl, 40 mM Tris-HCl pH 8.0, 2.5 mM MgCl₂, 0.5 mM CaCl₂, 1× BugBuster® Extraction Reagent (MilliporeSigma™, Burlington, MA), 1× cOmplete™, EDTA-free protease inhibitor cocktail (MilliporeSigma™, Burlington, MA), 0.1 mg/mL of lysozyme (˜600 units, ThermoFisher™ Scientific, Grand Island, NY), and 4 U/mL of Ambion™ DNase I (ThermoFisher™ Scientific, Grand Island, NY). Lysis took 15 minutes at 25° C. while cells were shaken at 120 rpm. Insoluble materials were removed by centrifugation at 16,000×g for 20 minutes. DNase I and native E. coli proteins were denatured at 75° C. for 15 minutes. Insoluble denatured proteins were again removed by centrifugation at 16,000×g for 15 minutes. Capturem™ His-Tagged Miniprep Kit (Takara Bio™, Mountain View, CA) was used for protein purification. Binding columns from the kit were washed with the cell resuspension buffer and supernatants containing RNase H2 were loaded on the columns. Solutions were pulled through columns by centrifugation at 11,000×g for 1 minute. Columns were washed twice with 200 μL of wash buffer (20 mM Na₃PO₄, 150 mM NaCl, pH 7.6) with 20 mM imidazole added. RNase H2 enzymes were eluted with 200 μL of elution buffer (20 mM Na₃PO₄, 500 mM NaCl, 500 mM imidazole, pH 7.6) and dialyzed against 1 L of 2× storage buffer F (pH 8.4, 40 mM Tris-HCl, 0.2 mM EDTA, 200 mM KCl) overnight using D-Tube™ Dialyzer Midi, MWCO 6-8 kDa (MilliporeSigma™, Burlington, MA). Dialysis buffer in the tank was replaced at least once. Samples were recovered from dialysis and mixed in 1:1 volume ratio with mixture of 99.8% (v/v) glycerol and 0.2% (v/v) Triton X-100. These purified and concentrated RNase H2 solutions were stored at −20° C. Their purity was estimated by SDS gel electrophoresis using Any kD™ Mini-PROTEAN® TGX Stain-Free™ Protein Gels (Bio-Rad®, Hercules, CA). RNase H2 enzyme exhibited dominant protein band (>75%) in all purified samples and its position corresponded to expected molecular mass of 28.9 kg/mol compared to Precision Plus Protein™ Unstained Standards (Bio-Rad®, Hercules, CA).

The amino acid sequences of the mutant proteins are shown in Table 1, SEQ ID NOs.: 89 and 90. The sequencing was done using Applied Biosystems BigDye Terminators v3.1 kit. Plasmid DNA was isolated from bacterial strains with Wizard® Plus SV Minipreps DNA Purification System (Promega, Madison, WI) following the manufacturer's protocol. Sequencing data were collected by Applied Biosystems 3130 Genetic Analyzer.

### Example 2. Q48R and A107V SDM Mutants in SEL29 RNase H2 Increase Enzymatic Activity in Thermococcus kodakarensis DNA Polymerase Reaction Buffer Compared to SEL29 RNase H2

(His)₆-tagged mutant SEL28 and SEL29 RNase H2 proteins were generated by site-directed mutagenesis (SDM) techniques (see, e.g., Weiner M. et al., Gene, 151:119-123 (1994)). Primers used for SDM of SEL28 and SEL29 RNase H2 are shown in Table 2, SEQ ID NOs.: 4-13. The mutants were sequence verified, and protein was expressed in E. coli using standard methods. Purification was done by affinity purification over a charged Ni²⁺ column, as described earlier (Dobosy et al., 2011, and U.S. Pat. No. 8,911,948 B2). The amino acid sequences of the mutant proteins are shown in Table 3, SEQ ID NOs.: 2-3, 14-20.

TABLE 2
SDIM primers for mutagenesis of
the RNase H2 enzymes.

| | SEQ | Specific | |
|---|---|---|---|
| Primer | ID | AA | |
| name | NO.: | changes | Sequence¹ |
| P13S | 4 | P13S | GGTGCAGATGAAGCTGGTCG |
| SEL28 | | | TGGTTCTGTTATTGGTCCGC |
| Forward Oligo | | | TGGTTATTGTTGCT |
| P13S | 5 | P13S | AGCAACAATAACCAGCGGAC |
| SEL28 | | | CAATAACAGAACCACGACCA |
| Reverse Oligo | | | GCTTCATCTGCACC |
| A107V | 6 | A107V | AAGCCGGATGTTATTTACGC |
| SEL28 | | | TGATGCCGTAGATGTTGATG |
| Forward Oligo | | | AAGAACGTTTCGCTAGA |
| A107V | 7 | A107V | TCTAGCGAAACGTTCTTCAT |
| SEL28 | | | CAACATCTACGGCATCAGCG |
| Reverse Oligo | | | TAAATAACATCCGGCTT |
| P13S | 8 | P13S | GGTATAGATGAAGCTGGTCG |
| SEL29 | | | TGGTTCTGCTATTGGTCCGC |
| Forward Oligo | | | TGGTTATTGTTGGT |
| P13S | 9 | P13S | AGCAACAATAACCAGCGGAC |
| SEL29 | | | CAATAGCAGAACCACGACCA |
| Reverse Oligo | | | GCTTCATCTATACC |
| Q48R | 10 | Q48R | AGACTCCAAAAAGCTGACCC |
| SEL29 | | | CGGCCGCCCGTGAAAAACTG |
| Forward Oligo | | | TTCGATGAAATCG |
| Q48R | 11 | Q48R | CGATTTCATCGAACAGTTTT |
| SEL29 | | | TCACGGCGCGCCGGGGTCAG |
| Reverse Oligo | | | CTTTTTGGAGTCT |
| A107V | 12 | A107V | AAGCCGGATGTTATTTACGC |
| SEL29 | | | TGATGCCGTAGATGTTGATG |
| Forward Oligo | | | AAGAACGTTTCGCTAGA |
| A107V | 13 | A107V | TCTAGCGAAACGTTCTTCAT |
| SEL29 | | | CAACATCTACGGCATCAGCG |
| Reverse Oligo | | | TAAATAACATCCGGCTT |

¹All bases are DNA. Characters shown in bold and underlined are the mutagenic nucleotides.

TABLE 3
Amino acid sequences for the RNase H2 proteins.

| Mut | SEQ | | |
|---|---|---|---|
| ID | ID | Specific AA | |
| # | NO.: | changes | Sequence¹ |
| N/A | 1 | WT P.a. | MKVAGADEAGRGPVIGPLVIVAAVVEEDK |
| | | RNase H2 | IRSLTKLGVKDSKQLTPAQREKLFDEIVKV |
| | | | LDDYSVVIVSPQDIDGRKGSMNELEVENFV |
| | | | KALNSLKVKPEVIYIDSADVKAERFAENIRS |
| | | | RLAYEAKVVAEHKADAKYEIVSAASILAK |
| | | | VIRDREIEKLKAEYGDFGSGYPSDPRTKKW |
| | | | LEEWYSKHGNFPPIVRRTWDTAKKIEEKFK |
| | | | RAQLTLDNFLKRFRNKLAAALEIKRASQPEL |
| | | | APEDPEDVEHHHHHH |
| 1 | 2 | SEL28 RNase | MKVAGADEAGRGPVIGPLVIVAAVVDENS |
| | | H2 | LPKLEELKVRDSKKLTPKRREKLFDEIVKV |
| | | | LDDYSVVIVSPQDIDGRKGSMNELEVENFV |
| | | | KALNSLKVKPDVIYADAADVDEERFAREL |
| | | | GERLNFEAEVVAKHKADDIFPVVSAASILA |
| | | | KVIRDREIEKLKAEYGDFGSGYPSDPRTKK |
| | | | WLEEWYSKHGNFPPIVRRTWDTAKKIEEK |
| | | | FKRAQLTLDKFFKKPKLAAALEIKRASQPEL |
| | | | APEDPEDVEHHHHHH |
| 2 | 3 | SEL29 RNase | MKVAGIDEAGRGPAIGPLVIVAAVVDENSL |
| | | H2 | PKLEELKVRDSKKLTPAQREKLFDEIVKVL |
| | | | DDYSVVIVSPQDIDGRKGSMNELEVENFVK |
| | | | ALNSLKVKPDVIYADAADVDEERFARELG |
| | | | ERLNFEAKVVAEHKADAKYEIVSAASILAK |
| | | | VIRDREIEKLKAEYGDFGSGYPSDPRTRAFL |
| | | | ENYYREHGEFPPIVRKGWKTLKKIAEKVES |
| | | | EKKAEERQATLDRYFRKVKLAAALEIKRASQ |
| | | | PELAPEDPEDVEHHHHHH |
| 3 | 14 | P13S SEL28 | MKVAGADEAGRGSVIGPLVIVAAVVDENS |
| | | RNase H2 | LPKLEELKVRDSKKLTPKRREKLFDEIVKV |
| | | | LDDYSVVIVSPQDIDGRKGSMNELEVENFV |
| | | | KALNSLKVKPDVIYADAADVDEERFAREL |
| | | | GERLNFEAEVVAKHKADDIFPVVSAASILA |
| | | | KVIRDREIEKLKAEYGDFGSGYPSDPRTKK |
| | | | WLEEWYSKHGNFPPIVRRTWDTAKKIEEK |
| | | | FKRAQLTLDKFFKKPKLAAALEIKRASQPEL |
| | | | APEDPEDVEHHHHHH |
| 4 | 15 | A107V | MKVAGADEAGRGPVIGPLVIVAAVVDENS |
| | | SEL28 RNase | LPKLEELKVRDSKKLTPKRREKLFDEIVKV |
| | | H2 | LDDYSVVIVSPQDIDGRKGSMNELEVENFV |
| | | | KALNSLKVKPDVIYADAVDVDEERFAREL |
| | | | GERLNFEAEVVAKHKADDIFPVVSAASILA |
| | | | KVIRDREIEKLKAEYGDFGSGYPSDPRTKK |
| | | | WLEEWYSKHGNFPPIVRRTWDTAKKIEEK |
| | | | FKRAQLTLDKFFKKPKLAAALEIKRASQPEL |
| | | | APEDPEDVEHHHHHH |
| 5 | 16 | P13S/A107V | MKVAGADEAGRGSVIGPLVIVAAVVDENS |
| | | SEL28 RNase | LPKLEELKVRDSKKLTPKRREKLFDEIVKV |
| | | H2 | LDDYSVVIVSPQDIDGRKGSMNELEVENFV |
| | | | KALNSLKVKPDVIYADAVDVDEERFAREL |
| | | | GERLNFEAEVVAKHKADDIFPVVSAASILA |
| | | | KVIRDREIEKLKAEYGDFGSGYPSDPRTKK |
| | | | WLEEWYSKHGNFPPIVRRTWDTAKKIEEK |
| | | | FKRAQLTLDKFFKKPKLAAALEIKRASQPEL |
| | | | APEDPEDVEHHHHHH |
| 6 | 17 | P13S SEL29 | MKVAGIDEAGRGSAIGPLVIVAAVVDENSL |
| | | RNase H2 | PKLEELKVRDSKKLTPAQREKLFDEIVKVL |
| | | | DDYSVVIVSPQDIDGRKGSMNELEVENFVK |
| | | | ALNSLKVKPDVIYADAADVDEERFARELG |
| | | | ERLNFEAKVVAEHKADAKYEIVSAASILAK |
| | | | VIRDREIEKLKAEYGDFGSGYPSDPRTRAFL |
| | | | ENYYREHGEFPPIVRKGWKTLKKIAEKVES |
| | | | EKKAEERQATLDRYFRKVKLAAALEIKRASQ |
| | | | PELAPEDPEDVEHHHHHH |
| 7 | 18 | Q48R SEL29 | MKVAGIDEAGRGPAIGPLVIVAAVVDENSL |
| | | RNase H2 | PKLEELKVRDSKKLTPARREKLFDEIVKVL |
| | | | DDYSVVIVSPQDIDGRKGSMNELEVENFVK |
| | | | ALNSLKVKPDVIYADAADVDEERFARELG |
| | | | ERLNFEAKVVAEHKADAKYEIVSAASILAK |
| | | | VIRDREIEKLKAEYGDFGSGYPSDPRTRAFL |
| | | | ENYYREHGEFPPIVRKGWKTLKKIAEKVES |
| | | | EKKAEERQATLDRYFRKVKLAAALEIKRASQ |
| | | | PELAPEDPEDVEHHHHHH |
| 8 | 19 | A107V | MKVAGIDEAGRGPAIGPLVIVAAVVDENSL |
| | | SEL29 RNase | PKLEELKVRDSKKLTPAQREKLFDEIVKVL |
| | | H2 | DDYSVVIVSPQDIDGRKGSMNELEVENFVK |
| | | | ALNSLKVKPDVIYADAVDVDEERFARELG |
| | | | ERLNFEAKVVAEHKADAKYEIVSAASILAK |
| | | | VIRDREIEKLKAEYGDFGSGYPSDPRTRAFL |
| | | | ENYYREHGEFPPIVRKGWKTLKKIAEKVES |
| | | | EKKAEERQATLDRYFRKVKLAAALEIKRASQ |
| | | | PELAPEDPEDVEHHHHHH |
| 9 | 20 | P13S/A107V | MKVAGIDEAGRGSAIGPLVIVAAVVDENSL |
| | | SEL29 RNase | PKLEELKVRDSKKLTPAQREKLFDEIVKVL |
| | | H2 | DDYSVVIVSPQDIDGRKGSMNELEVENFVK |
| | | | ALNSLKVKPDVIYADAVDVDEERFARELG |
| | | | ERLNFEAKVVAEHKADAKYEIVSAASILAK |
| | | | VIRDREIEKLKAEYGDFGSGYPSDPRTRAFL |
| | | | ENYYREHGEFPPIVRKGWKTLKKIAEKVES |
| | | | EKKAEERQATLDRYFRKVKLAAALEIKRASQ |
| | | | PELAPEDPEDVEHHHHHH |

¹Location of mutations are shown in bold and underlined. Ending extension and (His)₆-tag is shown in italics.

To determine whether P13S, A107V, and P13S/A107V SEL28 RNase H2 or P13S, Q48R, A107V, or P13S/A107V SEL29 RNase H2 have increased activity in KOD DNA polymerase reaction buffer with rhPCR compared to WT P. a. RNase H2, a quantitative rhPCR assay targeting the rs4939827 SNP in SMAD7 was designed. This SNP has been utilized in the past (Dobosy et al., 2011, and U.S. Pat. No. 8,911,948 B2) to characterize rhPCR efficiency and specificity, and its response under differing conditions is well understood. The primers used in this assay are shown in Table 4, SEQ 21-23. Assays were run in 10 μL reaction volumes. Thermal cycling and data collection were run on a CFX384® Real Time System (Bio-Rad®, Hercules, CA). Briefly, either 200 nM (2 pmol) of the blocked forward primer (SEQ ID NO.: 22) and 200 nM (2 pmol) of the unblocked reverse primer (SEQ ID NO.: 23), or 200 nM (2 pmol) of the unblocked forward primer (SEQ ID NO.: 21) and 200 nM (2 pmol) of the unblocked reverse primer (SEQ ID NO.: 23) were mixed into 1× of internal KOD buffer (ROKStar buffer v2.0 or v1.66) (IDT, Coralville, IA) with 2.5 mM (total) MgSO₄, 0.2 mM (each) dNTPs (MilliporeSigma™, Burlington, MA), and 0.5× EvaGreen® Dye (Biotium Inc., Fremont, CA). RNase H2 dilution buffer (IDT, Coralville, IA) or 21 fmol of WT P. a. (SEQ ID NO.: 1), SEL28 (SEQ ID NO.: 2), SEL29 (SEQ ID NO.: 3), P13S SEL28 (SEQ ID NO.: 14), A107V SEL28 (SEQ ID NO.: 15), P13S/A107V SEL28 (SEQ ID NO.: 16), P13S SEL29 (SEQ ID NO.: 17), Q48R SEL29 (SEQ ID NO.: 18), A107V SEL29 (SEQ ID NO.: 19), or P13S/A107V SEL29 (SEQ ID NO.: 20) RNase H2 enzyme was added to each reaction. 10 ng of genomic cell line DNA (cell line NA12878, Coriell Institute for Medical Research, Camden, NJ), representing a homozygous genotype at the rs4939827 SNP was added to each reaction. Reactions were performed in triplicate, and the results averaged. Reactions were cycled under the following conditions: 95° C.^{3:00}->(95° C.^{0:10}->60° C.^{0:30})×75. Fluorescence data for the intercalated EvaGreen® were collected after each extension time point. After the assay was completed, the data were analyzed, and C_{q} values were calculated using the automatic calling function of the Bio-Rad CFX Manager® software. The results are presented in Table 5.

TABLE 4
Sequences and SEQ IDs for the primers used in the
experiment described in Example 2.

| SEQ | | |
|---|---|---|
| ID | | |
| NO.: | Name | Sequence¹ |
| 21 | rs4939827 For | CAGCCTCATCCAAAAGAGGAAA |
| | unblocked | |
| 22 | rs4939827 For | CAGCCTCATCCAAAAGAGGAAAcAGGAG-X |
| | blocked | |
| 23 | rs4939827 Rev | CTCACTCTAAACCCCAGCATT |
| | unblocked | |

¹DNA is uppercase, RNA is lowercase. X = Proprietary blocker group resistant to exonuclease.

TABLE 5
C_{q}, ΔC_{q}, and ΔΔC_{q} values for the experiments in Example 2.

| | Unblocked | Blocked | | |
|---|---|---|---|---|
| RNase H2 in ROKstar | (SEQ ID | (SEQ ID | | ²ΔΔC_{q} (WT- |
| buffer v2.0 | NO.: 21) | NO.: 22) | ¹ΔC_{q} | Mutant) |
| None | 24.2 | >75.0 | >50.8 | n/a |
| WT (P.a.) | 24.3 | 36.7 | 12.4 | n/a |
| SEL28 (Mut ID 1) | 24.2 | 41.3 | 17.1 | 0 |
| P13S SEL28 | 24.3 | >75.0 | >50.8 | <−33.7 |
| (Mut ID 3) | | | | |
| A107V SEL28 | 24.1 | 44.1 | 20.0 | −2.9 |
| (Mut ID 4) | | | | |
| P13S/A107V SEL28 | 24.1 | 45.7 | 21.5 | −4.5 |
| (Mut ID 5) | | | | |
| SEL29 (Mut ID 2) | 24.2 | 48.9 | 24.6 | 0 |
| P13S SEL29 | 23.6 | >75.0 | >51.4 | <−26.8 |
| (Mut ID 6) | | | | |
| Q48R SEL29 | 24.2 | 41.4 | 17.2 | 7.4 |
| (Mut ID 7) | | | | |
| A107V SEL29 | 24.1 | 38.0 | 13.9 | 10.7 |
| (Mut ID 8) | | | | |
| P13S/A107V SEL29 | 24.4 | 51.4 | 26.9 | −2.3 |
| (Mut ID 9) | | | | |
| | Unblocked | Blocked | | |
| RNase H2 in ROKstar | (SEQ ID | (SEQ ID | | ²ΔΔC_{q} (WT- |
| buffer v1.66 | NO.: 21) | NO.: 22) | ¹ΔC_{q} | Mutant) |
| None | 24.7 | >75.0 | >50.3 | n/a |
| WT (P.a.) | 24.6 | 45.7 | 21.1 | n/a |
| SEL28 (Mut ID 1) | 25.0 | 55.6 | 30.6 | 0 |
| P13S SEL28 | 24.6 | >75.0 | >50.4 | <−19.8 |
| (Mut ID 3) | | | | |
| A107V SEL28 | 24.8 | 60.6 | 35.8 | −5.2 |
| (Mut ID 4) | | | | |
| P13S/A107V SEL28 | 24.7 | 56.4 | 31.8 | −1.2 |
| (Mut ID 5) | | | | |
| SEL29 (Mut ID 2) | 24.1 | 54.5 | 30.3 | 0 |
| P13S SEL29 | 24.9 | >75.0 | >50.1 | <−19.8 |
| (Mut ID 6) | | | | |
| Q48R SEL29 | 25.0 | 50.4 | 25.4 | 4.9 |
| (Mut ID 7) | | | | |
| A107V SEL29 | 24.9 | 46.9 | 21.9 | 8.4 |
| (Mut ID 8) | | | | |
| P13S/A107V SEL29 | 24.7 | 57.4 | 32.8 | −2.4 |
| (Mut ID 9) | | | | |

¹ΔC_{q} values were calculated as the difference between the C_{q} value for the blocked primer (SEQ ID NO.: 22) and the C_{q} value for the unblocked primer (SEQ ID NO.: 21).
²ΔΔC_{q} values were calculated as the difference between the ΔC_{q} value for the background SEL28 or SEL29 RNase H2 and the ΔC_{q} value for the mutant SEL28 or SEL29 RNase H2.

These data show that Q48R SEL29 and A107V SEL29 RNase H2 have increased enzymatic activity in Thermococcus kodakarensis DNA polymerase reaction buffer compared to the background SEL29 RNase H2, but P13S SEL29 RNase H2 and P13S/A107V RNase H2 does not. In addition, P13S SEL28, A107V SEL28, and P13S/A107V RNase H2 do not increase enzymatic activity in Thermococcus kodakarensis DNA polymerase reaction buffer compared to the background SEL28 RNase H2. Without the addition of any RNase H2, the blocked primers are not cleaved and thus cannot support PCR. Use of a proprietary exonuclease-resistant blocked primer is necessary to prevent deblocking of the primer by the high-fidelity DNA polymerase. WT (P. a.) RNase H2 is capable of cleaving the blocked primers, but has a delay in amplification, resulting in a ΔC_{q} of 12.4 cycles in ROKstar buffer v2.0 and 21.1 cycles in ROKstar buffer v1.66 compared to the unblocked primers. The ΔC_{q} quantification of the delay in amplification increases to 17.1 cycles for SEL28 RNase H2 and 24.6 cycles for SEL29 RNase H2 in ROKstar buffer v2.0 and increases to 30.6 cycles for SEL28 RNase H2 and 30.3 cycles for SEL29 RNase H2 in ROKstar buffer v1.66. The ΔC_{q} quantification of the delay in amplification decreases with Q48R SEL29 RNase H2 (17.2 cycles in ROKstar buffer v2.0 and 25.4 cycles in ROKstar buffer v1.66), and A107V RNase H2 (13.9 cycles in ROKstar buffer v2.0 and 21.9 cycles in ROKstar buffer v1.66). P13S/A107V SEL29 RNase H2 increases the ΔC_{q} quantification of the delay in amplification to 26.9 cycles in ROKstar buffer v2.0 and 32.8 cycles in ROKstar buffer v1.66. P13S SEL28 and P13S SEL29 RNase H2 have no apparent activity in either ROKstar buffer v2.0 or v1.66. In addition, P13S/A107V SEL28 RNase H2 increases the ΔC_{q} quantification of the delay in amplification to 21.5 cycles in ROKstar buffer v2.0 and 31.8 cycles in ROKstar buffer v1.66. A107V SEL28 RNase H2 increases the ΔC_{q} quantification of the delay in amplification to 20.0 cycles in ROKstar buffer v2.0 and 35.8 cycles in ROKstar buffer v1.66. These results contrast with the mutations in P. a. RNase H2, as Q48R, A107V, and P13S/A107V P. a. RNase H2 all have increased enzymatic activity in Thermococcus kodakarensis DNA polymerase reaction buffer, and P13S P. a. RNase H2 had lower, yet measurable, enzymatic activity in Thermococcus kodakarensis DNA polymerase reaction buffer. Q48R SEL29 and A107V SEL29 RNase H2 improve enzymatic activity—though to differing degrees—when used with Thermococcus kodakarensis DNA polymerase reaction buffer.

### Example 3. Q48R SEL29 and A107V SEL29 RNase H2 Increase Mismatch Discrimination Compared to WT RNase H2 When the Mismatch Is Placed Opposite of the RNA

The specific activity of the enzyme was determined using a fluorescence-based kinetic assay. The sequences for the DNA substrates are shown in Table 6, SEQ ID NOs.: 24-25. The substrate is a DNA hairpin with a matched RNA base within the double-stranded region. Attached to the 3′ end of the probe is a 6-FAM (6-carboxyfluorescein); attached to the 5′ end of the probe is an Iowa Black® FQ (SEQ ID NO.: 24). The fluorescence of the 6-FAM is quenched by the Iowa Black® FQ moiety in the intact hairpin probe. RNase H2 cleaves 5′ of the RNA base and releases the 3′ end of the probe with the 6-FAM. Thus, the fluorescence of the 6-FAM is no longer be quenched and can fluoresce. A DNA hairpin with the RNA base but without the fluorophore or the quencher was used as a competitor (SEQ ID NO.: 25). Assays were performed in 10 μL reaction volumes. Data collection was carried out with a LightCycler® 480 II (Roche Life Science, Indianapolis, IN). Briefly, 1× of the rhAmp™ Backbone v3 was used in combination with 200 nM (2 pmol) of labeled hairpin (SEQ ID NO.: 24) and 10 μM (100 pmol) of competitor hairpin (SEQ ID NO.: 25). 0.5, 1.0, 2.0, or 5.0 fmol of WT P. a. RNase H2 (SEQ ID NO.: 1) or mutant RNase H2 (SEQ ID NOs.: 18-19) were added to each reaction. Reactions were initially kept at 4° C. to prevent cleavage of substrate prior to starting the assay. Samples were run at 65° C. The fluorescence excitation wavelength was 483 nm; the fluorescence emission wavelength was 533 nm. Fluorescence intensities were collected every 13.75 seconds for 135 minutes. The initial velocities and the velocity per femtomole for each reaction were calculated. The velocities per femtomole for each mutant was normalized to the values of WT P. a. RNase H2, which was previously determined to have a specific activity of 17 Units per μg of enzyme. Q48R SEL29 RNase H2 has a specific activity of 46.03 Units per μg of enzyme, and A107V SEL29 RNase H2 has a specific unit activity of 7.77 Units per μg of enzyme.

TABLE 6
Sequences and SEQ IDs for the DNA hairpins to
determine RNase H2 unit activity.

| SEQ | | |
|---|---|---|
| ID | | |
| NO.: | Name | Sequence¹ |
| 24 | Dye-quencher | Q-TATAAGCTACCAGCATGGTTTTT |
| | labeled hairpin | CCATGCTGGTAGcTTATA-F |
| 25 | Native competitor | TATAAGCTACCAGCATGGTTTTT |
| | hairpin | CCATGCTGGTAGcTTATA |

¹DNA is uppercase, RNA is lowercase. Q = Iowa Black ® FQ. F = 6-FAM (Fluorescein).

To fully determine whether these mutant RNase H2 enzymes can improve mismatch discrimination with the mismatch directly opposite of the RNA base, a synthetic, quantitative rhPCR assay was utilized as described previously (Dobosy et al, 2011, and U.S. Pat. No. 8,911,948 B2). This assay can directly compare the effects of each specific single-base mismatch compared to the perfect match. The primers used in this assay are shown in Table 7, SEQ ID NOs.: 26-31. Assays were run in 10 μL reaction volumes. Thermal cycling and data collection were run on a CFX384® Real Time System (Bio-Rad®, Hercules, CA). Briefly, either 200 nM (2 pmol) of a blocked reverse primer (SEQ ID NOs.: 28-31) and 200 nM (2 pmol) of the unblocked forward primer (SEQ ID NO.: 26), or 200 nM (2 pmol) of the unblocked reverse primer (SEQ ID NO.: 27) and 200 nM (2 pmol) of the unblocked forward primer (SEQ ID NO.: 26) were mixed into 1× iQ™ SYBR® Green Supermix® (Bio-Rad®, Hercules, CA). 5 mU of WT P. a. (SEQ ID NO.: 1), 40 mU of Q48R SEL29 (SEQ ID NO.: 18), or 5 mU of A107V SEL29 (SEQ ID NO.: 19) RNase H2 enzyme was added to each reaction. 20,000 copies of each synthetic target sequence (SEQ ID NOs.: 32-35), each with a different nucleotide directly opposite the RNA base, were added to each reaction. Reactions were performed in triplicate, and results averaged. Reactions were cycled under the following conditions: 95° C.^{3:00}→(95° C.^{0:10}→60° C.^{0:30})×75. Fluorescence data for the intercalated SYBR® Green were collected after each extension time point. After the assay was completed, the data were analyzed, and the average C_{q} and ΔC_{q} values for each pair-wise combination were calculated. The results are presented in Tables 8 and 9.

TABLE 7
Sequences and SEQ IDs for the primers and
templates used in the experiment described in
Example 2.

| SEQ | | |
|---|---|---|
| ID | | |
| NO.: | Name | Sequence¹ |
| 26 | Syn For | AGCTCTGCCCAAAGATTACCCTG |
| | unblocked | |
| 27 | Syn Rev | CTGAGCTTCATGCCTTTACTGT |
| | unblocked | |
| 28 | Syn Rev rA | CTGAGCTTCATGCCTTTACTGTaCCCCC-X |
| | blocked | |
| 29 | Syn Rev rC | CTGAGCTTCATGCCTTTACTGTcCCCCC-X |
| | blocked | |
| 30 | Syn Rev rG | CTGAGCTTCATGCCTTTACTGTgCCCCC-X |
| | blocked | |
| 31 | Syn Rev rU | CTGAGCTTCATGCCTTTACTGTuCCCCC-X |
| | blocked | |
| 32 | Syn A | AGCTCTGCCCAAAGATTACCCTGACAGCTAAGT |
| | template | GGCAGTGGAAGTTGGCCTCAGAAGTAGTGGCCA |
| | | GCTGTGTGTCGGGGAACAGTAAAGGCATGAAGC |
| | | TCAG |
| 33 | Syn C | AGCTCTGCCCAAAGATTACCCTGACAGCTAAGT |
| | template | GGCAGTGGAAGTTGGCCTCAGAAGTAGTGGCCA |
| | | GCTGTGTGTCGGGGCACAGTAAAGGCATGAAGC |
| | | TCAG |
| 34 | Syn G | AGCTCTGCCCAAAGATTACCCTGACAGCTAAGT |
| | template | GGCAGTGGAAGTTGGCCTCAGAAGTAGTGGCCA |
| | | GCTGTGTGTCGGGGGACAGTAAAGGCATGAAGC |
| | | TCAG |
| 35 | Syn U | AGCTCTGCCCAAAGATTACCCTGACAGCTAAGT |
| | template | GGCAGTGGAAGTTGGCCTCAGAAGTAGTGGCCA |
| | | GCTGTGTGTCGGGGTACAGTAAAGGCATGAAGC |
| | | TCAG |

¹DNA is uppercase, RNA is lowercase. X = C3 spacer (propanediol) blocker group. Location of the mismatch is shown in bold and underlined in the synthetic template.

TABLE 8
C_{q} values for the experiment described in Example 2.

| | | Reverse | C_{q} for | C_{q} for | C_{q} for | C_{q} for |
|---|---|---|---|---|---|---|
| | Reverse | primer | Syn A | Syn C | Syn G | Syn T |
| RNase H2 | primer | SEQ ID | tem- | tem- | tem- | tem- |
| enzyme | name | NO.: | plate | plate | plate | plate |
| WT (P.a.) | Syn Rev | 27 | 26.2 | 27.5 | 26.8 | 27.7 |
| RNase H2 | unblocked | | | | | |
| | Syn Rev rA | 28 | 28.8 | 28.6 | 32.7 | 28.2 |
| | blocked | | | | | |
| | Syn Rev rC | 29 | 26.8 | 29.4 | 27.2 | 28.6 |
| | blocked | | | | | |
| | Syn Rev rG | 30 | 37.9 | 27.6 | 35.6 | 33.5 |
| | blocked | | | | | |
| | Syn Rev rU | 31 | 26.4 | 32.3 | 36.6 | 28.8 |
| | blocked | | | | | |
| Q48R | Syn Rev | 27 | 26.7 | 27.5 | 27.0 | 28.1 |
| SEL29 | unblocked | | | | | |
| RNase H2 | Syn Rev rA | 28 | 37.1 | 39.9 | 41.5 | 29.2 |
| (Mut ID 7) | blocked | | | | | |
| | Syn Rev rC | 29 | 35.5 | 51.6 | 27.6 | 40.8 |
| | blocked | | | | | |
| | Syn Rev rG | 30 | 37.1 | 32.7 | 39.1 | 43.7 |
| | blocked | | | | | |
| | Syn Rev rU | 31 | 26.3 | 46.6 | 40.3 | 44.2 |
| | blocked | | | | | |
| WT (P.a.) | Syn Rev | 27 | 26.1 | 26.1 | 26.8 | 28.5 |
| RNase H2 | unblocked | | | | | |
| | Syn Rev rA | 28 | 28.5 | 34.7 | 36.9 | 26.9 |
| | blocked | | | | | |
| | Syn Rev rC | 29 | 26.8 | 37.4 | 26.9 | 30.9 |
| | blocked | | | | | |
| | Syn Rev rG | 30 | 37.4 | 28.7 | 37.6 | 39.6 |
| | blocked | | | | | |
| | Syn Rev rU | 31 | 26.3 | 41.9 | 37.5 | 31.3 |
| | blocked | | | | | |
| A107V | Syn Rev | 27 | 26.3 | 26.9 | 26.7 | 26.8 |
| SEL29 | unblocked | | | | | |
| RNase H2 | Syn Rev rA | 28 | 39.4 | 41.6 | 38.4 | 27.4 |
| (Mut ID 8) | blocked | | | | | |
| | Syn Rev rC | 29 | 38.9 | 53.0 | 27.2 | 44.6 |
| | blocked | | | | | |
| | Syn Rev rG | 30 | 40.2 | 31.6 | 37.7 | 40.7 |
| | blocked | | | | | |
| | Syn Rev rU | 31 | 26.5 | 46.9 | 36.9 | 40.5 |
| | blocked | | | | | |
| WT (P.a.) | Syn Rev | 27 | 25.2 | 26.3 | 25.2 | 25.6 |
| RNase H2 | unblocked | | | | | |
| | Syn Rev rA | 28 | 30.2 | 26.1 | 35.0 | 24.8 |
| | blocked | | | | | |
| | Syn Rev rC | 29 | 25.2 | 26.3 | 25.2 | 25.6 |
| | blocked | | | | | |
| | Syn Rev rG | 30 | 36.0 | 25.3 | 35.6 | 31.6 |
| | blocked | | | | | |
| | Syn Rev rU | 31 | 24.5 | 28.7 | 34.5 | 25.9 |
| | blocked | | | | | |
| SEL29 | Syn Rev | 27 | 24.4 | 24.6 | 24.9 | 24.7 |
| RNase H2 | unblocked | | | | | |
| (Mut ID 2)¹ | Syn Rev rA | 28 | 37.2 | 38.7 | 37.2 | 25.1 |
| | blocked | | | | | |
| | Syn Rev rC | 29 | 37.0 | 39.6 | 25.3 | 36.6 |
| | blocked | | | | | |
| | Syn Rev rG | 30 | 37.9 | 26.0 | 36.7 | 38.6 |
| | blocked | | | | | |
| | Syn Rev rU | 31 | 26.3 | 39.6 | 36.4 | 37.9 |
| | blocked | | | | | |

TABLE 9
ΔC_{q} values for the experiment described in Example 2.

| | | Reverse | | | | |
|---|---|---|---|---|---|---|
| | Reverse | primer | ΔC_{q} for | ΔC_{q} for | ΔC_{q} for | ΔC_{q} for |
| RNase H2 | primer | SEQ ID | Syn A | Syn C | Syn G | Syn T |
| enzyme | name | NO.: | template | template | template | template |
| WT (P.a.) | Syn Rev rA | 28 | 0.6 | 0.3 | 4.5 | 0 |
| RNase H2 | blocked | | | | | |
| | Syn Rev rC | 29 | −0.4 | 2.2 | 0 | 1.5 |
| | blocked | | | | | |
| | Syn Rev rG | 30 | 10.3 | 0 | 8.0 | 5.9 |
| | blocked | | | | | |
| | Syn Rev rU | 31 | 0 | 6.0 | 10.2 | 2.4 |
| | blocked | | | | | |
| Q48R | Syn Rev rA | 28 | 7.9 | 10.7 | 12.3 | 0 |
| SEL29 | blocked | | | | | |
| RNase H2 | Syn Rev rC | 29 | 8.0 | 24.1 | 0 | 13.2 |
| (Mut ID 7) | blocked | | | | | |
| | Syn Rev rG | 30 | 4.5 | 0 | 6.5 | 11.0 |
| | blocked | | | | | |
| | Syn Rev rU | 31 | 0 | 20.3 | 14.0 | 17.9 |
| | blocked | | | | | |
| WT (P.a.) | Syn Rev rA | 28 | 1.5 | 7.7 | 9.9 | 0 |
| RNase H2 | blocked | | | | | |
| | Syn Rev rC | 29 | −0.1 | 10.5 | 0 | 4.0 |
| | blocked | | | | | |
| | Syn Rev rG | 30 | 8.7 | 0 | 8.9 | 10.9 |
| | blocked | | | | | |
| | Syn Rev rU | 31 | 0 | 15.6 | 11.2 | 5.1 |
| | blocked | | | | | |
| A107V | Syn Rev rA | 28 | 12.0 | 14.2 | 11.0 | 0 |
| SEL29 | blocked | | | | | |
| RNase H2 | Syn Rev rC | 29 | 11.7 | 25.7 | 0 | 17.4 |
| (Mut ID 8) | blocked | | | | | |
| | Syn Rev rG | 30 | 8.7 | 0 | 6.2 | 9.1 |
| | blocked | | | | | |
| | Syn Rev rU | 31 | 0 | 20.4 | 10.4 | 14.0 |
| | blocked | | | | | |
| WT (P.a.) | Syn Rev rA | 28 | 5.4 | 1.4 | 10.3 | 0 |
| RNase H2 | blocked | | | | | |
| | Syn Rev rC | 29 | 0.0 | 1.0 | 0 | 0.3 |
| | blocked | | | | | |
| | Syn Rev rG | 30 | 10.7 | 0 | 10.3 | 6.3 |
| | blocked | | | | | |
| | Syn Rev rU | 31 | 0 | 4.2 | 10.0 | 1.4 |
| | blocked | | | | | |
| SEL29 | Syn Rev rA | 28 | 12.0 | 13.5 | 12.1 | 0 |
| RNase H2 | blocked | | | | | |
| (Mut ID 2) | Syn Rev rC | 29 | 11.8 | 14.3 | 0 | 11.3 |
| | blocked | | | | | |
| | Syn Rev rG | 30 | 11.9 | 0 | 10.7 | 12.6 |
| | blocked | | | | | |
| | Syn Rev rU | 31 | 0 | 13.3 | 10.1 | 11.6 |
| | blocked | | | | | |

ΔC_{q} values from Table 9 were calculated as the difference between the C_{q} value for the nucleotide-specific primer for each template and the C_{q} value for the perfect match template.

These data show a massive increase in mismatch discrimination when the mismatch is opposite of rC for Q48R SEL29 and A107V SEL29 RNase H2 (˜14.0 cycles and ˜13.5 cycles respectively). In particular, there is a large increase in mismatch discrimination for the rC:C pair for Q48R SEL29 and A107V SEL29 RNase H2 (21.9 cycles and 15.2 cycles respectively). When the mismatch is opposite of rU, there is a significant increase in mismatch discrimination for Q48R SEL29 RNase H2 (˜11.2 cycles), but a less significant increase for A107V SEL29 RNase H2 (˜4.3 cycles). When the mismatch is opposite of rA, there is a significant change in mismatch discrimination for Q48R SEL29 and A107V SEL29 RNase H2 (˜8.5 cycles and ˜6.0 cycles respectively). When the mismatch is opposite of rG, there is little change in mismatch discrimination; this lack of change of mismatch discrimination is less crucial, as the mismatch discrimination was already rather good for rG mismatches using WT RNase H2. These increases in mismatch discrimination for Q48R SEL29 and A107V SEL29 RNase H2 are similar to those shown for the background SEL29 RNase H2. Both mutations improve mismatch discrimination when the mismatch is located directly opposite of the RNA base, but to different degrees and with different specificities.

### Example 4. Q48R SEL29 and A107V SEL29 RNase H2 Increase Mismatch Discrimination Compared to WT RNase H2 When the Mismatch Is Placed 5′ of the RNA

To determine the degree of mismatch discrimination when the mismatch is located 5′ of the RNA nucleotide using the Q48R SEL29 and A107V SEL29 RNase H2 enzymes, an assay targeting rs113488022—the V600E SNP in the human BRAF gene—was designed with the SNP located immediately 5′ of the RNA. The primers used in this assay are shown in Table 10, SEQ ID NOs.: 36-39. Assays were run in 10 μL reaction volumes. Thermal cycling and data collection were run on a CFX384® Real Time System (Bio-Rad®, Hercules, CA). Briefly, either 200 nM (2 pmol) of a blocked forward primer (SEQ ID NOs.: 38 or 39) and 200 nM (2 pmol) of the unblocked reverse primer (SEQ ID NO.: 37), or 200 nM (2 pmol) of the unblocked forward primer (SEQ ID NO.: 36) and 200 nM (2 pmol) of the unblocked reverse primer (SEQ ID NO.: 37) were mixed into 1× iQ™ SYBR® Green Supermix®. 5 mU or 10 mU of WT (SEQ ID NO.: 1) P. a. RNase H2 enzyme, 5 mU or 10 mU of SEL29 (SEQ ID NO.: 3) RNase H2 enzyme, 20 mU or 40 mU of Q48R SEL29 (SEQ ID NO.: 18) RNase H2 enzyme, or 5 mU or 10 mU of A107V SEL29 (SEQ ID NO.: 19) RNase H2 enzyme was added to each reaction. 2,000 copies of synthetic double-stranded gBlock® (IDT, Coralville, IA) template, each corresponding to the homozygous genotypes at the rs113488022 SNP (SEQ ID NOs.: 40 or 41), were added to each reaction. Reactions were performed in triplicate. Reactions were cycled under the following conditions: 95° C.^{3:00}→(95° C.^{0:10}→60° C.^{0:30})×65. Fluorescence data for the intercalated SYBR® Green were collected after each extension time point. After the assay was completed, the data were analyzed, and the average C_{q} and ΔC_{q} values for each pair-wise combination were calculated. The results are presented in Table 11.

TABLE 10
Sequences and SEQ IDs for the primers and
templates used in the experiment described in
Example 4.

| SEQ | | |
|---|---|---|
| ID | | |
| NO.: | Name | Sequence¹ |
| 36 | rs113488022 | GTGATTTTGGTCTAGCTACAGT |
| | For unblocked | |
| 37 | rs113488022 | CCTCAATTCTTACCATCCACAAA |
| | Rev unblocked | |
| 38 | rs113488022 | GTGATTTTGGTCTAGCTACAGTgAAATG-x |
| | TrG 4dmx | |
| 39 | rs113488022 | GTGATTTTGGTCTAGCTACAGAgAAATG-x |
| | ArG 4dmx | |
| 40 | gBlock ® T | TAAGAGGAAAGATGAAGTACTATGTTTTAA |
| | template | AGAATATTATATTACAGAATTATAGAAATT |
| | | AGATCTCTTACCTAAACTCTTCATAATGCTT |
| | | GCTCTGATAGGAAAATGAGATCTACTGTTT |
| | | TCCTTTACTTACTACACCTCAGATATATTTC |
| | | TTCATGAAGACCTCACAGTAAAAATAGGTG |
| | | ATTTTGGTCTAGCTACAGTGAAATCTCGAT |
| | | GGAGTGGGTCCCATCAGTTTGAACAGTTGT |
| | | CTGGATCCATTTTGTGGATGGTAAGAATTG |
| | | AGGCTATTTTTCCACTGATTAAATTTTTGGC |
| | | CCTGAGATGCTGCTGAGTTACTAGAAAGTC |
| | | ATTGAAGGTCTCAACTATAGTATTTTCATA |
| | | GTTCCCAGTATTCACAAAAATCAGTGTTCT |
| | | TATTTTTT |
| 41 | gBlock ® A | TAAGAGGAAAGATGAAGTACTATGTTTTAA |
| | template | AGAATATTATATTACAGAATTATAGAAATT |
| | | AGATCTCTTACCTAAACTCTTCATAATGCTT |
| | | GCTCTGATAGGAAAATGAGATCTACTGTTT |
| | | TCCTTTACTTACTACACCTCAGATATATTTC |
| | | TTCATGAAGACCTCACAGTAAAAATAGGTG |
| | | ATTTTGGTCTAGCTACAGAGAAATCTCGAT |
| | | GGAGTGGGTCCCATCAGTTTGAACAGTTGT |
| | | CTGGATCCATTTTGTGGATGGTAAGAATTG |
| | | AGGCTATTTTTCCACTGATTAAATTTTTGGC |
| | | CCTGAGATGCTGCTGAGTTACTAGAAAGTC |
| | | ATTGAAGGTCTCAACTATAGTATTTTCATA |
| | | GTTCCCAGTATTCACAAAAATCAGTGTTCT |
| | | TATTTTTT |

¹DNA is uppercase, RNA is lowercase. X = C3 spacer (propanediol) blocker group. Location of the mismatch is shown in bold and underlined in the gBlocks ®.

TABLE 11
C_{q} and ΔC_{q} values for the experiments in Example 4.

| RNase | Forward | T | A | | T | A | |
|---|---|---|---|---|---|---|---|
| H2 | Primer | template | template | ΔC_{q} | template | template | ΔC_{q} |
| | | 5 mU RNase H2 | | | 10 mU RNase H2 | | |
| WT | Unblocked | 25.9 | 26.6 | 0.7 | 25.5 | 26.3 | 0.9 |
| | TrG | 27.6 | 31.4 | 3.8 | 26.5 | 30.0 | 3.5 |
| | ArG | 38.1 | 28.0 | 10.1 | 37.4 | 27.4 | 10.0 |
| A107V | Unblocked | 25.6 | 26.2 | 0.6 | 25.4 | 26.1 | 0.8 |
| SEL29 | TrG | 28.0 | 35.9 | 7.8 | 26.9 | 34.2 | 7.3 |
| | ArG | 38.7 | 27.5 | 11.2 | 38.9 | 27.4 | 11.5 |
| WT | Unblocked | 26.0 | 26.6 | 0.6 | 25.4 | 26.2 | 0.7 |
| | TrG | 27.4 | 31.9 | 4.5 | 26.7 | 30.3 | 3.6 |
| | ArG | 38.5 | 27.5 | 10.9 | 37.5 | 27.1 | 10.4 |
| SEL29 | Unblocked | 26.0 | 26.6 | 0.5 | 25.4 | 26.3 | 0.9 |
| | TrG | 29.1 | 36.3 | 7.2 | 26.9 | 34.3 | 7.4 |
| | ArG | 39.6 | 27.8 | 11.8 | 38.4 | 27.5 | 10.9 |
| | | 20 mU RNase H2 | | | 40 mU RNase H2 | | |
| Q48R | Unblocked | 25.7 | 26.5 | 0.9 | 25.4 | 26.2 | 0.8 |
| SEL29 | TrG | 30.1 | 36.2 | 6.1 | 27.8 | 34.7 | 6.9 |
| | ArG | 38.6 | 28.0 | 10.6 | 37.7 | 27.5 | 10.2 |

These data show that Q48R SEL29 and A107V SEL29 RNase H2 significantly improve mismatch discrimination 5′ of the RNA nucleotide. The ΔC_{q} quantification for the TrG primer increases from 3.8 cycles with WT P. a. RNase H2 to 5.9 cycles with A107V SEL29 RNase H2 (for 5 mU of WT RNase H2 and A107V SEL29 RNase H2) and from 4.5 cycles with WT P. a. RNase H2 to 6.9 cycles with Q48R SEL29 RNase H2 (for 5 mU of WT RNase H2 and 40 mU of Q48R SEL29 RNase H2). The ΔC_{q} quantification for the ArG primer increases from 10.1 cycles with WT P. a. RNase H2 to 11.2 cycles with A107V SEL29 RNase H2 (for 5 mU of WT RNase H2 and A107V SEL29 RNase H2), but decreased from 10.9 cycles with WT P. a. RNase H2 to 10.2 cycles with Q48R SEL29 RNase H2 (for 5 mU of WT RNase H2 and 40 mU of Q48R SEL29 RNase H2). The change of the mismatch discrimination with the ArG primer is less crucial, as the ΔC_{q} was already rather effective for this primer using the WT enzyme. These increases in mismatch discrimination for Q48R SEL29 and A107V SEL29 RNase H2 are similar to those for the background SEL29 RNase H2. Q48R SEL29 and A107V SEL29 RNase H2 therefore improve mismatch discrimination when the mismatch is located 5′ of the RNA base.

### Example 5. Q48R SEL29 and A107V SEL29 RNase H2 Increase Mismatch Discrimination Compared to WT RNase H2 When the Mismatch Is Placed 3′ of the RNA

To determine whether Q48R SEL29 and A107V SEL29 RNase H2 enzymes can improve mismatch discrimination when the mismatch is located 3′ of the RNA nucleotide, an assay targeting the rs7583169 and rs3117947 SNPs were designed with the mismatch located immediately 3′ of the RNA. The primers used in this assay are shown in Table 12, SEQ NOs.: 42-47. Assays were run in 10 μL reaction volumes. Thermal cycling and data collection were run on a CFX3848 Real Time System (Bio-Rad®, Hercules, CA). Briefly, either 200 nM (2 pmol) of the blocked forward primer (SEQ ID NOs.: 43 or 46) and 200 nM (2 pmol) of the unblocked reverse primer (SEQ ID NOs.: 44 or 47), or 200 nM (2 pmol) of the unblocked forward primer (SEQ ID NOs.: 42 or 45) and 200 nM (2 pmol) of the unblocked reverse primer (SEQ ID NOs.: 44 or 47) were mixed into 1× of PrimeTime® Gene Expression Master Mix (IDT, Coralville, IA) with 3.0 mM (total) MgCl₂ and 0.5× EvaGreen® Dye (Biotium Inc., Fremont, CA). 5 mU or 10 mU of WT (SEQ ID NO.: 1) P. a. RNase H2 enzyme, 50 mU or 100 mU of Q48R SEL29 (SEQ ID NO.: 18) RNase H2 enzyme, or 20 mU or 40 mU of A107V SEL29 (SEQ ID NO.: 19) RNase H2 enzyme was added to each reaction. 20 ng of genomic cell line DNA (cell lines NA12878 and NA24385, Coriell Institute for Medical Research, Camden, NJ), representing the two homozygous genotypes at the rs7583169 and rs3117947 SNPs were added to each reaction. Reactions were performed in triplicate, and the results averaged. Reactions were cycled under the following conditions: 95° C.^{3:00}→(95° C.^{0:10}→60° C.^{0:30})×65. Fluorescence data for the intercalated EvaGreen® were collected after each extension time point. After the assay was completed, the data were analyzed, and the average C_{q} and ΔC_{q} values for each pair-wise combination were calculated. The results are presented in Table 13.

TABLE 12
Sequences and SEQ IDs for the primers used in the
experiment described in Example 5.

| SEQ | | |
|---|---|---|
| ID | | |
| NO.: | Name | Sequence¹ |
| 42 | rs7583169 For | GGCAGATTTTCTTCTGCACCGCG |
| | unblocked | |
| 43 | rs7583169 For | GGCAGATTTTCTTCTGCACCGCgGT-X- |
| | blocked | TC-X |
| 44 | rs7583169 Rev | TCCCGTCGAGCACCAGCAATTTTACTC |
| | unblocked | |
| 45 | rs3117947 For | CTTTGGATAAGGAAGAAGCCAACT |
| | unblocked | |
| 46 | rs3117947 For | CTTTGGATAAGGAAGAAGCCAACuGC-X- |
| | blocked | AG-X |
| 47 | rs3117947 Rev | GGGAGCTTGAAATGAACAAGGTGAGAAG |
| | unblocked | |

¹DNA is uppercase, RNA is lowercase. X = C3 spacer (propanediol) blocker group. Location of the mismatch is shown in bold and underlined in the gBlocks ®.

TABLE 13
C_{q} and ΔC_{q} values for the experiments in Example 5.

| | | Forward | | | | | | |
|---|---|---|---|---|---|---|---|---|
| | | Primer | | | | | | |
| | | SEQ ID | NA | NA | | NA | NA | |
| RNase H2 | SNP | NO. | 12878 | 24385 | ΔC_{q} | 12878 | 24385 | ΔC_{q} |
| | | | 5 mU RNase H2 | | | 10 mU RNase H2 | | |
| WT | rs7583169 | 42 | 25.3 | 25.1 | −0.2 | 24.8 | 24.6 | −0.2 |
| | | 43 | 26.1 | 26.1 | 0.0 | 25.2 | 25.2 | −0.1 |
| | rs3117947 | 45 | 25.9 | 25.3 | −0.6 | 24.9 | 24.7 | −0.2 |
| | | 46 | 28.0 | 38.7 | 10.7 | 26.2 | 32.6 | 6.3 |
| SEL29 | rs7583169 | 42 | 25.0 | 25.6 | 0.6 | 24.7 | 25.2 | 0.4 |
| (Mut ID 2) | | 43 | 26.0 | 30.5 | 4.5 | 25.5 | 28.2 | 2.8 |
| | rs3117947 | 45 | 25.1 | 24.7 | −0.5 | 25.0 | 25.1 | 0.1 |
| | | 46 | 26.7 | 40.9 | 14.2 | 26.3 | 37.9 | 11.6 |
| | | | 20 mU RNase H2 | | | 40 mU RNase H2 | | |
| A107V | rs7583169 | 42 | 25.0 | 24.9 | −0.2 | 24.6 | 24.8 | 0.2 |
| SEL29 | | 43 | 25.5 | 28.1 | 2.6 | 25.1 | 26.5 | 1.4 |
| (Mut ID 8) | rs3117947 | 45 | 25.3 | 25.2 | −0.1 | 24.7 | 24.9 | 0.1 |
| | | 46 | 32.2 | 52.2 | 20.0 | 28.6 | 50.9 | 22.3 |
| | | | 50 mU RNase H2 | | | 100 mU RNase H2 | | |
| Q48R | rs7583169 | 42 | 25.2 | 24.9 | −0.3 | 25.1 | 24.9 | −0.2 |
| SEL29 | | 43 | 25.9 | 44.5 | 18.6 | 25.3 | 36.8 | 11.5 |
| (Mut ID 7) | rs3117947 | 45 | 25.4 | 25.5 | 0.1 | 25.1 | 24.9 | −0.2 |
| | | 46 | 32.8 | 55.0 | 22.2 | 28.6 | 49.0 | 20.4 |

ΔC_{q} values from Table 13 were calculated as the difference between the C_{q} value for the mismatched template (NA24385) and the C_{q} value for the matched template (NA12878).
The data for SEL29 RNase H2 were collected previously.

These data show that Q48R SEL29 and A107V SEL29 significantly increase mismatch discrimination 3′ of the RNA nucleotide. The ΔC_{q} quantification of the mismatch discrimination for the rs7583169 SNP increases from 0.0 cycles with WT P. a. RNase H2 to 1.4 cycles with A107V SEL29 RNase H2 (for 5 mU of WT RNase H2 and 40 mU of A107V SEL29 RNase H2) and from 0.0 cycles with WT P. a. RNase H2 to 11.5 cycles with Q48R SEL29 RNase H2 (for 5 mU of WT RNase H2 and 100 mU of Q48R SEL29 RNase H2). The ΔC_{q} quantification of the mismatch discrimination for the rs3117947 SNP increases from 10.7 cycles with WT P. a. RNase H2 to 22.3 cycles with A107V SEL29 RNase H2 (for 5 mU of WT RNase H2 and 40 mU of A107V SEL29 RNase H2) and from 10.7 cycles with WT P. a. RNase H2 to 20.4 cycles with Q48R SEL29 RNase H2 (for 5 mU of WT RNase H2 and 100 mU of Q48R SEL29 RNase H2). These increases in mismatch discrimination for Q48R SEL29 and A107V SEL29 RNase H2 are significantly greater than those for the background SEL29 RNase H2. Q48R SEL29 and A107V SEL29 RNase H2 therefore improve mismatch discrimination when the mismatch is located 3′ of the RNA base.

### Example 6. Use of Q48R SEL29 and A107V SEL29 Hybrid RNase H2 Proteins in LAMP Reactions

This example outlines a method of demonstrating the use of hybrid RNase H2 proteins in a LAMP reaction to reduce primer-dimer formation.

To evaluate the functionality of rhPrimers for LAMP protocols that include the different RNase H2 proteins (WT or hybrid RNase H2 proteins), three assays could be designed using (1) unmodified control primers; “Gen1” rDDDDMx primers wherein “r” is an RNA base, “D” is a DNA base, “m” is a mismatch and “x” is a C3 spacer; and “Gen2” rDxxDM primers. The details of the assays to be used in the evaluations are detailed in Tables 14, 15, and 16. LAMP reactions utilizing each type of primer will be held at 25° C. (room temperature) for 0 or 2 hours prior to testing. This will allow for the formation of the primer-dimer products. After the room temperature hold, all reactions will be run at 65° C. for 2 hours in a BioRad CFX384 or Roche LightCycler 480. Signal generation in all of these reactions will be performed with 1× EvaGreen, added to the reaction (see: Biotechnology Letters, December 2007, Volume 29, Issue 12, pp 1939-1946).

TABLE 14
Controlled unmodified primer designs.

| SEQ | | |
|---|---|---|
| ID | | |
| NO.: | Name | Sequence |
| 48 | FIP- | CAGCCAGCCGCAGCACGTTCGCTCATAGGAGATATG |
| | lambda | GTAGAGCCGC |
| 49 | BIP- | GAGAGAATTTGTACCACCTCCCACCGGGCACATAGC |
| | lambda | AGTCCTAGGGACAGT |
| 50 | E3- | GGCTTGGCTCTGCTAACACGTT |
| | lambda | |
| 51 | B3- | GGACGTTTGTAATGTCCGCTCC |
| | lambda | |
| 52 | rhLAMP | F-ACGTGCTGCGgCTGGCTGGT-Q |
| | Probe- | |
| | lambda | |
| 53 | FIP- | CCAAAGAGTAAAGTCCTTCTCTCTCGAGAGACTGTT |
| | hCFTR | GGCCCTTGAAGG |
| 54 | BIP- | GTGTTGATGTTATCCACCTTTTGTGGACTAGGAAAA |
| | hCFTR | CAGATCAATAG |
| 55 | E3-hCFTR | TAATCCTGGAACTCCGGTGC |
| 56 | B3-hCFTR | TTTATGCCAATTAACATTTTGAC |
| 57 | rhLAMP | F-CCTCCCTGTGGATgAGAGAGAAGG-Q |
| | Probe- | |
| | hCFTR | |

DNA bases are uppercase; RNA bases are lowercase. F = 6-carboxyfluorescein; Q = IowaBlack ™ FQ fluorescence quencher.

TABLE 15
Gen1 primer designs.

| SEQ | | |
|---|---|---|
| ID | | |
| NO.: | Name | Sequence |
| 58 | FIP- | CAGCCAGCCGCAGCACGTTCGCTCATAGGAGATATGG |
| | lambda | TAGAGCCGCaGACAG-X |
| 59 | BIP- | GAGAGAATTTGTACCACCTCCCACCGGGCACATAGCA |
| | lambda | GTCCTAGGGACAGTgGCGTT-X |
| 60 | E3- | GGCTTGGCTCTGCTAACACGTTgCTCAA-X |
| | lambda | |
| 61 | B3- | GGACGTTTGTAATGTCCGCTCCgGCACT-X |
| | lambda | |
| 62 | rhLAMP | F-ACGTGCTGCGgCTGGCTGGT-Q |
| | Probe- | |
| | lambda | |
| 63 | FIP- | CCAAAGAGTAAAGTCCTTCTCTCTCGAGAGACTGTTG |
| | hCFTR | GCCCTTGAAGGaGAGCA-X |
| 64 | BIP- | GTGTTGATGTTATCCACCTTTTGTGGACTAGGAAAAC |
| | hCFTR | AGATCAATAGaTAAGC-X |
| 65 | E3- | TAATCCTGGAACTCCGGTGCuAAGGT-X |
| | hCFTR | |
| 66 | B3- | TTTATGCCAATTAACATTTTGACuTTATT-X |
| | hCFTR | |
| 67 | rhLAMP | F-CCTCCCTGTGGATgAGAGAGAAGG-Q |
| | Probe- | |
| | hCFTR | |

DNA bases are uppercase; RNA bases are lowercase. F = 6-carboxyfluorescein; Q = Iowa Black ™ FQ fluorescence quencher. X = C3 spacer.

TABLE 16
GEN2 primer designs.

| SEQ | | |
|---|---|---|
| ID | | |
| NO.: | Name | Sequence |
| 68 | FIP- | CAGCCAGCCGCAGCACGTTCGCTCATAGGAGATATGG |
| | lambda | TAGAGCCGCaG-X-X-AG |
| 69 | BIP- | GAGAGAATTTGTACCACCTCCCACCGGGCACATAGCA |
| | lambda | GTCCTAGGGACAGTgG-X-X-TT |
| 70 | E3- | GGCTTGGCTCTGCTAACACGTTgC-X-X-AA |
| | lambda | |
| 71 | B3- | GGACGTTTGTAATGTCCGCTCCgG-X-X-CT |
| | lambda | |
| 72 | rhLAMP | F-ACGTGCTGCGgCTGGCTGGT-Q |
| | Probe- | |
| | lambda | |
| 73 | FIP- | CCAAAGAGTAAAGTCCTTCTCTCTCGAGAGACTGTTG |
| | hCFTR | GCCCTTGAAGGaG-X-X-CA |
| 74 | BIP- | GTGTTGATGTTATCCACCTTTTGTGGACTAGGAAAAC |
| | hCFTR | AGATCAATAGaT-X-X-GC |
| 75 | E3- | TAATCCTGGAACTCCGGTGCuA-X-X-GT |
| | hCFTR | |
| 76 | B3- | TTTATGCCAATTAACATTTTGACuT-X-X-TT |
| | hCFTR | |
| 77 | rhLAMP | F-CCTCCCTGTGGATgAGAGAGAAGG-Q |
| | Probe- | |
| | hCFTR | |

DNA bases are uppercase; RNA bases are lowercase. F = 6-carboxyfluorescein; Q = Iowa Black ™ FQ fluorescence quencher. X = C3 spacer

Each assay could be performed as follows: The samples will either be Coriell gDNA or Lambda phage genomic DNA. Each assay condition could be run in triplicate with sample input of 5 ng Lambda phage genomic DNA or 20 ng human genomic DNA. For each assay, reactions could run using unmodified primers with intercalating dye (e.g., EvaGreen) and cleavable, blocked primers with an intercalating dye. For each assay, reactions will be run using zero, or titrated levels of RNase H2 (WT or hybrid RNase H2 proteins).

Each assay will be run in triplicate. Comparisons between the unmodified LAMP assays and the modified LAMP assays would be performed, by comparing the length of time required for formation of signal-generating products. It is expected that the hybrid RNase H2 proteins in the LAMP reactions will produce these products significantly later than the reactions containing conventional, wild-type RNase H2 proteins.

The 25 μL EvaGreen reaction mixtures will include:
12.5 μL of 2× Master Mix (1× is 20 mM Tris pH 8.8@25° C., 10 mM KCl, 10 mM (NH₄)₂SO₄, 8 mM MgSO₄, 0.01% Tween-20, 1.4 mM dNTPs.)
1.6 μM FIP primer
1.6 μM BIP primer
1× EvaGreen dye
0.2 μM F3 primer
0.2 μM B3 primer
8 U BST DNA Polymerase (New England Biolabs: https://www.neb.com/products/m0275-bst-dna-polymerase-large-fragment)
1 μL of hybrid RNase H2 protein (for no RNase H2 control, buffer D will be used)
Nuclease Free Water to 25 μL
2 μL sample (either 10 ng/μL human gDNA or 2.5 ng/μL lambda genomic DNA)

### Example 7. Q48R SEL29 RNase H2 Improves the Quality of NGS Libraries Compared to Wild Type P. a. RNase H2 in a Multiplex rhPCR Amplicon Sequencing Workflow Using a 177-Plex Assay Panel

A modified rhAmpSeq protocol was adapted for low frequency variant detection. This protocol uses both a high-fidelity DNA polymerase to reduce amplification errors and unique molecular identifiers (UMIs) for error correction. Q48R SEL29 RNase H2 was compared to wild type P. a. RNase H2 in this system to determine if dimer formation is reduced.

There are two PCR cycling steps with a SPRI (Solid Phase Reversible Immobilization, Beckman Coulter Life sciences, Indianapolis, IN) cleanup following each step. The purpose of the first PCR step (PCR 1) is to incorporate a 6-nucleotide degenerate UMI on each side of the target amplicons. This step also includes the use of 3′-blocked primers and requires RNase H2 to cleave off the blocker and allow a high-fidelity DNA polymerase to extend and amplify each target. The target-specific assay panel contains 177 proprietary primer pairs, and results in approximately 20% of the total reads being primer dimer when wild type P. a. RNase H2 is present in PCR1. his panel generates a large amount of primer dimers, making it optimal for testing Q48R SEL29 RNase H2.

Various 10× titrations of Q48R SEL29 RNase H2 and wild type P. a. RNase H2 (87.5 mU/uL, 175 mU/uL, and 350 mU/uL), were prepared in RNase H2 storage buffer (Integrated DNA technologies, Coralville, IA). Each PCR 1 reaction was 20 μL final volume and contained 10 nM (200 fmol) of each forward and reverse primer, 0.03 U/μL Phusion Hot Start II DNA Polymerase (Thermo Fisher Scientific, Waltham, MA), 20 ng of genomic cell line DNA (cell line NA24385, Coriell Institute for Medical Research, Camden, NJ), and 1× final concentration of RNase H2 in a proprietary high-fidelity polymerase buffer. Thermal cycling was performed on a T100 Thermal Cycler (Bio-Rad®, Hercules, CA) with the cycling conditions listed in Table 17. PCR1 thermal cycling was promptly followed by SPRI cleanup. 1.25× (25 μL) of AMPure magnetic beads (Beckman Coulter Life Sciences, Indianapolis, IN) were added to each well and mixed thoroughly. Plates were incubated at room temperature for 5 minutes on the benchtop followed by 5 minutes on a magnet. The supernatant was discarded and libraries were washed twice with 80% ethanol. Samples were eluted in 22 μL of IDTE pH 7.5 (IDT, Coralville, IA). 20 μL of eluted product was carried forward as input for PCR2.

TABLE 17
PCR 1 cycling conditions.

| | | Temperature | |
|---|---|---|---|
| | Cycles | (° C.) | Time |
| Enzyme | 1 | 95° C. | 10 min |
| activation | | | |
| Amplification | 2 | 95° C. | 15 secs |
| | | 60° C. | 12 min |
| Hold | 1 | 4° C. | ∞ Hold |

The purpose of the second PCR step (PCR 2) was to amplify PCR 1 product and add unique sample index sequences for pooling and sequencing purposes. PCR 2 does not require the use of RNase H2. PCR 2 reactions were performed at 50 μL final volume. A 2× version of the high-fidelity polymerase buffer used in PCR 1 was added to each eluted sample from PCR 1 for a final 1× concentration. Each reaction also had a unique combination of i5 and i7 rhAmpSeq index primers (IDT) at 500 nM each. Thermal cycling was performed on T100 Thermal Cycler (Bio-Rad®, Hercules, CA) with the cycling conditions listed in Table 18. SPRI cleanup immediately followed PCR 2 and is identical as the methods listed above, except for AMPure bead concentrations, which were used at 0.9× after PCR 2. Samples were eluted in 22 uL of IDTE pH 7.5 (IDT, Coralville, IA). 20 μL of eluted product was taken and stored at −20° C. until sequencing.

TABLE 18
PCR 2 cycling Conditions.

| | | Temperature | |
|---|---|---|---|
| | Cycles | (° C.) | Time |
| Enzyme activation | 1 | 95° C. | 30 secs |
| Amplification | 22 | 95° C. | 15 secs |
| | | 60° C. | 30 secs |
| | | 72° C. | 30 secs |
| Final extension | 1 | 72° C. | 1 min |
| Hold | 1 | 4° C. | ∞ Hold |

The resulting libraries were pooled at equal volume for sequencing (5 μL). This pool was quantified using the Qubit dsDNA HS Assay kit (Thermo Fisher), and diluted to 4 nM final concentration. An equal amount of library pool was combined with 0.2 N NaOH to denature libraries. This reaction was subsequently diluted to a final concentration of 8 pM containing 2.5% PhiX (Illumina, San Diego, CA) spike-in. This reaction was loaded onto a MiSeq 300 cycle kit (Illumina, San Diego, CA) and run (Cluster density: 1002±34; Q30: 93.13%). Results were run through the internal proprietary IDT rhAmpSeq VII bioinformatics analysis pipeline.

For the purposes of this disclosure, calculations and terms relating to results below are listed here. The amount of primer dimer produced during amplification of sequencing libraries is defined by the dimer rate. The rhAmpSeq VII pipeline calculates this by taking the overall counts of dimer identified and dividing by the number of QC passed reads (total number of reads that passed a chastity filter). Mapping rate is calculated by taking the total number of mapped reads and dividing by the QC passed reads. On target rate is calculated by taking the total number of on-target reads and dividing by the QC passed reads. Overall amplicon uniformity, or amplicon uniformity≥0.2× is calculated as the percent of normal amplicons that have greater than or equal to 0.2 times the amplicon mean coverage. The dropout rate, or uniformity≤0.05×, is calculated as the percent of normal amplicons with less than 0.05 times the amplicon mean coverage. The amplicon uniformity distribution compares each amplicon's coverage relative to the mean amplicon coverage for a given sample, and plots in the following ranges: 0.1-0.2×, 0.2-0.5×, 0.5-1.5×, 1.5-2.5×, and 2.5-5×.

The rhAmpSeq VII pipeline also identifies the primers contributing to the formation of each primer dimer. The normalized dimer count reflects the percentage that each primer dimer contributes to the total Dimer Rate and is calculated using the following formula:

$$Normalized Dimer Count = \frac{Dimer Weight * Total Dimer Reads per Sample}{Total Reads per Sample}$$

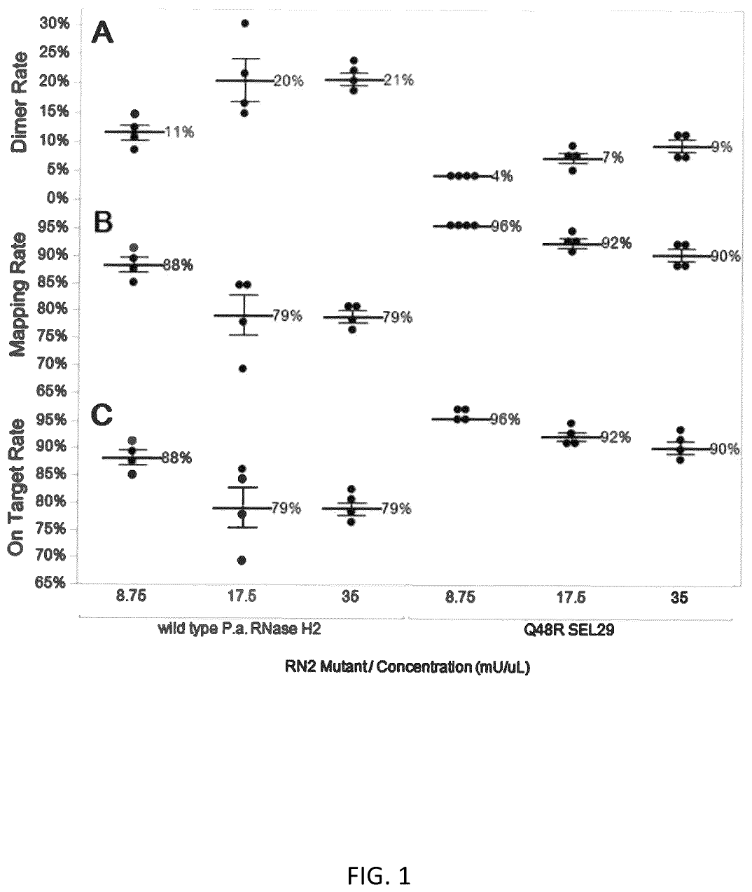

For all concentrations tested, Q48R SEL29 RNase H2 reduced the dimer rate compared to wild type P. a. RNase H2, and also increased mapping rate and on-target rate. At the lowest concentration (8.75 mU/μL) Q48R SEL29 RNase H2 generated libraries with 4% dimer, compared to the control wild type P. a. RNase H2 which generated 11% dimer (FIG. 1, panel A).

The mapping rate and on target rate for libraries produced with the lowest concentration of Q48R SEL29 RNase H2 (8.75 mU/μL) is increased to 96% compared to 88% for wild type P. a. RNase H2 (FIG. 1, panels B and C).

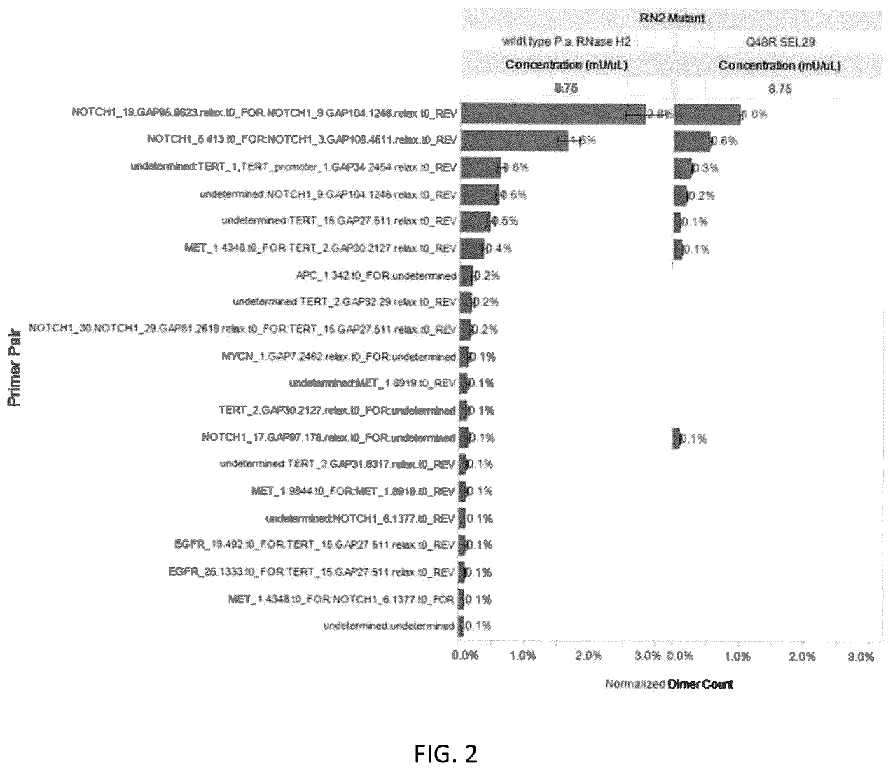

The dimer rate can be broken down into individual primer dimers produced, then normalized to determine each primer dimer's contribution to the overall dimer rate as described above (normalized dimer count). Q48R SEL29 RNase H2 reduces the amount of most primer dimers in half. For example, in libraries generated with the lowest concentration of Q48R Mut 29 RNase H2 (8.75 mU/μL) the top dimer identified in all sample libraries (NOTCH1_19.GAP95.9623.relax.t0_FOR:NOTCH1_ 9.GAP104.1246.relax.t0_REV) only makes up 1.23% of the overall dimer rate compared to wild type P. a. RNase H2 with 2.83%. Some dimers are reduced by more than half in the case of undetermined:TERT_15.GAP27.511.relax.t0_REV Q48R SEL29 RNase H2 libraries generate 75% less dimer at 0.11% compared to wild type P. a. RNase H2 where this dimer contributes 0.47% to the overall Dimer Rate (Table 19 and FIG. 2).

TABLE 19
Normalized Dimer Count per primer pair identified described in
Example 1.

| | Wild type | Q48R |
|---|---|---|
| | P.a. | SEL29 |
| | RNase H2 | RNase H2 |
| | 8.75 mU/ | 8.75 mU/ |
| Primer Pair | μL RN2 | μL RN2 |
| NOTCH1_19.GAP95.9623.relax.t0_FOR: | 2.83% | 1.23% |
| NOTCH1_9.GAP104.1246.relax.t0_REV | | |
| NOTCH1_5.413.t0_FOR: | 1.65% | 0.63% |
| NOTCH1_3.GAP109.4611.relax.t0_REV | | |
| undetermined: | 0.63% | 0.32% |
| TERT_1, | | |
| TERT_promoter_1.GAP34.2454.relax.t0_REV | | |
| undetermined: | 0.47% | 0.11% |
| TERT_15.GAP27.511.relax.t0_REV | | |
| undetermined: | 0.61% | 0.29% |
| NOTCH1_9.GAP104.1246.relax.t0_REV | | |
| MET_1.4348.t0_FOR: | 0.38% | 0.16% |
| TERT_2.GAP30.2127.relax.t0_REV | | |
| APC_1.342.t0_FOR: | 0.21% | not |
| undetermined | | detected |
| undetermined: | 0.20% | 0.07% |
| TERT_2.GAP32.29.relax.t0_REV | | |
| NOTCH1_30, | 0.18% | 0.03% |
| NOTCH1_29.GAP81.2618.relax.t0_FOR | | |
| TERT_15.GAP27.511.relax.t0_REV | | |
| undetermined: | 0.12% | 0.06% |
| MET_1.8919.t0_REV | | |
| TERT_2.GAP30.2127.relax.t0_FOR: | 0.13% | 0.06% |
| undetermined | | |
| MYCN_1.GAP7.2462.relax.t0_FOR: | 0.13% | 0.05% |
| undetermined | | |
| EGFR_19.492.t0_FOR: | 0.10% | not |
| TERT_15.GAP27.511.relax.t0_REV | | detected |
| EGFR_25.1333.t0_FOR: | 0.10% | not |
| TERT_15.GAP27.511.relax.t0_REV | | detected |
| NOTCH1_17.GAP97.178.relax.t0_FOR: | 0.13% | 0.15% |
| undetermined | | |
| TERT_7.69.t0_FOR: | 0.07% | 0.07% |
| MYC_2.884.t0_REV | | |
| NOTCH1_34.GAP74.1722.relax.t0_FOR: | 0.07% | 0.03% |
| undetermined | | |
| MET_1.9844.t0_FOR: | 0.12% | 0.08% |
| MET_1.8919.t0_REV | | |
| MET_1.4348.t0_FOR: | 0.09% | 0.04% |
| NOTCH1_6.1377.t0_FOR | | |
| TERT_7.69.t0_FOR: | not | 0.06% |
| MYCL_2.GAP2.2394.relax.t0_REV | detected | |
| undetermined: | not | 0.03% |
| NOTCH1_12,NOTCH1_11.207.t0_REV | detected | |
| NOTCH1_19.GAP95.9623.relax.t0_FOR: | 0.06% | 0.05% |
| NOTCH1_2.45.t0_REV | | |
| DNMT3B_15.337.t0_REV: | not | 0.06% |
| DNMT3B_15.337.t0_REV | detected | |
| MAP3K1_1.GAP39.5178.relax.t0_FOR: | not | 0.06% |
| TERT_2.GAP33.6839.relax.t0_REV | detected | |
| TERT_1, | not | 0.05% |
| TERT_promoter_1.GAP34.14490.relax.t0_FOR: | detected | |
| undetermined | | |
| TERT_5.GAP29.1338.relax.t0_FOR: | not | 0.07% |
| undetermined | detected | |
| undetermined: | 0.09% | 0.10% |
| NOTCH1_6.1377.t0_REV | | |
| undetermined: | not | 0.06% |
| NOTCH1_8.381.t0_REV | detected | |
| undetermined: | not | 0.09% |
| NOTCH1_22.GAP94.2342.relax.t0_REV | detected | |
| undetermined: | 0.10% | 0.13% |
| TERT_2.GAP31.8317.relax.t0_REV | | |
| undetermined: | 0.10% | 0.04% |
| undetermined | | |

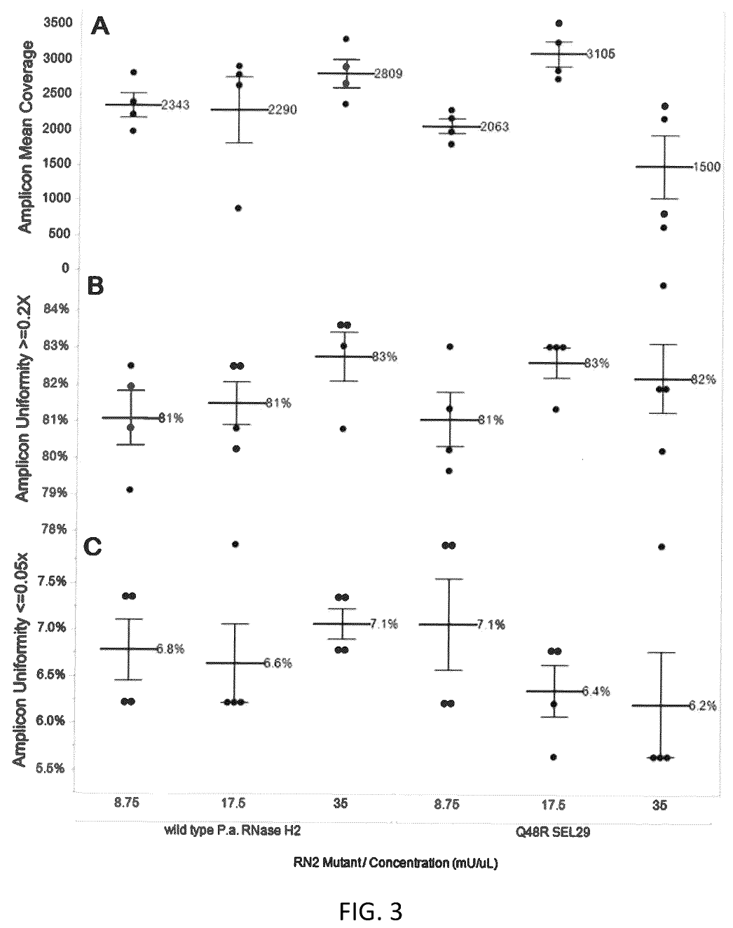

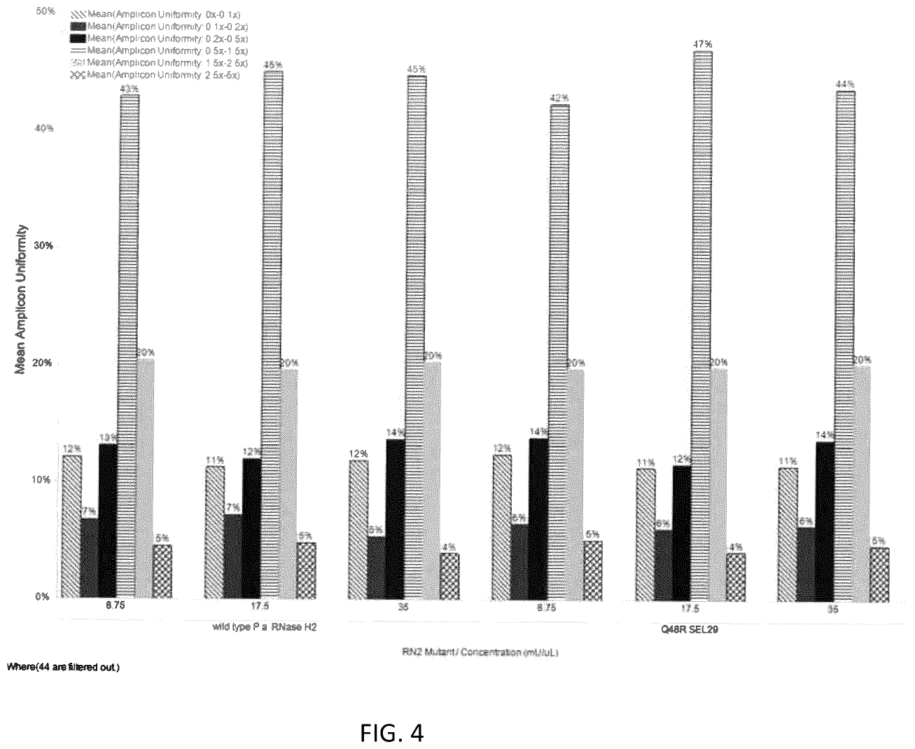

The ability of Q48R SEL29 RNase H2 to reduce the dimer rate does not impact the library yield, overall uniformity and dropout rates, or uniformity distribution metrics. Q48R SEL29 RNase H2 generates similar library yields with all titration concentrations tested compared to the wild type P. a. RNase H2 enzymes as demonstrated by the amplicon mean coverage (FIG. 3, panel A). In addition, the overall amplicon uniformity≥0.2× and amplicon dropout rates (amplicon uniformity≤0.05×) are comparable in all titration concentrations tested between the Q48R SEL29 RNase H2 and the wild type P. a. enzyme (FIG. 3, panels B and C). Further, the uniformity distribution between Q48R SEL29 and wild type P. a. RNase H2 appears similar (FIG. 4). Q48R SEL29 RNase H2 reduces primer dimer formation compared to standard wild-type P. a. RNase H2 during library generation in a rhAmpseq workflow without altering other important sequencing metrics.

Together, these data show that Q48R SEL29 RNase H2 improves generation of multiplex next generation sequencing libraries using a high-fidelity DNA polymerase in a high-fidelity buffer.

### Example 8. Exemplary Amino Acid and Nucleic Acid Sequences Encoding RNase H2 Proteins

Exemplary amino acid and nucleic acid sequences encoding RNase H2 proteins are presented below.

TABLE 20
Nucleic acid sequences encoding RNase H2 proteins
previously described.

| Mut | SEQ | Specific | |
|---|---|---|---|
| ID | ID | AA | |
| # | NO.: | changes | Sequence¹ |
| N/A | 78 | WT P.a. | ATGAAAGTTGCAGGTGCAGATGAAGCTGGTC |
| | | RNase H2 | GTGGTCCAGTTATTGGTCCGCTGGTTATTGTT |
| | | | GCTGCTGTTGTGGAGGAAGACAAAATCCGCT |
| | | | CTCTGACTAAGCTGGGTGTTAAAGACTCCAAA |
| | | | CAGCTGACCCCGGCGCAACGTGAAAAACTGT |
| | | | TCGATGAAATCGTAAAAGTACTGGATGATTAC |
| | | | TCTGTGGTCATTGTGTCCCCGCAGGACATTGA |
| | | | CGGTCGTAAGGGCAGCATGAACGAACTGGAG |
| | | | GTAGAAAACTTCGTTAAAGCCCTGAATAGCCT |
| | | | GAAAGTTAAGCCGGAAGTTATTTACATTGATT |
| | | | CCGCTGATGTTAAAGCTGAACGTTTCGCTGAA |
| | | | AACATTCGCAGCCGTCTGGCGTACGAAGCGA |
| | | | AAGTTGTAGCCGAACATAAAGCGGATGCGAA |
| | | | GTATGAGATCGTATCCGCAGCCTCTATCCTGG |
| | | | CAAAAGTTATCCGTGACCGCGAGATCGAAAA |
| | | | GCTGAAAGCCGAATACGGTGATTTTGGTTCCG |
| | | | GTTACCCGTCTGATCCGCGTACTAAGAAATGG |
| | | | CTGGAAGAATGGTATAGCAAACACGGCAATT |
| | | | TCCCGCCGATCGTGCGTCGTACTTGGGATACT |
| | | | GCAAAGAAAATCGAAGAAAAATTCAAACGTG |
| | | | CGCAGCTGACCCTGGACAACTTCCTGAAGCGT |
| | | | TTTCGCAACAAGCTTGCGGCCGCACTCGAGATC |
| | | | AAACGGGCTAGCCAGCCAGAACTCGCCCCGGAA |
| | | | GACCCCGAGGATGTCGAGCACCACCACCACCAC |
| | | | CACTGA |
| 1 | 79 | SEL28 | ATGAAAGTTGCAGGTGCAGATGAAGCTGGTC |
| | | RNase H2 | GTGGTCCAGTTATTGGTCCGCTGGTTATTGTT |
| | | | GCTGCTGTTGTGGATGAAAACAGTCTCCCCAA |
| | | | GCTGGAAGAGCTGAAAGTTAGAGACTCCAAA |
| | | | AAGCTGACCCCGAAGCGACGTGAAAAACTGT |
| | | | TCGATGAAATCGTAAAAGTACTGGATGATTAC |
| | | | TCTGTGGTCATTGTGTCCCCGCAGGACATTGA |
| | | | CGGTCGTAAGGGCAGCATGAACGAACTGGAG |
| | | | GTAGAAAACTTCGTTAAAGCCCTGAATAGCCT |
| | | | GAAAGTTAAGCCGGATGTTATTTACGCTGATG |
| | | | CCGCTGATGTTGATGAAGAACGTTTCGCTAGA |
| | | | GAGCTTGGCGAGCGTCTGAACTTCGAAGCGG |
| | | | AAGTTGTAGCCAAACATAAAGCGGATGACAT |
| | | | CTTTCCGGTCGTATCCGCAGCCTCTATCCTGG |
| | | | CAAAAGTTATCCGTGACCGCGAGATCGAAAA |
| | | | GCTGAAAGCCGAATACGGTGATTTTGGTTCCG |
| | | | GTTACCCGTCTGATCCGCGTACTAAGAAATGG |
| | | | CTGGAAGAATGGTATAGCAAACACGGCAATT |
| | | | TCCCGCCGATCGTGCGTCGTACTTGGGATACT |
| | | | GCAAAGAAAATCGAAGAAAAATTCAAACGTG |
| | | | CGCAGCTGACCCTGGACAAGTTCTTCAAGAA |
| | | | ACCTAAGCTTGCGGCCGCACTCGAGATCAAACG |
| | | | GGCTAGCCAGCCAGAACTCGCCCCGGAAGACCC |
| | | | CGAGGATGTCGAGCACCACCACCACCACCACTG |
| | | | A |
| 2 | 80 | SEL29 | ATGAAAGTTGCAGGTATAGATGAAGCTGGTC |
| | | RNase H2 | GTGGTCCAGCTATTGGTCCGCTGGTTATTGTT |
| | | | GCTGCTGTTGTGGATGAAAACAGTCTCCCCAA |
| | | | GCTGGAAGAGCTGAAAGTTAGAGACTCCAAA |
| | | | AAGCTGACCCCGGCGCAACGTGAAAAACTGT |
| | | | TCGATGAAATCGTAAAAGTACTGGATGATTAC |
| | | | TCTGTGGTCATTGTGTCCCCGCAGGACATTGA |
| | | | CGGTCGTAAGGGCAGCATGAACGAACTGGAG |
| | | | GTAGAAAACTTCGTTAAAGCCCTGAATAGCCT |
| | | | GAAAGTTAAGCCGGATGTTATTTACGCTGATG |
| | | | CCGCTGATGTTGATGAAGAACGTTTCGCTAGA |
| | | | GAGCTTGGCGAGCGTCTGAACTTCGAAGCGA |
| | | | AAGTTGTAGCCGAACATAAAGCGGATGCGAA |
| | | | GTATGAGATCGTATCCGCAGCCTCTATCCTGG |
| | | | CAAAAGTTATCCGTGACCGCGAGATCGAAAA |
| | | | GCTGAAAGCCGAATACGGTGATTTTGGTTCCG |
| | | | GTTACCCGTCTGATCCGCGTACTAGGGCATTC |
| | | | CTGGAAAATTACTATAGAGAACACGGCGAAT |
| | | | TCCCGCCGATCGTGCGTAAAGGTTGGAAGACT |
| | | | CTAAAGAAAATCGCAGAAAAAGTCGAAAGTG |
| | | | AGAAGAAGGCTGAGGAACGGCAGGCGACCCT |
| | | | GGACAGGTACTTCAGGAAGGTTAAGCTTGCGG |
| | | | CCGCACTCGAGATCAAACGGGCTAGCCAGCCAG |
| | | | AACTCGCCCCGGAAGACCCCGAGGATGTCGAGC |
| | | | ACCACCACCACCACCACTGA |
| 3 | 81 | P13S | ATGAAAGTTGCAGGTGCAGATGAAGCTGGTC |
| | | SEL28 | GTGGTTCTGTTATTGGTCCGCTGGTTATTGTT |
| | | RNase H2 | GCTGCTGTTGTGGATGAAAACAGTCTCCCCAA |
| | | | GCTGGAAGAGCTGAAAGTTAGAGACTCCAAA |
| | | | AAGCTGACCCCGAAGCGACGTGAAAAACTGT |
| | | | TCGATGAAATCGTAAAAGTACTGGATGATTAC |
| | | | TCTGTGGTCATTGTGTCCCCGCAGGACATTGA |
| | | | CGGTCGTAAGGGCAGCATGAACGAACTGGAG |
| | | | GTAGAAAACTTCGTTAAAGCCCTGAATAGCCT |
| | | | GAAAGTTAAGCCGGATGTTATTTACGCTGATG |
| | | | CCGCTGATGTTGATGAAGAACGTTTCGCTAGA |
| | | | GAGCTTGGCGAGCGTCTGAACTTCGAAGCGG |
| | | | AAGTTGTAGCCAAACATAAAGCGGATGACAT |
| | | | CTTTCCGGTCGTATCCGCAGCCTCTATCCTGG |
| | | | CAAAAGTTATCCGTGACCGCGAGATCGAAAA |
| | | | GCTGAAAGCCGAATACGGTGATTTTGGTTCCG |
| | | | GTTACCCGTCTGATCCGCGTACTAAGAAATGG |
| | | | CTGGAAGAATGGTATAGCAAACACGGCAATT |
| | | | TCCCGCCGATCGTGCGTCGTACTTGGGATACT |
| | | | GCAAAGAAAATCGAAGAAAAATTCAAACGTG |
| | | | CGCAGCTGACCCTGGACAAGTTCTTCAAGAA |
| | | | ACCTAAGCTTGCGGCCGCACTCGAGATCAAACG |
| | | | GGCTAGCCAGCCAGAACTCGCCCCGGAAGACCC |
| | | | CGAGGATGTCGAGCACCACCACCACCACCACTG |
| | | | A |
| 4 | 82 | A107V | ATGAAAGTTGCAGGTGCAGATGAAGCTGGTC |
| | | SEL28 | GTGGTCCAGTTATTGGTCCGCTGGTTATTGTT |
| | | RNase H2 | GCTGCTGTTGTGGATGAAAACAGTCTCCCCAA |
| | | | GCTGGAAGAGCTGAAAGTTAGAGACTCCAAA |
| | | | AAGCTGACCCCGAAGCGACGTGAAAAACTGT |
| | | | TCGATGAAATCGTAAAAGTACTGGATGATTAC |
| | | | TCTGTGGTCATTGTGTCCCCGCAGGACATTGA |
| | | | CGGTCGTAAGGGCAGCATGAACGAACTGGAG |
| | | | GTAGAAAACTTCGTTAAAGCCCTGAATAGCCT |
| | | | GAAAGTTAAGCCGGATGTTATTTACGTAGAT |
| | | | GCCGCTGATGTTGATGAAGAACGTTTCGCTAG |
| | | | AGAGCTTGGCGAGCGTCTGAACTTCGAAGCG |
| | | | GAAGTTGTAGCCAAACATAAAGCGGATGACA |
| | | | TCTTTCCGGTCGTATCCGCAGCCTCTATCCTG |
| | | | GCAAAAGTTATCCGTGACCGCGAGATCGAAA |
| | | | AGCTGAAAGCCGAATACGGTGATTTTGGTTCC |
| | | | GGTTACCCGTCTGATCCGCGTACTAAGAAATG |
| | | | GCTGGAAGAATGGTATAGCAAACACGGCAAT |
| | | | TTCCCGCCGATCGTGCGTCGTACTTGGGATAC |
| | | | TGCAAAGAAAATCGAAGAAAAATTCAAACGT |
| | | | GCGCAGCTGACCCTGGACAAGTTCTTCAAGA |
| | | | AACCTAAGCTTGCGGCCGCACTCGAGATCAAAC |
| | | | GGGCTAGCCAGCCAGAACTCGCCCCGGAAGACC |
| | | | CCGAGGATGTCGAGCACCACCACCACCACCACT |
| | | | GA |
| 5 | 83 | P13S/ | ATGAAAGTTGCAGGTGCAGATGAAGCTGGTC |
| | | A107V | GTGGTTCTGTTATTGGTCCGCTGGTTATTGTT |
| | | SEL28 | GCTGCTGTTGTGGATGAAAACAGTCTCCCCAA |
| | | RNase H2 | GCTGGAAGAGCTGAAAGTTAGAGACTCCAAA |
| | | | AAGCTGACCCCGAAGCGACGTGAAAAACTGT |
| | | | TCGATGAAATCGTAAAAGTACTGGATGATTAC |
| | | | TCTGTGGTCATTGTGTCCCCGCAGGACATTGA |
| | | | CGGTCGTAAGGGCAGCATGAACGAACTGGAG |
| | | | GTAGAAAACTTCGTTAAAGCCCTGAATAGCCT |
| | | | GAAAGTTAAGCCGGATGTTATTTACGTAGAT |
| | | | GCCGCTGATGTTGATGAAGAACGTTTCGCTAG |
| | | | AGAGCTTGGCGAGCGTCTGAACTTCGAAGCG |
| | | | GAAGTTGTAGCCAAACATAAAGCGGATGACA |
| | | | TCTTTCCGGTCGTATCCGCAGCCTCTATCCTG |
| | | | GCAAAAGTTATCCGTGACCGCGAGATCGAAA |
| | | | AGCTGAAAGCCGAATACGGTGATTTTGGTTCC |
| | | | GGTTACCCGTCTGATCCGCGTACTAAGAAATG |
| | | | GCTGGAAGAATGGTATAGCAAACACGGCAAT |
| | | | TTCCCGCCGATCGTGCGTCGTACTTGGGATAC |
| | | | TGCAAAGAAAATCGAAGAAAAATTCAAACGT |
| | | | GCGCAGCTGACCCTGGACAAGTTCTTCAAGA |
| | | | AACCTAAGCTTGCGGCCGCACTCGAGATCAAAC |
| | | | GGGCTAGCCAGCCAGAACTCGCCCCGGAAGACC |
| | | | CCGAGGATGTCGAGCACCACCACCACCACCACT |
| | | | GA |
| 6 | 84 | P13S | ATGAAAGTTGCAGGTATAGATGAAGCTGGTC |
| | | SEL29 | GTGGTTCTGCTATTGGTCCGCTGGTTATTGTT |
| | | RNase H2 | GCTGCTGTTGTGGATGAAAACAGTCTCCCCAA |
| | | | GCTGGAAGAGCTGAAAGTTAGAGACTCCAAA |
| | | | AAGCTGACCCCGGCGCAACGTGAAAAACTGT |
| | | | TCGATGAAATCGTAAAAGTACTGGATGATTAC |
| | | | TCTGTGGTCATTGTGTCCCCGCAGGACATTGA |
| | | | CGGTCGTAAGGGCAGCATGAACGAACTGGAG |
| | | | GTAGAAAACTTCGTTAAAGCCCTGAATAGCCT |
| | | | GAAAGTTAAGCCGGATGTTATTTACGCTGATG |
| | | | CCGCTGATGTTGATGAAGAACGTTTCGCTAGA |
| | | | GAGCTTGGCGAGCGTCTGAACTTCGAAGCGA |
| | | | AAGTTGTAGCCGAACATAAAGCGGATGCGAA |
| | | | GTATGAGATCGTATCCGCAGCCTCTATCCTGG |
| | | | CAAAAGTTATCCGTGACCGCGAGATCGAAAA |
| | | | GCTGAAAGCCGAATACGGTGATTTTGGTTCCG |
| | | | GTTACCCGTCTGATCCGCGTACTAGGGCATTC |
| | | | CTGGAAAATTACTATAGAGAACACGGCGAAT |
| | | | TCCCGCCGATCGTGCGTAAAGGTTGGAAGACT |
| | | | CTAAAGAAAATCGCAGAAAAAGTCGAAAGTG |
| | | | AGAAGAAGGCTGAGGAACGGCAGGCGACCCT |
| | | | GGACAGGTACTTCAGGAAGGTTAAGCTTGCGG |
| | | | CCGCACTCGAGATCAAACGGGCTAGCCAGCCAG |
| | | | AACTCGCCCCGGAAGACCCCGAGGATGTCGAGC |
| | | | ACCACCACCACCACCACTGA |
| 7 | 85 | Q48R | ATGAAAGTTGCAGGTATAGATGAAGCTGGTC |
| | | SEL29 | GTGGTCCAGCTATTGGTCCGCTGGTTATTGTT |
| | | RNase H2 | GCTGCTGTTGTGGATGAAAACAGTCTCCCCAA |
| | | | GCTGGAAGAGCTGAAAGTTAGAGACTCCAAA |
| | | | AAGCTGACCCCGGCGCGCCGTGAAAAACTGT |
| | | | TCGATGAAATCGTAAAAGTACTGGATGATTAC |
| | | | TCTGTGGTCATTGTGTCCCCGCAGGACATTGA |
| | | | CGGTCGTAAGGGCAGCATGAACGAACTGGAG |
| | | | GTAGAAAACTTCGTTAAAGCCCTGAATAGCCT |
| | | | GAAAGTTAAGCCGGATGTTATTTACGCTGATG |
| | | | CCGCTGATGTTGATGAAGAACGTTTCGCTAGA |
| | | | GAGCTTGGCGAGCGTCTGAACTTCGAAGCGA |
| | | | AAGTTGTAGCCGAACATAAAGCGGATGCGAA |
| | | | GTATGAGATCGTATCCGCAGCCTCTATCCTGG |
| | | | CAAAAGTTATCCGTGACCGCGAGATCGAAAA |
| | | | GCTGAAAGCCGAATACGGTGATTTTGGTTCCG |
| | | | GTTACCCGTCTGATCCGCGTACTAGGGCATTC |
| | | | CTGGAAAATTACTATAGAGAACACGGCGAAT |
| | | | TCCCGCCGATCGTGCGTAAAGGTTGGAAGACT |
| | | | CTAAAGAAAATCGCAGAAAAAGTCGAAAGTG |
| | | | AGAAGAAGGCTGAGGAACGGCAGGCGACCCT |
| | | | GGACAGGTACTTCAGGAAGGTTAAGCTTGCGG |
| | | | CCGCACTCGAGATCAAACGGGCTAGCCAGCCAG |
| | | | AACTCGCCCCGGAAGACCCCGAGGATGTCGAGC |
| | | | ACCACCACCACCACCACTGA |
| 8 | 86 | A107V | ATGAAAGTTGCAGGTATAGATGAAGCTGGTC |
| | | SEL29 | GTGGTCCAGCTATTGGTCCGCTGGTTATTGTT |
| | | RNase H2 | GCTGCTGTTGTGGATGAAAACAGTCTCCCCAA |
| | | | GCTGGAAGAGCTGAAAGTTAGAGACTCCAAA |
| | | | AAGCTGACCCCGGCGCAACGTGAAAAACTGT |
| | | | TCGATGAAATCGTAAAAGTACTGGATGATTAC |
| | | | TCTGTGGTCATTGTGTCCCCGCAGGACATTGA |
| | | | CGGTCGTAAGGGCAGCATGAACGAACTGGAG |
| | | | GTAGAAAACTTCGTTAAAGCCCTGAATAGCCT |
| | | | GAAAGTTAAGCCGGATGTTATTTACGCTGATG |
| | | | CCGTAGATGTTGATGAAGAACGTTTCGCTAG |
| | | | AGAGCTTGGCGAGCGTCTGAACTTCGAAGCG |
| | | | AAAGTTGTAGCCGAACATAAAGCGGATGCGA |
| | | | AGTATGAGATCGTATCCGCAGCCTCTATCCTG |
| | | | GCAAAAGTTATCCGTGACCGCGAGATCGAAA |
| | | | AGCTGAAAGCCGAATACGGTGATTTTGGTTCC |
| | | | GGTTACCCGTCTGATCCGCGTACTAGGGCATT |
| | | | CCTGGAAAATTACTATAGAGAACACGGCGAA |
| | | | TTCCCGCCGATCGTGCGTAAAGGTTGGAAGAC |
| | | | TCTAAAGAAAATCGCAGAAAAAGTCGAAAGT |
| | | | GAGAAGAAGGCTGAGGAACGGCAGGCGACCC |
| | | | TGGACAGGTACTTCAGGAAGGTTAAGCTTGCG |
| | | | GCCGCACTCGAGATCAAACGGGCTAGCCAGCCA |
| | | | GAACTCGCCCCGGAAGACCCCGAGGATGTCGAG |
| | | | CACCACCACCACCACCACTGA |
| 9 | 87 | P13S/ | ATGAAAGTTGCAGGTATAGATGAAGCTGGTC |
| | | A107V | GTGGTTCTGCTATTGGTCCGCTGGTTATTGTT |
| | | SEL29 | GCTGCTGTTGTGGATGAAAACAGTCTCCCCAA |
| | | RNase H2 | GCTGGAAGAGCTGAAAGTTAGAGACTCCAAA |
| | | | AAGCTGACCCCGGCGCAACGTGAAAAACTGT |
| | | | TCGATGAAATCGTAAAAGTACTGGATGATTAC |
| | | | TCTGTGGTCATTGTGTCCCCGCAGGACATTGA |
| | | | CGGTCGTAAGGGCAGCATGAACGAACTGGAG |
| | | | GTAGAAAACTTCGTTAAAGCCCTGAATAGCCT |
| | | | GAAAGTTAAGCCGGATGTTATTTACGCTGATG |
| | | | CCGTAGATGTTGATGAAGAACGTTTCGCTAG |
| | | | AGAGCTTGGCGAGCGTCTGAACTTCGAAGCG |
| | | | AAAGTTGTAGCCGAACATAAAGCGGATGCGA |
| | | | AGTATGAGATCGTATCCGCAGCCTCTATCCTG |
| | | | GCAAAAGTTATCCGTGACCGCGAGATCGAAA |
| | | | AGCTGAAAGCCGAATACGGTGATTTTGGTTCC |
| | | | GGTTACCCGTCTGATCCGCGTACTAGGGCATT |
| | | | CCTGGAAAATTACTATAGAGAACACGGCGAA |
| | | | TTCCCGCCGATCGTGCGTAAAGGTTGGAAGAC |
| | | | TCTAAAGAAAATCGCAGAAAAAGTCGAAAGT |
| | | | GAGAAGAAGGCTGAGGAACGGCAGGCGACCC |
| | | | TGGACAGGTACTTCAGGAAGGTTAAGCTTGCG |
| | | | GCCGCACTCGAGATCAAACGGGCTAGCCAGCCA |
| | | | GAACTCGCCCCGGAAGACCCCGAGGATGTCGAG |
| | | | CACCACCACCACCACCACTGA |

¹Location of mutations are shown in bold and underlined. Plasmid extension and (His)₆-tag is shown in italics, including the stop codon.

TABLE 21
Amino acid sequences for the RNase H2 proteins
lacking C-terminal extension sequences.

| SEQ | | |
|---|---|---|
| ID | Specific AA | |
| NO.: | changes | Sequence¹ |
| 88 | P13S SEL28 | MKVAGADEAGRGSVIGPLVIVAAVVDENS |
| | RNase H2 | LPKLEELKVRDSKKLTPKRREKLFDEIVKV |
| | | LDDYSVVIVSPQDIDGRKGSMNELEVENFV |
| | | KALNSLKVKPDVIYADAADVDEERFAREL |
| | | GERLNFEAEVVAKHKADDIFPVVSAASILA |
| | | KVIRDREIEKLKAEYGDFGSGYPSDPRTKK |
| | | WLEEWYSKHGNFPPIVRRTWDTAKKIEEK |
| | | FKRAQLTLDKFFKKP |
| 89 | A107V | MKVAGADEAGRGPVIGPLVIVAAVVDENS |
| | SEL28 RNase | LPKLEELKVRDSKKLTPKRREKLFDEIVKV |
| | H2 | LDDYSVVIVSPQDIDGRKGSMNELEVENFV |
| | | KALNSLKVKPDVIYADAVDVDEERFAREL |
| | | GERLNFEAEVVAKHKADDIFPVVSAASILA |
| | | KVIRDREIEKLKAEYGDFGSGYPSDPRTKK |
| | | WLEEWYSKHGNFPPIVRRTWDTAKKIEEK |
| | | FKRAQLTLDKFFKKP |
| 90 | P13S/A107V | MKVAGADEAGRGSVIGPLVIVAAVVDENS |
| | SEL28 RNase | LPKLEELKVRDSKKLTPKRREKLFDEIVKV |
| | H2 | LDDYSVVIVSPQDIDGRKGSMNELEVENFV |
| | | KALNSLKVKPDVIYADAVDVDEERFAREL |
| | | GERLNFEAEVVAKHKADDIFPVVSAASILA |
| | | KVIRDREIEKLKAEYGDFGSGYPSDPRTKK |
| | | WLEEWYSKHGNFPPIVRRTWDTAKKIEEK |
| | | FKRAQLTLDKFFKKP |
| 91 | P13S SEL29 | MKVAGIDEAGRGSAIGPLVIVAAVVDENSL |
| | RNase H2 | PKLEELKVRDSKKLTPAQREKLFDEIVKVL |
| | | DDYSVVIVSPQDIDGRKGSMNELEVENFVK |
| | | ALNSLKVKPDVIYADAADVDEERFARELG |
| | | ERLNFEAKVVAEHKADAKYEIVSAASILAK |
| | | VIRDREIEKLKAEYGDFGSGYPSDPRTRAFL |
| | | ENYYREHGEFPPIVRKGWKTLKKIAEKVES |
| | | EKKAEERQATLDRYFRKV |
| 92 | Q48R SEL29 | MKVAGIDEAGRGPAIGPLVIVAAVVDENSL |
| | RNase H2 | PKLEELKVRDSKKLTPARREKLFDEIVKVL |
| | | DDYSVVIVSPQDIDGRKGSMNELEVENFVK |
| | | ALNSLKVKPDVIYADAADVDEERFARELG |
| | | ERLNFEAKVVAEHKADAKYEIVSAASILAK |
| | | VIRDREIEKLKAEYGDFGSGYPSDPRTRAFL |
| | | ENYYREHGEFPPIVRKGWKTLKKIAEKVES |
| | | EKKAEERQATLDRYFRKV |
| 93 | A107V | MKVAGIDEAGRGPAIGPLVIVAAVVDENSL |
| | SEL29 RNase | PKLEELKVRDSKKLTPAQREKLFDEIVKVL |
| | H2 | DDYSVVIVSPQDIDGRKGSMNELEVENFVK |
| | | ALNSLKVKPDVIYADAVDVDEERFARELG |
| | | ERLNFEAKVVAEHKADAKYEIVSAASILAK |
| | | VIRDREIEKLKAEYGDFGSGYPSDPRTRAFL |
| | | ENYYREHGEFPPIVRKGWKTLKKIAEKVES |
| | | EKKAEERQATLDRYFRKV |
| 94 | P13S/A107V | MKVAGIDEAGRGSAIGPLVIVAAVVDENSL |
| | SEL29 RNase | PKLEELKVRDSKKLTPAQREKLFDEIVKVL |
| | H2 | DDYSVVIVSPQDIDGRKGSMNELEVENFVK |
| | | ALNSLKVKPDVIYADAVDVDEERFARELG |
| | | ERLNFEAKVVAEHKADAKYEIVSAASILAK |
| | | VIRDREIEKLKAEYGDFGSGYPSDPRTRAFL |
| | | ENYYREHGEFPPIVRKGWKTLKKIAEKVES |
| | | EKKAEERQATLDRYFRKV |

¹Location of mutations are shown in bold and underlined.

TABLE 22
Nucleic acid sequences encoding RNase H2 proteins
lacking additional C-terminal extension sequences.

| SEQ | | |
|---|---|---|
| ID | Specific AA | |
| NO.: | changes | Sequence¹ |
| 95 | P13S | ATGAAAGTTGCAGGTGCAGATGAAGCTGGTC |
| | SEL28 | GTGGTTCTGTTATTGGTCCGCTGGTTATTGTT |
| | RNase H2 | GCTGCTGTTGTGGATGAAAACAGTCTCCCCAA |
| | | GCTGGAAGAGCTGAAAGTTAGAGACTCCAAA |
| | | AAGCTGACCCCGAAGCGACGTGAAAAACTGT |
| | | TCGATGAAATCGTAAAAGTACTGGATGATTAC |
| | | TCTGTGGTCATTGTGTCCCCGCAGGACATTGA |
| | | CGGTCGTAAGGGCAGCATGAACGAACTGGAG |
| | | GTAGAAAACTTCGTTAAAGCCCTGAATAGCCT |
| | | GAAAGTTAAGCCGGATGTTATTTACGCTGATG |
| | | CCGCTGATGTTGATGAAGAACGTTTCGCTAGA |
| | | GAGCTTGGCGAGCGTCTGAACTTCGAAGCGG |
| | | AAGTTGTAGCCAAACATAAAGCGGATGACAT |
| | | CTTTCCGGTCGTATCCGCAGCCTCTATCCTGG |
| | | CAAAAGTTATCCGTGACCGCGAGATCGAAAA |
| | | GCTGAAAGCCGAATACGGTGATTTTGGTTCCG |
| | | GTTACCCGTCTGATCCGCGTACTAAGAAATGG |
| | | CTGGAAGAATGGTATAGCAAACACGGCAATT |
| | | TCCCGCCGATCGTGCGTCGTACTTGGGATACT |
| | | GCAAAGAAAATCGAAGAAAAATTCAAACGTG |
| | | CGCAGCTGACCCTGGACAAGTTCTTCAAGAA |
| | | ACCTTGA |
| 96 | A107V | ATGAAAGTTGCAGGTGCAGATGAAGCTGGTC |
| | SEL28 | GTGGTCCAGTTATTGGTCCGCTGGTTATTGTT |
| | RNase H2 | GCTGCTGTTGTGGATGAAAACAGTCTCCCCAA |
| | | GCTGGAAGAGCTGAAAGTTAGAGACTCCAAA |
| | | AAGCTGACCCCGAAGCGACGTGAAAAACTGT |
| | | TCGATGAAATCGTAAAAGTACTGGATGATTAC |
| | | TCTGTGGTCATTGTGTCCCCGCAGGACATTGA |
| | | CGGTCGTAAGGGCAGCATGAACGAACTGGAG |
| | | GTAGAAAACTTCGTTAAAGCCCTGAATAGCCT |
| | | GAAAGTTAAGCCGGATGTTATTTACGTAGAT |
| | | GCCGCTGATGTTGATGAAGAACGTTTCGCTAG |
| | | AGAGCTTGGCGAGCGTCTGAACTTCGAAGCG |
| | | GAAGTTGTAGCCAAACATAAAGCGGATGACA |
| | | TCTTTCCGGTCGTATCCGCAGCCTCTATCCTG |
| | | GCAAAAGTTATCCGTGACCGCGAGATCGAAA |
| | | AGCTGAAAGCCGAATACGGTGATTTTGGTTCC |
| | | GGTTACCCGTCTGATCCGCGTACTAAGAAATG |
| | | GCTGGAAGAATGGTATAGCAAACACGGCAAT |
| | | TTCCCGCCGATCGTGCGTCGTACTTGGGATAC |
| | | TGCAAAGAAAATCGAAGAAAAATTCAAACGT |
| | | GCGCAGCTGACCCTGGACAAGTTCTTCAAGA |
| | | AACCTTGA |
| 97 | P13S/A107V | ATGAAAGTTGCAGGTGCAGATGAAGCTGGTC |
| | SEL28 | GTGGTTCTGTTATTGGTCCGCTGGTTATTGTT |
| | RNase H2 | GCTGCTGTTGTGGATGAAAACAGTCTCCCCAA |
| | | GCTGGAAGAGCTGAAAGTTAGAGACTCCAAA |
| | | AAGCTGACCCCGAAGCGACGTGAAAAACTGT |
| | | TCGATGAAATCGTAAAAGTACTGGATGATTAC |
| | | TCTGTGGTCATTGTGTCCCCGCAGGACATTGA |
| | | CGGTCGTAAGGGCAGCATGAACGAACTGGAG |
| | | GTAGAAAACTTCGTTAAAGCCCTGAATAGCCT |
| | | GAAAGTTAAGCCGGATGTTATTTACGTAGAT |
| | | GCCGCTGATGTTGATGAAGAACGTTTCGCTAG |
| | | AGAGCTTGGCGAGCGTCTGAACTTCGAAGCG |
| | | GAAGTTGTAGCCAAACATAAAGCGGATGACA |
| | | TCTTTCCGGTCGTATCCGCAGCCTCTATCCTG |
| | | GCAAAAGTTATCCGTGACCGCGAGATCGAAA |
| | | AGCTGAAAGCCGAATACGGTGATTTTGGTTCC |
| | | GGTTACCCGTCTGATCCGCGTACTAAGAAATG |
| | | GCTGGAAGAATGGTATAGCAAACACGGCAAT |
| | | TTCCCGCCGATCGTGCGTCGTACTTGGGATAC |
| | | TGCAAAGAAAATCGAAGAAAAATTCAAACGT |
| | | GCGCAGCTGACCCTGGACAAGTTCTTCAAGA |
| | | AACCTTGA |
| 98 | P13S | ATGAAAGTTGCAGGTATAGATGAAGCTGGTC |
| | SEL29 | GTGGTTCTGCTATTGGTCCGCTGGTTATTGTT |
| | RNase H2 | GCTGCTGTTGTGGATGAAAACAGTCTCCCCAA |
| | | GCTGGAAGAGCTGAAAGTTAGAGACTCCAAA |
| | | AAGCTGACCCCGGCGCAACGTGAAAAACTGT |
| | | TCGATGAAATCGTAAAAGTACTGGATGATTAC |
| | | TCTGTGGTCATTGTGTCCCCGCAGGACATTGA |
| | | CGGTCGTAAGGGCAGCATGAACGAACTGGAG |
| | | GTAGAAAACTTCGTTAAAGCCCTGAATAGCCT |
| | | GAAAGTTAAGCCGGATGTTATTTACGCTGATG |
| | | CCGCTGATGTTGATGAAGAACGTTTCGCTAGA |
| | | GAGCTTGGCGAGCGTCTGAACTTCGAAGCGA |
| | | AAGTTGTAGCCGAACATAAAGCGGATGCGAA |
| | | GTATGAGATCGTATCCGCAGCCTCTATCCTGG |
| | | CAAAAGTTATCCGTGACCGCGAGATCGAAAA |
| | | GCTGAAAGCCGAATACGGTGATTTTGGTTCCG |
| | | GTTACCCGTCTGATCCGCGTACTAGGGCATTC |
| | | CTGGAAAATTACTATAGAGAACACGGCGAAT |
| | | TCCCGCCGATCGTGCGTAAAGGTTGGAAGACT |
| | | CTAAAGAAAATCGCAGAAAAAGTCGAAAGTG |
| | | AGAAGAAGGCTGAGGAACGGCAGGCGACCCT |
| | | GGACAGGTACTTCAGGAAGGTTTGA |
| 99 | Q48R | ATGAAAGTTGCAGGTATAGATGAAGCTGGTC |
| | SEL29 | GTGGTCCAGCTATTGGTCCGCTGGTTATTGTT |
| | RNase H2 | GCTGCTGTTGTGGATGAAAACAGTCTCCCCAA |
| | | GCTGGAAGAGCTGAAAGTTAGAGACTCCAAA |
| | | AAGCTGACCCCGGCGCGCCGTGAAAAACTGT |
| | | TCGATGAAATCGTAAAAGTACTGGATGATTAC |
| | | TCTGTGGTCATTGTGTCCCCGCAGGACATTGA |
| | | CGGTCGTAAGGGCAGCATGAACGAACTGGAG |
| | | GTAGAAAACTTCGTTAAAGCCCTGAATAGCCT |
| | | GAAAGTTAAGCCGGATGTTATTTACGCTGATG |
| | | CCGCTGATGTTGATGAAGAACGTTTCGCTAGA |
| | | GAGCTTGGCGAGCGTCTGAACTTCGAAGCGA |
| | | AAGTTGTAGCCGAACATAAAGCGGATGCGAA |
| | | GTATGAGATCGTATCCGCAGCCTCTATCCTGG |
| | | CAAAAGTTATCCGTGACCGCGAGATCGAAAA |
| | | GCTGAAAGCCGAATACGGTGATTTTGGTTCCG |
| | | GTTACCCGTCTGATCCGCGTACTAGGGCATTC |
| | | CTGGAAAATTACTATAGAGAACACGGCGAAT |
| | | TCCCGCCGATCGTGCGTAAAGGTTGGAAGACT |
| | | CTAAAGAAAATCGCAGAAAAAGTCGAAAGTG |
| | | AGAAGAAGGCTGAGGAACGGCAGGCGACCCT |
| | | GGACAGGTACTTCAGGAAGGTTTGA |
| 100 | A107V | ATGAAAGTTGCAGGTATAGATGAAGCTGGTC |
| | SEL29 | GTGGTCCAGCTATTGGTCCGCTGGTTATTGTT |
| | RNase H2 | GCTGCTGTTGTGGATGAAAACAGTCTCCCCAA |
| | | GCTGGAAGAGCTGAAAGTTAGAGACTCCAAA |
| | | AAGCTGACCCCGGCGCAACGTGAAAAACTGT |
| | | TCGATGAAATCGTAAAAGTACTGGATGATTAC |
| | | TCTGTGGTCATTGTGTCCCCGCAGGACATTGA |
| | | CGGTCGTAAGGGCAGCATGAACGAACTGGAG |
| | | GTAGAAAACTTCGTTAAAGCCCTGAATAGCCT |
| | | GAAAGTTAAGCCGGATGTTATTTACGCTGATG |
| | | CCGTAGATGTTGATGAAGAACGTTTCGCTAG |
| | | AGAGCTTGGCGAGCGTCTGAACTTCGAAGCG |
| | | AAAGTTGTAGCCGAACATAAAGCGGATGCGA |
| | | AGTATGAGATCGTATCCGCAGCCTCTATCCTG |
| | | GCAAAAGTTATCCGTGACCGCGAGATCGAAA |
| | | AGCTGAAAGCCGAATACGGTGATTTTGGTTCC |
| | | GGTTACCCGTCTGATCCGCGTACTAGGGCATT |
| | | CCTGGAAAATTACTATAGAGAACACGGCGAA |
| | | TTCCCGCCGATCGTGCGTAAAGGTTGGAAGAC |
| | | TCTAAAGAAAATCGCAGAAAAAGTCGAAAGT |
| | | GAGAAGAAGGCTGAGGAACGGCAGGCGACCC |
| | | TGGACAGGTACTTCAGGAAGGTTTGA |
| 101 | P13S/A107V | ATGAAAGTTGCAGGTATAGATGAAGCTGGTC |
| | SEL29 | GTGGTTCTGCTATTGGTCCGCTGGTTATTGTT |
| | RNase H2 | GCTGCTGTTGTGGATGAAAACAGTCTCCCCAA |
| | | GCTGGAAGAGCTGAAAGTTAGAGACTCCAAA |
| | | AAGCTGACCCCGGCGCAACGTGAAAAACTGT |
| | | TCGATGAAATCGTAAAAGTACTGGATGATTAC |
| | | TCTGTGGTCATTGTGTCCCCGCAGGACATTGA |
| | | CGGTCGTAAGGGCAGCATGAACGAACTGGAG |
| | | GTAGAAAACTTCGTTAAAGCCCTGAATAGCCT |
| | | GAAAGTTAAGCCGGATGTTATTTACGCTGATG |
| | | CCGTAGATGTTGATGAAGAACGTTTCGCTAG |
| | | AGAGCTTGGCGAGCGTCTGAACTTCGAAGCG |
| | | AAAGTTGTAGCCGAACATAAAGCGGATGCGA |
| | | AGTATGAGATCGTATCCGCAGCCTCTATCCTG |
| | | GCAAAAGTTATCCGTGACCGCGAGATCGAAA |
| | | AGCTGAAAGCCGAATACGGTGATTTTGGTTCC |
| | | GGTTACCCGTCTGATCCGCGTACTAGGGCATT |
| | | CCTGGAAAATTACTATAGAGAACACGGCGAA |
| | | TTCCCGCCGATCGTGCGTAAAGGTTGGAAGAC |
| | | TCTAAAGAAAATCGCAGAAAAAGTCGAAAGT |
| | | GAGAAGAAGGCTGAGGAACGGCAGGCGACCC |
| | | TGGACAGGTACTTCAGGAAGGTTTGA |

¹Location of mutations are shown in bold and underlined. The stop codon is shown in italics.

### REFERENCES CITED

Joseph R Dobosy, Scott D Rose, Kristin R Beltz, Susan M Rupp, Kristy M Powers, Mark A Behlke and Joseph A Walder. RNase H-dependent PCR (rhPCR): improved specificity and single nucleotide polymorphism detection using blocked cleavable primers. BMC Biotechnology (2011), 11:80.
Ayumu Muroya, Daisuke Tsuchiya, Momoyo Ishikawa, Mitsuru Haruki, Masaaki Morikawa, Shigenori Kanaya, and Kosuke Morikawa. Catalytic center of an archaeal type 2 ribonuclease H as revealed by X-ray crystallographic and mutational analyses. Protein Science (2001), 10:707-714.
Monika P. Rychlik, Hyongi Chon, Susana M. Cerritelli, Paulina Klimek, Robert J. Crouch, and Marcin Nowotny. Crystal Structures of RNase H2 in Complex with Nucleic Acid Reveal the Mechanism of RNA-DNA Junction Recognition and Cleavage. Molecular Cell (2010), 40:658-670.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A kit for producing an extended primer, comprising at least one container providing a hybrid RNase H2 protein selected from the group consisting of one of the following:
a hybrid RNase H2 protein comprising fragments of amino acid sequences from Pyrococcus abyssi (P. a.), Thermococcus kodakarensis (T. kod), and Pyrococcus furiosus organisms;
a hybrid RNase H2 protein comprises amino acid residues 26-40 and residues 100-120 of T. kod RNase H2;
a hybrid RNase H2 protein is selected from SEQ ID NOs.: 2 and 3; and
a hybrid RNase H2 protein is selected from SEQ ID NOs.: 14-20.

2. The kit according to claim 2, further comprising one or more additional containers selected from the group consisting of:
(a) a container providing a primer hybridizable, under primer extension conditions, to a predetermined polynucleotide template;
(b) a container providing nucleoside triphosphates;
(c) a container providing a buffer suitable for primer extension and
(d) a DNA polymerase.

3. The kit according to claim 2, wherein the DNA polymerase comprises a high-fidelity archaeal DNA polymerase.

4. The kit according claims 1, further comprising a container containing a blocked-cleavable primer.

5. A kit for performing amplification of a target DNA sequence, comprising a reaction buffer that includes an RNase H2 of claim 1 and a high-fidelity archaeal DNA polymerase.

6. The kit of claim 5, further comprising one or more oligonucleotide primers, wherein at least one oligonucleotide primer has a cleavage domain, which is cleavable by an RNase H2 enzyme, positioned 5′ of a blocking group, said blocking group linked at or near the 3′-end of the oligonucleotide primer wherein said blocking group prevents primer extension and/or inhibits the oligonucleotide primer from serving as a template for DNA synthesis.

7. The kit of claim 6, wherein the blocking group comprises one member selected from the group consisting of RDDDDx, RDDDDMx, RDxxD, RDxxDM, RDDDDxxD, RDDDDxxDM and DxxD, wherein R is an RNA residue, D is a DNA residue, M is a mismatched residue and x is a C3 spacer or other moiety blocking extension by a DNA polymerase.


SEQUENCE LISTING (101 sequences)

SEQ ID NO: 1 - protein, 255 aa, synthetic construct
  REGION 1..255: note = SYNTHETIC SEQUENCE
MKVAGADEAGRGPVIGPLVIVAAVVEEDKIRSLTKLGVKDSKQLTPAQREKLFDEIVKVLDDYSVVIVSPQDIDGRKGSMNELEVENFVKALNSLKVKPEVIYIDSADVKAERFAENIRSRLAYEAKVVAEHKADAKYEIVSAASILAKVIRDREIEKLKAEYGDFGSGYPSDPRTKKWLEEWYSKHGNFPPIVRRTWDTAKKIEEKFKRAQLTLDNFLKRFRNKLAAALEIKRASQPELAPEDPEDVEHHHHHH

SEQ ID NO: 2 - protein, 253 aa, synthetic construct
  REGION 1..253: note = SYNTHETIC SEQUENCE
MKVAGADEAGRGPVIGPLVIVAAVVDENSLPKLEELKVRDSKKLTPKRREKLFDEIVKVLDDYSVVIVSPQDIDGRKGSMNELEVENFVKALNSLKVKPDVIYADAADVDEERFARELGERLNFEAEVVAKHKADDIFPVVSAASILAKVIRDREIEKLKAEYGDFGSGYPSDPRTKKWLEEWYSKHGNFPPIVRRTWDTAKKIEEKFKRAQLTLDKFFKKPKLAAALEIKRASQPELAPEDPEDVEHHHHHH

SEQ ID NO: 3 - protein, 259 aa, synthetic construct
  REGION 1..259: note = SYNTHETIC SEQUENCE
MKVAGIDEAGRGPAIGPLVIVAAVVDENSLPKLEELKVRDSKKLTPAQREKLFDEIVKVLDDYSVVIVSPQDIDGRKGSMNELEVENFVKALNSLKVKPDVIYADAADVDEERFARELGERLNFEAKVVAEHKADAKYEIVSAASILAKVIRDREIEKLKAEYGDFGSGYPSDPRTRAFLENYYREHGEFPPIVRKGWKTLKKIAEKVESEKKAEERQATLDRYFRKVKLAAALEIKRASQPELAPEDPEDVEHHHHHH

SEQ ID NO: 4 - DNA, 54 bp, synthetic construct
  misc_feature 1..54: note = SYNTHETIC SEQUENCE
  source 1..54: mol_type = other DNA
ggtgcagatgaagctggtcgtggttctgttattggtccgctggttattgttgct

SEQ ID NO: 5 - DNA, 54 bp, synthetic construct
  misc_feature 1..54: note = SYNTHETIC SEQUENCE
  source 1..54: mol_type = other DNA
agcaacaataaccagcggaccaataacagaaccacgaccagcttcatctgcacc

SEQ ID NO: 6 - DNA, 57 bp, synthetic construct
  misc_feature 1..57: note = SYNTHETIC SEQUENCE
  source 1..57: mol_type = other DNA
aagccggatgttatttacgctgatgccgtagatgttgatgaagaacgtttcgctaga

SEQ ID NO: 7 - DNA, 57 bp, synthetic construct
  misc_feature 1..57: note = SYNTHETIC SEQUENCE
  source 1..57: mol_type = other DNA
tctagcgaaacgttcttcatcaacatctacggcatcagcgtaaataacatccggctt

SEQ ID NO: 8 - DNA, 54 bp, synthetic construct
  misc_feature 1..54: note = SYNTHETIC SEQUENCE
  source 1..54: mol_type = other DNA
ggtatagatgaagctggtcgtggttctgctattggtccgctggttattgttgct

SEQ ID NO: 9 - DNA, 54 bp, synthetic construct
  misc_feature 1..54: note = SYNTHETIC SEQUENCE
  source 1..54: mol_type = other DNA
agcaacaataaccagcggaccaatagcagaaccacgaccagcttcatctatacc

SEQ ID NO: 10 - DNA, 53 bp, synthetic construct
  misc_feature 1..53: note = SYNTHETIC SEQUENCE
  source 1..53: mol_type = other DNA
agactccaaaaagctgaccccggcgcgccgtgaaaaactgttcgatgaaatcg

SEQ ID NO: 11 - DNA, 53 bp, synthetic construct
  misc_feature 1..53: note = SYNTHETIC SEQUENCE
  source 1..53: mol_type = other DNA
cgatttcatcgaacagtttttcacggcgcgccggggtcagctttttggagtct

SEQ ID NO: 12 - DNA, 57 bp, synthetic construct; identical to SEQ ID NO: 6
  misc_feature 1..57: note = SYNTHETIC SEQUENCE
  source 1..57: mol_type = other DNA

SEQ ID NO: 13 - DNA, 57 bp, synthetic construct; identical to SEQ ID NO: 7
  misc_feature 1..57: note = SYNTHETIC SEQUENCE
  source 1..57: mol_type = other DNA

SEQ ID NO: 14 - protein, 253 aa, synthetic construct
  REGION 1..253: note = SYNTHETIC SEQUENCE
MKVAGADEAGRGSVIGPLVIVAAVVDENSLPKLEELKVRDSKKLTPKRREKLFDEIVKVLDDYSVVIVSPQDIDGRKGSMNELEVENFVKALNSLKVKPDVIYADAADVDEERFARELGERLNFEAEVVAKHKADDIFPVVSAASILAKVIRDREIEKLKAEYGDFGSGYPSDPRTKKWLEEWYSKHGNFPPIVRRTWDTAKKIEEKFKRAQLTLDKFFKKPKLAAALEIKRASQPELAPEDPEDVEHHHHHH

SEQ ID NO: 15 - protein, 253 aa, synthetic construct
  REGION 1..253: note = SYNTHETIC SEQUENCE
MKVAGADEAGRGPVIGPLVIVAAVVDENSLPKLEELKVRDSKKLTPKRREKLFDEIVKVLDDYSVVIVSPQDIDGRKGSMNELEVENFVKALNSLKVKPDVIYADAVDVDEERFARELGERLNFEAEVVAKHKADDIFPVVSAASILAKVIRDREIEKLKAEYGDFGSGYPSDPRTKKWLEEWYSKHGNFPPIVRRTWDTAKKIEEKFKRAQLTLDKFFKKPKLAAALEIKRASQPELAPEDPEDVEHHHHHH

SEQ ID NO: 16 - protein, 253 aa, synthetic construct
  REGION 1..253: note = SYNTHETIC SEQUENCE
MKVAGADEAGRGSVIGPLVIVAAVVDENSLPKLEELKVRDSKKLTPKRREKLFDEIVKVLDDYSVVIVSPQDIDGRKGSMNELEVENFVKALNSLKVKPDVIYADAVDVDEERFARELGERLNFEAEVVAKHKADDIFPVVSAASILAKVIRDREIEKLKAEYGDFGSGYPSDPRTKKWLEEWYSKHGNFPPIVRRTWDTAKKIEEKFKRAQLTLDKFFKKPKLAAALEIKRASQPELAPEDPEDVEHHHHHH

SEQ ID NO: 17 - protein, 259 aa, synthetic construct
  REGION 1..259: note = SYNTHETIC SEQUENCE
MKVAGIDEAGRGSAIGPLVIVAAVVDENSLPKLEELKVRDSKKLTPAQREKLFDEIVKVLDDYSVVIVSPQDIDGRKGSMNELEVENFVKALNSLKVKPDVIYADAADVDEERFARELGERLNFEAKVVAEHKADAKYEIVSAASILAKVIRDREIEKLKAEYGDFGSGYPSDPRTRAFLENYYREHGEFPPIVRKGWKTLKKIAEKVESEKKAEERQATLDRYFRKVKLAAALEIKRASQPELAPEDPEDVEHHHHHH

SEQ ID NO: 18 - protein, 259 aa, synthetic construct
  REGION 1..259: note = SYNTHETIC SEQUENCE
MKVAGIDEAGRGPAIGPLVIVAAVVDENSLPKLEELKVRDSKKLTPARREKLFDEIVKVLDDYSVVIVSPQDIDGRKGSMNELEVENFVKALNSLKVKPDVIYADAADVDEERFARELGERLNFEAKVVAEHKADAKYEIVSAASILAKVIRDREIEKLKAEYGDFGSGYPSDPRTRAFLENYYREHGEFPPIVRKGWKTLKKIAEKVESEKKAEERQATLDRYFRKVKLAAALEIKRASQPELAPEDPEDVEHHHHHH

SEQ ID NO: 19 - protein, 259 aa, synthetic construct
  REGION 1..259: note = SYNTHETIC SEQUENCE
MKVAGIDEAGRGPAIGPLVIVAAVVDENSLPKLEELKVRDSKKLTPAQREKLFDEIVKVLDDYSVVIVSPQDIDGRKGSMNELEVENFVKALNSLKVKPDVIYADAVDVDEERFARELGERLNFEAKVVAEHKADAKYEIVSAASILAKVIRDREIEKLKAEYGDFGSGYPSDPRTRAFLENYYREHGEFPPIVRKGWKTLKKIAEKVESEKKAEERQATLDRYFRKVKLAAALEIKRASQPELAPEDPEDVEHHHHHH

SEQ ID NO: 20 - protein, 259 aa, synthetic construct
  REGION 1..259: note = SYNTHETIC SEQUENCE
MKVAGIDEAGRGSAIGPLVIVAAVVDENSLPKLEELKVRDSKKLTPAQREKLFDEIVKVLDDYSVVIVSPQDIDGRKGSMNELEVENFVKALNSLKVKPDVIYADAVDVDEERFARELGERLNFEAKVVAEHKADAKYEIVSAASILAKVIRDREIEKLKAEYGDFGSGYPSDPRTRAFLENYYREHGEFPPIVRKGWKTLKKIAEKVESEKKAEERQATLDRYFRKVKLAAALEIKRASQPELAPEDPEDVEHHHHHH

SEQ ID NO: 21 - DNA, 22 bp, synthetic construct
  misc_feature 1..22: note = SYNTHETIC SEQUENCE
  source 1..22: mol_type = other DNA
cagcctcatccaaaagaggaaa

SEQ ID NO: 22 - DNA, 28 bp, synthetic construct
  misc_feature 1..28: note = SYNTHETIC SEQUENCE
  misc_feature 23: note = RIBONUCLEOTIDE
  misc_feature 28: note = 3'-PROPRIETARY BLOCKER GROUP
  source 1..28: mol_type = other DNA
cagcctcatccaaaagaggaaacaggag

SEQ ID NO: 23 - DNA, 21 bp, synthetic construct
  misc_feature 1..21: note = SYNTHETIC SEQUENCE
  source 1..21: mol_type = other DNA
ctcactctaaaccccagcatt

SEQ ID NO: 24 - DNA, 41 bp, synthetic construct
  misc_feature 1..41: note = SYNTHETIC SEQUENCE
  misc_feature 1: note = 5'- Iowa Black FLUORESCENT QUENCHER
  misc_feature 36: note = RIBONUCLEOTIDE
  misc_feature 41: note = 3'-(6-FAM) FLUORESCENT LABEL
  source 1..41: mol_type = other DNA
tataagctaccagcatggtttttccatgctggtagcttata

SEQ ID NO: 25 - DNA, 41 bp, synthetic construct; identical to SEQ ID NO: 24
  misc_feature 1..41: note = SYNTHETIC SEQUENCE
  misc_feature 36: note = RIBONUCLEOTIDE
  source 1..41: mol_type = other DNA

SEQ ID NO: 26 - DNA, 23 bp, synthetic construct
  misc_feature 1..23: note = SYNTHETIC SEQUENCE
  source 1..23: mol_type = other DNA
agctctgcccaaagattaccctg

SEQ ID NO: 27 - DNA, 22 bp, synthetic construct
  misc_feature 1..22: note = SYNTHETIC SEQUENCE
  source 1..22: mol_type = other DNA
ctgagcttcatgcctttactgt

SEQ ID NO: 28 - DNA, 28 bp, synthetic construct
  misc_feature 1..28: note = SYNTHETIC SEQUENCE
  misc_feature 23: note = RIBONCLEOTIDE
  misc_feature 28: note = 3'-C3 spacer (propanediol) blocker group
  source 1..28: mol_type = other DNA
ctgagcttcatgcctttactgtaccccc

SEQ ID NO: 29 - DNA, 28 bp, synthetic construct
  misc_feature 1..28: note = SYNTHETIC SEQUENCE
  misc_feature 23: note = RIBONUCLEOTIDE
  misc_feature 28: note = 3'-C3 spacer (propanediol) blocker group
  source 1..28: mol_type = other DNA
ctgagcttcatgcctttactgtcccccc

SEQ ID NO: 30 - DNA, 28 bp, synthetic construct
  misc_feature 1..28: note = SYNTHETIC SEQUENCE
  misc_feature 23: note = RIBONUCLEOTIDE
  misc_feature 28: note = 3'-C3 spacer (propanediol) blocker group
  source 1..28: mol_type = other DNA
ctgagcttcatgcctttactgtgccccc

SEQ ID NO: 31 - DNA, 28 bp, synthetic construct
  misc_feature 1..28: note = SYNTHETIC SEQUENCE
  misc_feature 23: note = RIBONUCLEOTIDE
  misc_feature 28: note = 3'-C3 spacer (propanediol) blocker group
  source 1..28: mol_type = other DNA
ctgagcttcatgcctttactgttccccc

SEQ ID NO: 32 - DNA, 103 bp, synthetic construct
  misc_feature 1..103: note = SYNTHETIC SEQUENCE
  source 1..103: mol_type = other DNA
agctctgcccaaagattaccctgacagctaagtggcagtggaagttggcctcagaagtagtggccagctgtgtgtcggggaacagtaaaggcatgaagctcag

SEQ ID NO: 33 - DNA, 103 bp, synthetic construct
  misc_feature 1..103: note = SYNTHETIC SEQUENCE
  source 1..103: mol_type = other DNA
agctctgcccaaagattaccctgacagctaagtggcagtggaagttggcctcagaagtagtggccagctgtgtgtcggggcacagtaaaggcatgaagctcag

SEQ ID NO: 34 - DNA, 103 bp, synthetic construct
  misc_feature 1..103: note = SYNTHETIC SEQUENCE
  source 1..103: mol_type = other DNA
agctctgcccaaagattaccctgacagctaagtggcagtggaagttggcctcagaagtagtggccagctgtgtgtcgggggacagtaaaggcatgaagctcag

SEQ ID NO: 35 - DNA, 103 bp, synthetic construct
  misc_feature 1..103: note = SYNTHETIC SEQUENCE
  source 1..103: mol_type = other DNA
agctctgcccaaagattaccctgacagctaagtggcagtggaagttggcctcagaagtagtggccagctgtgtgtcggggtacagtaaaggcatgaagctcag

SEQ ID NO: 36 - DNA, 22 bp, synthetic construct
  misc_feature 1..22: note = SYNTHETIC SEQUENCE
  source 1..22: mol_type = other DNA
gtgattttggtctagctacagt

SEQ ID NO: 37 - DNA, 23 bp, synthetic construct
  misc_feature 1..23: note = SYNTHETIC SEQUENCE
  source 1..23: mol_type = other DNA
cctcaattcttaccatccacaaa

SEQ ID NO: 38 - DNA, 28 bp, synthetic construct
  misc_feature 1..28: note = SYNTHETIC SEQUENCE
  misc_feature 23: note = RIBONUCLEOTIDE
  misc_feature 28: note = 3'-C3 spacer (propanediol) blocker group
  source 1..28: mol_type = other DNA
gtgattttggtctagctacagtgaaatg

SEQ ID NO: 39 - DNA, 28 bp, synthetic construct
  misc_feature 1..28: note = SYNTHETIC SEQUENCE
  misc_feature 23: note = RIBONUCLEOTIDE
  misc_feature 28: note = 3'-C3 spacer (propanediol) blocker group
  source 1..28: mol_type = other DNA
gtgattttggtctagctacagagaaatg

SEQ ID NO: 40 - DNA, 401 bp, synthetic construct
  misc_feature 1..401: note = SYNTHETIC SEQUENCE
  source 1..401: mol_type = other DNA
taagaggaaagatgaagtactatgttttaaagaatattatattacagaattatagaaattagatctcttacctaaactcttcataatgcttgctctgataggaaaatgagatctactgttttcctttacttactacacctcagatatatttcttcatgaagacctcacagtaaaaataggtgattttggtctagctacagtgaaatctcgatggagtgggtcccatcagtttgaacagttgtctggatccattttgtggatggtaagaattgaggctatttttccactgattaaatttttggccctgagatgctgctgagttactagaaagtcattgaaggtctcaactatagtattttcatagttcccagtattcacaaaaatcagtgttcttatttttt

SEQ ID NO: 41 - DNA, 401 bp, synthetic construct
  misc_feature 1..401: note = SYNTHETIC SEQUENCE
  source 1..401: mol_type = other DNA
taagaggaaagatgaagtactatgttttaaagaatattatattacagaattatagaaattagatctcttacctaaactcttcataatgcttgctctgataggaaaatgagatctactgttttcctttacttactacacctcagatatatttcttcatgaagacctcacagtaaaaataggtgattttggtctagctacagagaaatctcgatggagtgggtcccatcagtttgaacagttgtctggatccattttgtggatggtaagaattgaggctatttttccactgattaaatttttggccctgagatgctgctgagttactagaaagtcattgaaggtctcaactatagtattttcatagttcccagtattcacaaaaatcagtgttcttatttttt

SEQ ID NO: 42 - DNA, 23 bp, synthetic construct
  misc_feature 1..23: note = SYNTHETIC SEQUENCE
  source 1..23: mol_type = other DNA
ggcagattttcttctgcaccgcg

SEQ ID NO: 43 - DNA, 27 bp, synthetic construct
  misc_feature 1..27: note = SYNTHETIC SEQUENCE
  misc_feature 23: note = RIBONUCLEOTIDE
  misc_feature 25: note = INTERNUCLEOTIDE C3 PROPANE DIOL SPACER
  misc_feature 27: note = 3'-C3 PROPANE DIOL SPACER
  source 1..27: mol_type = other DNA
ggcagattttcttctgcaccgcggttc

SEQ ID NO: 44 - DNA, 27 bp, synthetic construct
  misc_feature 1..27: note = SYNTHETIC SEQUENCE
  source 1..27: mol_type = other DNA
tcccgtcgagcaccagcaattttactc

SEQ ID NO: 45 - DNA, 24 bp, synthetic construct
  misc_feature 1..24: note = SYNTHETIC SEQUENCE
  source 1..24: mol_type = other DNA
ctttggataaggaagaagccaact

SEQ ID NO: 46 - DNA, 28 bp, synthetic construct
  misc_feature 1..28: note = SYNTHETIC SEQUENCE
  misc_feature 24: note = RIBONUCLEOTIDE
  misc_feature 26: note = INTERNUCLEOTIDE C2 PROPANE DIOL
  misc_feature 28: note = 3'-C2 PROPANE DIOL
  source 1..28: mol_type = other DNA
ctttggataaggaagaagccaactgcag

SEQ ID NO: 47 - DNA, 28 bp, synthetic construct
  misc_feature 1..28: note = SYNTHETIC SEQUENCE
  source 1..28: mol_type = other DNA
gggagcttgaaatgaacaaggtgagaag

SEQ ID NO: 48 - DNA, 46 bp, synthetic construct
  misc_feature 1..46: note = SYNTHETIC SEQUENCE
  source 1..46: mol_type = other DNA
cagccagccgcagcacgttcgctcataggagatatggtagagccgc

SEQ ID NO: 49 - DNA, 51 bp, synthetic construct
  misc_feature 1..51: note = SYNTHETIC SEQUENCE
  source 1..51: mol_type = other DNA
gagagaatttgtaccacctcccaccgggcacatagcagtcctagggacagt

SEQ ID NO: 50 - DNA, 22 bp, synthetic construct
  misc_feature 1..22: note = SYNTHETIC SEQUENCE
  source 1..22: mol_type = other DNA
ggcttggctctgctaacacgtt

SEQ ID NO: 51 - DNA, 22 bp, synthetic construct
  misc_feature 1..22: note = SYNTHETIC SEQUENCE
  source 1..22: mol_type = other DNA
ggacgtttgtaatgtccgctcc

SEQ ID NO: 52 - DNA, 20 bp, synthetic construct
  misc_feature 1..20: note = SYNTHETIC SEQUENCE
  misc_feature 1: note = 5'-(6-carboxyfluorescein) MOIETY
  misc_feature 11: note = RIBONUCLEOTIDE
  misc_feature 20: note = 3'-( Iowa Black FQ fluorescence quencher) MOIETY
  source 1..20: mol_type = other DNA
acgtgctgcggctggctggt

SEQ ID NO: 53 - DNA, 48 bp, synthetic construct
  misc_feature 1..48: note = SYNTHETIC SEQUENCE
  source 1..48: mol_type = other DNA
ccaaagagtaaagtccttctctctcgagagactgttggcccttgaagg

SEQ ID NO: 54 - DNA, 47 bp, synthetic construct
  misc_feature 1..47: note = SYNTHETIC SEQUENCE
  source 1..47: mol_type = other DNA
gtgttgatgttatccaccttttgtggactaggaaaacagatcaatag

SEQ ID NO: 55 - DNA, 20 bp, synthetic construct
  misc_feature 1..20: note = SYNTHETIC SEQUENCE
  source 1..20: mol_type = other DNA
taatcctggaactccggtgc

SEQ ID NO: 56 - DNA, 23 bp, synthetic construct
  misc_feature 1..23: note = SYNTHETIC SEQUENCE
  source 1..23: mol_type = other DNA
tttatgccaattaacattttgac

SEQ ID NO: 57 - DNA, 24 bp, synthetic construct
  misc_feature 1..24: note = SYNTHETIC SEQUENCE
  misc_feature 1: note = 5'-(6-carboxyfluorescein) MOIETY
  misc_feature 14: note = RIBONCLEOTIDE
  misc_feature 24: note = 3'-(Iowa Black FQ fluorescence quencher) MOIETY
  source 1..24: mol_type = other DNA
cctccctgtggatgagagagaagg

SEQ ID NO: 58 - DNA, 52 bp, synthetic construct
  misc_feature 1..52: note = SYNTHETIC SEQUENCE
  misc_feature 47: note = RIBONUCLEOTIDE
  misc_feature 52: note = 3'-C3 PROPANE DIOL
  source 1..52: mol_type = other DNA
cagccagccgcagcacgttcgctcataggagatatggtagagccgcagacag

SEQ ID NO: 59 - DNA, 57 bp, synthetic construct
  misc_feature 1..57: note = SYNTHETIC SEQUENCE
  misc_feature 52: note = RIBONUCLEOTIDE
  misc_feature 57: note = 3'-C3 PROPANE DIOL
  source 1..57: mol_type = other DNA
gagagaatttgtaccacctcccaccgggcacatagcagtcctagggacagtggcgtt

SEQ ID NO: 60 - DNA, 28 bp, synthetic construct
  misc_feature 1..28: note = SYNTHETIC SEQUENCE
  misc_feature 23: note = RIBONUCLEOTIDE
  misc_feature 28: note = 3'-C3 PROPANE DIOL
  source 1..28: mol_type = other DNA
ggcttggctctgctaacacgttgctcaa

SEQ ID NO: 61 - DNA, 28 bp, synthetic construct
  misc_feature 1..28: note = SYNTHETIC SEQUENCE
  misc_feature 24: note = RIBONUCLEOTIDE
  misc_feature 28: note = 3'-C3 PROPANE DIOL
  source 1..28: mol_type = other DNA
ggacgtttgtaatgtccgctccggcact

SEQ ID NO: 62 - DNA, 20 bp, synthetic construct
  misc_feature 1..20: note = SYNTHETIC SEQUENCE
  misc_feature 1: note = 5'-(6-carboxyfluorescein) MOIETY
  misc_feature 11: note = RIBONUCLEOTIDE
  misc_feature 20: note = 3'-(Iowa Black FQ fluorescence quencher) MOIETY
  source 1..20: mol_type = other DNA
acgtgctgcggctggctggt

SEQ ID NO: 63 - DNA, 54 bp, synthetic construct
  misc_feature 1..54: note = SYNTHETIC SEQUENCE
  misc_feature 49: note = RIBONUCLEOTIDE
  misc_feature 54: note = 3'-C3 PROPANE DIOL
  source 1..54: mol_type = other DNA
ccaaagagtaaagtccttctctctcgagagactgttggcccttgaaggagagca

SEQ ID NO: 64 - DNA, 53 bp, synthetic construct
  misc_feature 1..53: note = SYNTHETIC SEQUENCE
  misc_feature 48: note = RIBONUCLEOTIDE
  misc_feature 53: note = 3'-C3 PROPANE DIOL
  source 1..53: mol_type = other DNA
gtgttgatgttatccaccttttgtggactaggaaaacagatcaatagataagc

SEQ ID NO: 65 - DNA, 26 bp, synthetic construct
  misc_feature 1..26: note = SYNTHETIC SEQUENCE
  misc_feature 21: note = RIBONUCLEOTIDE
  misc_feature 26: note = 3'-C3 PROPANE DIOL
  source 1..26: mol_type = other DNA
taatcctggaactccggtgctaaggt

SEQ ID NO: 66 - DNA, 29 bp, synthetic construct
  misc_feature 1..29: note = SYNTHETIC SEQUENCE
  misc_feature 24: note = RIBONUCLEOTIDE
  misc_feature 29: note = 3'-C3 PROPANE DIOL
  source 1..29: mol_type = other DNA
tttatgccaattaacattttgactttatt

SEQ ID NO: 67 - DNA, 24 bp, synthetic construct
  misc_feature 1..24: note = SYNTHETIC SEQUENCE
  misc_feature 1: note = 5'-(6-carboxyfluorescein) MOIETY
  misc_feature 14: note = RIBONUCLEOTIDE
  misc_feature 24: note = 3'-(Iowa Black FQ fluorescence quencher) MOIETY
  source 1..24: mol_type = other DNA
cctccctgtggatgagagagaagg

SEQ ID NO: 68 - DNA, 50 bp, synthetic construct
  misc_feature 1..50: note = SYNTHETIC SEQUENCE
  misc_feature 47: note = RIBONUCLEOTIDE
  misc_feature 48: note = 2 C3 PROPANE DIOL INTERNUCLEOTIDE LINKAGES
  source 1..50: mol_type = other DNA
cagccagccgcagcacgttcgctcataggagatatggtagagccgcagag

SEQ ID NO: 69 - DNA, 55 bp, synthetic construct
  misc_feature 1..55: note = SYNTHETIC SEQUENCE
  misc_feature 52: note = RIBONUCLEOTIDE
  misc_feature 53: note = 2 C3 PROPANE DIOL INTERNUCLEOTIDE LINKAGES
  source 1..55: mol_type = other DNA
gagagaatttgtaccacctcccaccgggcacatagcagtcctagggacagtggtt

SEQ ID NO: 70 - DNA, 26 bp, synthetic construct
  misc_feature 1..26: note = SYNTHETIC SEQUENCE
  misc_feature 23: note = RIBONUCLEOTIDE
  misc_feature 24: note = 2 C3 PROPANE DIOL INTERNUCLEOTIDE LINKAGES
  source 1..26: mol_type = other DNA
ggcttggctctgctaacacgttgcaa

SEQ ID NO: 71 - DNA, 26 bp, synthetic construct
  misc_feature 1..26: note = SYNTHETIC SEQUENCE
  misc_feature 23: note = RIBONUCLEOTIDE
  misc_feature 24: note = 2 C3 PROPANE DIOL INTERNUCLEOTIDE LINKAGES
  source 1..26: mol_type = other DNA
ggacgtttgtaatgtccgctccggct

SEQ ID NO: 72 - DNA, 20 bp, synthetic construct
  misc_feature 1..20: note = SYNTHETIC SEQUENCE
  misc_feature 1: note = 5'-(6-carboxyfluorescein) MOIETY
  misc_feature 11: note = RIBONUCLEOTIDE
  misc_feature 20: note = 3'-(Iowa Black FQ fluorescence quencher) MOIETY
  source 1..20: mol_type = other DNA
acgtgctgcggctggctggt

SEQ ID NO: 73 - DNA, 52 bp, synthetic construct
  misc_feature 1..52: note = SYNTHETIC SEQUENCE
  misc_feature 49: note = RIBONUCLEOTIDE
  misc_feature 50: note = 2 C3 PROPANE DIOL INTERNUCLEOTIDE LINKAGES
  source 1..52: mol_type = other DNA
ccaaagagtaaagtccttctctctcgagagactgttggcccttgaaggagca

SEQ ID NO: 74 - DNA, 51 bp, synthetic construct
  misc_feature 1..51: note = SYNTHETIC SEQUENCE
  misc_feature 48: note = RIBONUCLEOTIDE
  misc_feature 49: note = 2 C3 PROPANE DIOL INTERNUCLEOTIDE LINKAGES
  source 1..51: mol_type = other DNA
gtgttgatgttatccaccttttgtggactaggaaaacagatcaatagatgc

SEQ ID NO: 75 - DNA, 24 bp, synthetic construct
  misc_feature 1..24: note = SYNTHETIC SEQUENCE
  misc_feature 21: note = RIBONUCLEOTIDE
  misc_feature 22: note = 2 C3 PROPANE DIOL INTERNUCLEOTIDE LINKAGES
  source 1..24: mol_type = other DNA
taatcctggaactccggtgctagt

SEQ ID NO: 76 - DNA, 27 bp, synthetic construct
  misc_feature 1..27: note = SYNTHETIC SEQUENCE
  misc_feature 24: note = RIBONUCLEOTIDE
  misc_feature 25: note = 2 C3 PROPANE DIOL INTERNUCLEOTIDE LINKAGES
  source 1..27: mol_type = other DNA
tttatgccaattaacattttgactttt

SEQ ID NO: 77 - DNA, 24 bp, synthetic construct
  misc_feature 1..24: note = SYNTHETIC SEQUENCE
  misc_feature 1: note = 5'-(6-carboxyfluorescein) MOIETY
  misc_feature 14: note = RIBONUCLEOTIDE
  misc_feature 24: note = 3'-(Iowa Black FQ fluorescence quencher) MOIETY
  source 1..24: mol_type = other DNA
cctccctgtggatgagagagaagg

SEQ ID NO: 78 - DNA, 768 bp, synthetic construct
  misc_feature 1..768: note = SYNTHETIC SEQUENCE
  source 1..768: mol_type = other DNA
atgaaagttgcaggtgcagatgaagctggtcgtggtccagttattggtccgctggttattgttgctgctgttgtggaggaagacaaaatccgctctctgactaagctgggtgttaaagactccaaacagctgaccccggcgcaacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggaagttatttacattgattccgctgatgttaaagctgaacgtttcgctgaaaacattcgcagccgtctggcgtacgaagcgaaagttgtagccgaacataaagcggatgcgaagtatgagatcgtatccgcagcctctatcctggcaaaagttatccgtgaccgcgagatcgaaaagctgaaagccgaatacggtgattttggttccggttacccgtctgatccgcgtactaagaaatggctggaagaatggtatagcaaacacggcaatttcccgccgatcgtgcgtcgtacttgggatactgcaaagaaaatcgaagaaaaattcaaacgtgcgcagctgaccctggacaacttcctgaagcgttttcgcaacaagcttgcggccgcactcgagatcaaacgggctagccagccagaactcgccccggaagaccccgaggatgtcgagcaccaccaccaccaccactga

SEQ ID NO: 79 - DNA, 762 bp, synthetic construct
  misc_feature 1..762: note = SYNTHETIC SEQUENCE
  source 1..762: mol_type = other DNA
atgaaagttgcaggtgcagatgaagctggtcgtggtccagttattggtccgctggttattgttgctgctgttgtggatgaaaacagtctccccaagctggaagagctgaaagttagagactccaaaaagctgaccccgaagcgacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgctgatgccgctgatgttgatgaagaacgtttcgctagagagcttggcgagcgtctgaacttcgaagcggaagttgtagccaaacataaagcggatgacatctttccggtcgtatccgcagcctctatcctggcaaaagttatccgtgaccgcgagatcgaaaagctgaaagccgaatacggtgattttggttccggttacccgtctgatccgcgtactaagaaatggctggaagaatggtatagcaaacacggcaatttcccgccgatcgtgcgtcgtacttgggatactgcaaagaaaatcgaagaaaaattcaaacgtgcgcagctgaccctggacaagttcttcaagaaacctaagcttgcggccgcactcgagatcaaacgggctagccagccagaactcgccccggaagaccccgaggatgtcgagcaccaccaccaccaccactga

SEQ ID NO: 80 - DNA, 780 bp, synthetic construct
  misc_feature 1..780: note = SYNTHETIC SEQUENCE
  source 1..780: mol_type = other DNA
atgaaagttgcaggtatagatgaagctggtcgtggtccagctattggtccgctggttattgttgctgctgttgtggatgaaaacagtctccccaagctggaagagctgaaagttagagactccaaaaagctgaccccggcgcaacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgctgatgccgctgatgttgatgaagaacgtttcgctagagagcttggcgagcgtctgaacttcgaagcgaaagttgtagccgaacataaagcggatgcgaagtatgagatcgtatccgcagcctctatcctggcaaaagttatccgtgaccgcgagatcgaaaagctgaaagccgaatacggtgattttggttccggttacccgtctgatccgcgtactagggcattcctggaaaattactatagagaacacggcgaattcccgccgatcgtgcgtaaaggttggaagactctaaagaaaatcgcagaaaaagtcgaaagtgagaagaaggctgaggaacggcaggcgaccctggacaggtacttcaggaaggttaagcttgcggccgcactcgagatcaaacgggctagccagccagaactcgccccggaagaccccgaggatgtcgagcaccaccaccaccaccactga

SEQ ID NO: 81 - DNA, 762 bp, synthetic construct
  misc_feature 1..762: note = SYNTHETIC SEQUENCE
  source 1..762: mol_type = other DNA
atgaaagttgcaggtgcagatgaagctggtcgtggttctgttattggtccgctggttattgttgctgctgttgtggatgaaaacagtctccccaagctggaagagctgaaagttagagactccaaaaagctgaccccgaagcgacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgctgatgccgctgatgttgatgaagaacgtttcgctagagagcttggcgagcgtctgaacttcgaagcggaagttgtagccaaacataaagcggatgacatctttccggtcgtatccgcagcctctatcctggcaaaagttatccgtgaccgcgagatcgaaaagctgaaagccgaatacggtgattttggttccggttacccgtctgatccgcgtactaagaaatggctggaagaatggtatagcaaacacggcaatttcccgccgatcgtgcgtcgtacttgggatactgcaaagaaaatcgaagaaaaattcaaacgtgcgcagctgaccctggacaagttcttcaagaaacctaagcttgcggccgcactcgagatcaaacgggctagccagccagaactcgccccggaagaccccgaggatgtcgagcaccaccaccaccaccactga

SEQ ID NO: 82 - DNA, 762 bp, synthetic construct
  misc_feature 1..762: note = SYNTHETIC SEQUENCE
  source 1..762: mol_type = other DNA
atgaaagttgcaggtgcagatgaagctggtcgtggtccagttattggtccgctggttattgttgctgctgttgtggatgaaaacagtctccccaagctggaagagctgaaagttagagactccaaaaagctgaccccgaagcgacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgtagatgccgctgatgttgatgaagaacgtttcgctagagagcttggcgagcgtctgaacttcgaagcggaagttgtagccaaacataaagcggatgacatctttccggtcgtatccgcagcctctatcctggcaaaagttatccgtgaccgcgagatcgaaaagctgaaagccgaatacggtgattttggttccggttacccgtctgatccgcgtactaagaaatggctggaagaatggtatagcaaacacggcaatttcccgccgatcgtgcgtcgtacttgggatactgcaaagaaaatcgaagaaaaattcaaacgtgcgcagctgaccctggacaagttcttcaagaaacctaagcttgcggccgcactcgagatcaaacgggctagccagccagaactcgccccggaagaccccgaggatgtcgagcaccaccaccaccaccactga

SEQ ID NO: 83 - DNA, 762 bp, synthetic construct
  misc_feature 1..762: note = SYNTHETIC SEQUENCE
  source 1..762: mol_type = other DNA
atgaaagttgcaggtgcagatgaagctggtcgtggttctgttattggtccgctggttattgttgctgctgttgtggatgaaaacagtctccccaagctggaagagctgaaagttagagactccaaaaagctgaccccgaagcgacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgtagatgccgctgatgttgatgaagaacgtttcgctagagagcttggcgagcgtctgaacttcgaagcggaagttgtagccaaacataaagcggatgacatctttccggtcgtatccgcagcctctatcctggcaaaagttatccgtgaccgcgagatcgaaaagctgaaagccgaatacggtgattttggttccggttacccgtctgatccgcgtactaagaaatggctggaagaatggtatagcaaacacggcaatttcccgccgatcgtgcgtcgtacttgggatactgcaaagaaaatcgaagaaaaattcaaacgtgcgcagctgaccctggacaagttcttcaagaaacctaagcttgcggccgcactcgagatcaaacgggctagccagccagaactcgccccggaagaccccgaggatgtcgagcaccaccaccaccaccactga

SEQ ID NO: 84 - DNA, 780 bp, synthetic construct
  misc_feature 1..780: note = SYNTHETIC SEQUENCE
  source 1..780: mol_type = other DNA
atgaaagttgcaggtatagatgaagctggtcgtggttctgctattggtccgctggttattgttgctgctgttgtggatgaaaacagtctccccaagctggaagagctgaaagttagagactccaaaaagctgaccccggcgcaacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgctgatgccgctgatgttgatgaagaacgtttcgctagagagcttggcgagcgtctgaacttcgaagcgaaagttgtagccgaacataaagcggatgcgaagtatgagatcgtatccgcagcctctatcctggcaaaagttatccgtgaccgcgagatcgaaaagctgaaagccgaatacggtgattttggttccggttacccgtctgatccgcgtactagggcattcctggaaaattactatagagaacacggcgaattcccgccgatcgtgcgtaaaggttggaagactctaaagaaaatcgcagaaaaagtcgaaagtgagaagaaggctgaggaacggcaggcgaccctggacaggtacttcaggaaggttaagcttgcggccgcactcgagatcaaacgggctagccagccagaactcgccccggaagaccccgaggatgtcgagcaccaccaccaccaccactga

SEQ ID NO: 85 - DNA, 780 bp, synthetic construct
  misc_feature 1..780: note = SYNTHETIC SEQUENCE
  source 1..780: mol_type = other DNA
atgaaagttgcaggtatagatgaagctggtcgtggtccagctattggtccgctggttattgttgctgctgttgtggatgaaaacagtctccccaagctggaagagctgaaagttagagactccaaaaagctgaccccggcgcgccgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgctgatgccgctgatgttgatgaagaacgtttcgctagagagcttggcgagcgtctgaacttcgaagcgaaagttgtagccgaacataaagcggatgcgaagtatgagatcgtatccgcagcctctatcctggcaaaagttatccgtgaccgcgagatcgaaaagctgaaagccgaatacggtgattttggttccggttacccgtctgatccgcgtactagggcattcctggaaaattactatagagaacacggcgaattcccgccgatcgtgcgtaaaggttggaagactctaaagaaaatcgcagaaaaagtcgaaagtgagaagaaggctgaggaacggcaggcgaccctggacaggtacttcaggaaggttaagcttgcggccgcactcgagatcaaacgggctagccagccagaactcgccccggaagaccccgaggatgtcgagcaccaccaccaccaccactga

SEQ ID NO: 86 - DNA, 780 bp, synthetic construct
  misc_feature 1..780: note = SYNTHETIC SEQUENCE
  source 1..780: mol_type = other DNA
atgaaagttgcaggtatagatgaagctggtcgtggtccagctattggtccgctggttattgttgctgctgttgtggatgaaaacagtctccccaagctggaagagctgaaagttagagactccaaaaagctgaccccggcgcaacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgctgatgccgtagatgttgatgaagaacgtttcgctagagagcttggcgagcgtctgaacttcgaagcgaaagttgtagccgaacataaagcggatgcgaagtatgagatcgtatccgcagcctctatcctggcaaaagttatccgtgaccgcgagatcgaaaagctgaaagccgaatacggtgattttggttccggttacccgtctgatccgcgtactagggcattcctggaaaattactatagagaacacggcgaattcccgccgatcgtgcgtaaaggttggaagactctaaagaaaatcgcagaaaaagtcgaaagtgagaagaaggctgaggaacggcaggcgaccctggacaggtacttcaggaaggttaagcttgcggccgcactcgagatcaaacgggctagccagccagaactcgccccggaagaccccgaggatgtcgagcaccaccaccaccaccactga

SEQ ID NO: 87 - DNA, 780 bp, synthetic construct
  misc_feature 1..780: note = SYNTHETIC SEQUENCE
  source 1..780: mol_type = other DNA
atgaaagttgcaggtatagatgaagctggtcgtggttctgctattggtccgctggttattgttgctgctgttgtggatgaaaacagtctccccaagctggaagagctgaaagttagagactccaaaaagctgaccccggcgcaacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgctgatgccgtagatgttgatgaagaacgtttcgctagagagcttggcgagcgtctgaacttcgaagcgaaagttgtagccgaacataaagcggatgcgaagtatgagatcgtatccgcagcctctatcctggcaaaagttatccgtgaccgcgagatcgaaaagctgaaagccgaatacggtgattttggttccggttacccgtctgatccgcgtactagggcattcctggaaaattactatagagaacacggcgaattcccgccgatcgtgcgtaaaggttggaagactctaaagaaaatcgcagaaaaagtcgaaagtgagaagaaggctgaggaacggcaggcgaccctggacaggtacttcaggaaggttaagcttgcggccgcactcgagatcaaacgggctagccagccagaactcgccccggaagaccccgaggatgtcgagcaccaccaccaccaccactga

SEQ ID NO: 88 - protein, 222 aa, synthetic construct
  REGION 1..222: note = SYNTHETIC SEQUENCE
MKVAGADEAGRGSVIGPLVIVAAVVDENSLPKLEELKVRDSKKLTPKRREKLFDEIVKVLDDYSVVIVSPQDIDGRKGSMNELEVENFVKALNSLKVKPDVIYADAADVDEERFARELGERLNFEAEVVAKHKADDIFPVVSAASILAKVIRDREIEKLKAEYGDFGSGYPSDPRTKKWLEEWYSKHGNFPPIVRRTWDTAKKIEEKFKRAQLTLDKFFKKP

SEQ ID NO: 89 - protein, 222 aa, synthetic construct
  REGION 1..222: note = SYNTHETIC SEQUENCE
MKVAGADEAGRGPVIGPLVIVAAVVDENSLPKLEELKVRDSKKLTPKRREKLFDEIVKVLDDYSVVIVSPQDIDGRKGSMNELEVENFVKALNSLKVKPDVIYADAVDVDEERFARELGERLNFEAEVVAKHKADDIFPVVSAASILAKVIRDREIEKLKAEYGDFGSGYPSDPRTKKWLEEWYSKHGNFPPIVRRTWDTAKKIEEKFKRAQLTLDKFFKKP

SEQ ID NO: 90 - protein, 222 aa, synthetic construct
  REGION 1..222: note = SYNTHETIC SEQUENCE
MKVAGADEAGRGSVIGPLVIVAAVVDENSLPKLEELKVRDSKKLTPKRREKLFDEIVKVLDDYSVVIVSPQDIDGRKGSMNELEVENFVKALNSLKVKPDVIYADAVDVDEERFARELGERLNFEAEVVAKHKADDIFPVVSAASILAKVIRDREIEKLKAEYGDFGSGYPSDPRTKKWLEEWYSKHGNFPPIVRRTWDTAKKIEEKFKRAQLTLDKFFKKP

SEQ ID NO: 91 - protein, 228 aa, synthetic construct
  REGION 1..228: note = SYNTHETIC SEQUENCE
MKVAGIDEAGRGSAIGPLVIVAAVVDENSLPKLEELKVRDSKKLTPAQREKLFDEIVKVLDDYSVVIVSPQDIDGRKGSMNELEVENFVKALNSLKVKPDVIYADAADVDEERFARELGERLNFEAKVVAEHKADAKYEIVSAASILAKVIRDREIEKLKAEYGDFGSGYPSDPRTRAFLENYYREHGEFPPIVRKGWKTLKKIAEKVESEKKAEERQATLDRYFRKV

SEQ ID NO: 92 - protein, 228 aa, synthetic construct
  REGION 1..228: note = SYNTHETIC SEQUENCE
MKVAGIDEAGRGPAIGPLVIVAAVVDENSLPKLEELKVRDSKKLTPARREKLFDEIVKVLDDYSVVIVSPQDIDGRKGSMNELEVENFVKALNSLKVKPDVIYADAADVDEERFARELGERLNFEAKVVAEHKADAKYEIVSAASILAKVIRDREIEKLKAEYGDFGSGYPSDPRTRAFLENYYREHGEFPPIVRKGWKTLKKIAEKVESEKKAEERQATLDRYFRKV

SEQ ID NO: 93 - protein, 228 aa, synthetic construct
  REGION 1..228: note = SYNTHETIC SEQUENCE
MKVAGIDEAGRGPAIGPLVIVAAVVDENSLPKLEELKVRDSKKLTPAQREKLFDEIVKVLDDYSVVIVSPQDIDGRKGSMNELEVENFVKALNSLKVKPDVIYADAVDVDEERFARELGERLNFEAKVVAEHKADAKYEIVSAASILAKVIRDREIEKLKAEYGDFGSGYPSDPRTRAFLENYYREHGEFPPIVRKGWKTLKKIAEKVESEKKAEERQATLDRYFRKV

SEQ ID NO: 94 - protein, 228 aa, synthetic construct
  REGION 1..228: note = SYNTHETIC SEQUENCE
MKVAGIDEAGRGSAIGPLVIVAAVVDENSLPKLEELKVRDSKKLTPAQREKLFDEIVKVLDDYSVVIVSPQDIDGRKGSMNELEVENFVKALNSLKVKPDVIYADAVDVDEERFARELGERLNFEAKVVAEHKADAKYEIVSAASILAKVIRDREIEKLKAEYGDFGSGYPSDPRTRAFLENYYREHGEFPPIVRKGWKTLKKIAEKVESEKKAEERQATLDRYFRKV

SEQ ID NO: 95 - DNA, 669 bp, synthetic construct
  misc_feature 1..669: note = SYNTHETIC SEQUENCE
  source 1..669: mol_type = other DNA
atgaaagttgcaggtgcagatgaagctggtcgtggttctgttattggtccgctggttattgttgctgctgttgtggatgaaaacagtctccccaagctggaagagctgaaagttagagactccaaaaagctgaccccgaagcgacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgctgatgccgctgatgttgatgaagaacgtttcgctagagagcttggcgagcgtctgaacttcgaagcggaagttgtagccaaacataaagcggatgacatctttccggtcgtatccgcagcctctatcctggcaaaagttatccgtgaccgcgagatcgaaaagctgaaagccgaatacggtgattttggttccggttacccgtctgatccgcgtactaagaaatggctggaagaatggtatagcaaacacggcaatttcccgccgatcgtgcgtcgtacttgggatactgcaaagaaaatcgaagaaaaattcaaacgtgcgcagctgaccctggacaagttcttcaagaaaccttga

SEQ ID NO: 96 - DNA, 669 bp, synthetic construct
  misc_feature 1..669: note = SYNTHETIC SEQUENCE
  source 1..669: mol_type = other DNA
atgaaagttgcaggtgcagatgaagctggtcgtggtccagttattggtccgctggttattgttgctgctgttgtggatgaaaacagtctccccaagctggaagagctgaaagttagagactccaaaaagctgaccccgaagcgacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgtagatgccgctgatgttgatgaagaacgtttcgctagagagcttggcgagcgtctgaacttcgaagcggaagttgtagccaaacataaagcggatgacatctttccggtcgtatccgcagcctctatcctggcaaaagttatccgtgaccgcgagatcgaaaagctgaaagccgaatacggtgattttggttccggttacccgtctgatccgcgtactaagaaatggctggaagaatggtatagcaaacacggcaatttcccgccgatcgtgcgtcgtacttgggatactgcaaagaaaatcgaagaaaaattcaaacgtgcgcagctgaccctggacaagttcttcaagaaaccttga

SEQ ID NO: 97 - DNA, 669 bp, synthetic construct
  misc_feature 1..669: note = SYNTHETIC SEQUENCE
  source 1..669: mol_type = other DNA
atgaaagttgcaggtgcagatgaagctggtcgtggttctgttattggtccgctggttattgttgctgctgttgtggatgaaaacagtctccccaagctggaagagctgaaagttagagactccaaaaagctgaccccgaagcgacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgtagatgccgctgatgttgatgaagaacgtttcgctagagagcttggcgagcgtctgaacttcgaagcggaagttgtagccaaacataaagcggatgacatctttccggtcgtatccgcagcctctatcctggcaaaagttatccgtgaccgcgagatcgaaaagctgaaagccgaatacggtgattttggttccggttacccgtctgatccgcgtactaagaaatggctggaagaatggtatagcaaacacggcaatttcccgccgatcgtgcgtcgtacttgggatactgcaaagaaaatcgaagaaaaattcaaacgtgcgcagctgaccctggacaagttcttcaagaaaccttga

SEQ ID NO: 98 - DNA, 687 bp, synthetic construct
  misc_feature 1..687: note = SYNTHETIC SEQUENCE
  source 1..687: mol_type = other DNA
atgaaagttgcaggtatagatgaagctggtcgtggttctgctattggtccgctggttattgttgctgctgttgtggatgaaaacagtctccccaagctggaagagctgaaagttagagactccaaaaagctgaccccggcgcaacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgctgatgccgctgatgttgatgaagaacgtttcgctagagagcttggcgagcgtctgaacttcgaagcgaaagttgtagccgaacataaagcggatgcgaagtatgagatcgtatccgcagcctctatcctggcaaaagttatccgtgaccgcgagatcgaaaagctgaaagccgaatacggtgattttggttccggttacccgtctgatccgcgtactagggcattcctggaaaattactatagagaacacggcgaattcccgccgatcgtgcgtaaaggttggaagactctaaagaaaatcgcagaaaaagtcgaaagtgagaagaaggctgaggaacggcaggcgaccctggacaggtacttcaggaaggtttga

SEQ ID NO: 99 - DNA, 687 bp, synthetic construct
  misc_feature 1..687: note = SYNTHETIC SEQUENCE
  source 1..687: mol_type = other DNA
atgaaagttgcaggtatagatgaagctggtcgtggtccagctattggtccgctggttattgttgctgctgttgtggatgaaaacagtctccccaagctggaagagctgaaagttagagactccaaaaagctgaccccggcgcgccgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgctgatgccgctgatgttgatgaagaacgtttcgctagagagcttggcgagcgtctgaacttcgaagcgaaagttgtagccgaacataaagcggatgcgaagtatgagatcgtatccgcagcctctatcctggcaaaagttatccgtgaccgcgagatcgaaaagctgaaagccgaatacggtgattttggttccggttacccgtctgatccgcgtactagggcattcctggaaaattactatagagaacacggcgaattcccgccgatcgtgcgtaaaggttggaagactctaaagaaaatcgcagaaaaagtcgaaagtgagaagaaggctgaggaacggcaggcgaccctggacaggtacttcaggaaggtttga

SEQ ID NO: 100 - DNA, 687 bp, synthetic construct
  misc_feature 1..687: note = SYNTHETIC SEQUENCE
  source 1..687: mol_type = other DNA
atgaaagttgcaggtatagatgaagctggtcgtggtccagctattggtccgctggttattgttgctgctgttgtggatgaaaacagtctccccaagctggaagagctgaaagttagagactccaaaaagctgaccccggcgcaacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgctgatgccgtagatgttgatgaagaacgtttcgctagagagcttggcgagcgtctgaacttcgaagcgaaagttgtagccgaacataaagcggatgcgaagtatgagatcgtatccgcagcctctatcctggcaaaagttatccgtgaccgcgagatcgaaaagctgaaagccgaatacggtgattttggttccggttacccgtctgatccgcgtactagggcattcctggaaaattactatagagaacacggcgaattcccgccgatcgtgcgtaaaggttggaagactctaaagaaaatcgcagaaaaagtcgaaagtgagaagaaggctgaggaacggcaggcgaccctggacaggtacttcaggaaggtttga

SEQ ID NO: 101 - DNA, 687 bp, synthetic construct
  misc_feature 1..687: note = SYNTHETIC SEQUENCE
  source 1..687: mol_type = other DNA
atgaaagttgcaggtatagatgaagctggtcgtggttctgctattggtccgctggttattgttgctgctgttgtggatgaaaacagtctccccaagctggaagagctgaaagttagagactccaaaaagctgaccccggcgcaacgtgaaaaactgttcgatgaaatcgtaaaagtactggatgattactctgtggtcattgtgtccccgcaggacattgacggtcgtaagggcagcatgaacgaactggaggtagaaaacttcgttaaagccctgaatagcctgaaagttaagccggatgttatttacgctgatgccgtagatgttgatgaagaacgtttcgctagagagcttggcgagcgtctgaacttcgaagcgaaagttgtagccgaacataaagcggatgcgaagtatgagatcgtatccgcagcctctatcctggcaaaagttatccgtgaccgcgagatcgaaaagctgaaagccgaatacggtgattttggttccggttacccgtctgatccgcgtactagggcattcctggaaaattactatagagaacacggcgaattcccgccgatcgtgcgtaaaggttggaagactctaaagaaaatcgcagaaaaagtcgaaagtgagaagaaggctgaggaacggcaggcgaccctggacaggtacttcaggaaggtttga